(19) United States
(12) Patent Application Publication
(10) Pub. No.: US 2024/0315646 A1
(43) Pub. Date: Sep. 26, 2024

(54) WIRELESS HEART TELEMETRY ELECTRODES AND PATIENT MONITORING SYSTEM

(71) Applicant: JMad Creations, LLC, Bethlehem, PA (US)

(72) Inventor: Jonathan M. MADERIC, Bethlehem, PA (US)

(73) Assignee: JMad Creations, LLC, Bethlehem, PA (US)

(21) Appl. No.: 18/407,432

(22) Filed: Jan. 8, 2024

Related U.S. Application Data
(63) Continuation-in-part of application No. 18/087,519, filed on Dec. 22, 2022, now Pat. No. 11,864,923.
Application No. 18/087,519 is a division of application No. 17/883,068, filed on Aug. 8, 2022, now Pat. No. 11,864,921.
(60) Provisional application No. 63/230,815, filed on Aug. 9, 2021.

(51) Int. Cl. A61B 5/00; A61B 5/0205; A61B 5/024; A61B 5/1455

(52) U.S. Cl. CPC A61B 5/7203; A61B 5/002; A61B 5/0205; A61B 5/14552; A61B 5/681; A61B 5/7225; A61B 5/742; A61B 5/02433

(57) ABSTRACT

The wireless heart telemetry device and accompanied patient monitoring system provides continuous patient monitoring in a comfortable and streamlined design for healthcare settings by providing electrocardiogram (ECG) physiological data in a wireless skin patch device. The wireless heart telemetry device integrates into a full patient monitoring system to provide additional vital physiological patient data in wireless system. This additional physiological data includes pleth, blood oxygen saturation (SPO₂), blood pressure, heart rate, respiration rate, temperature, and glucose. The pulse oximeter portion of the system includes mechanical designs such as a finger clip, ring, and bracelet design for enhanced usage, accuracy, and comfort. Another example embodiment of the pulse oximeter portion of the system includes fall detection, bed alarm, and location monitoring services. An additional example embodiment includes a melanin bias reducing pulse oximeter.

### CROSS-REFERENCE TO RELATED APPLICATIONS

This application is a continuation-in-part of U.S. application Ser. No. 17/883,068 filed on Aug. 8, 2022, which claims benefit of priority from U.S. Provisional Application No. 63/230,815 filed on Aug. 9, 2021, the entire contents of each of which are incorporated by reference.

### TECHNICAL FIELD

The technology relates to pulse oximeters, heart rate monitors, electrocardiogram (ECG) systems, and other patient monitoring devices. More specifically, the technology relates to hardware, software, integrated circuits and components for wireless heart telemetry such as wireless heart telemetry electrodes, as well as a user interface and patient monitoring system. These patient monitoring systems include pulse oximetry, which includes pulse oximeters that reduce the melanin bias found in current pulse oximeters, as well as other devices to collect additional physiological data.

### BACKGROUND

There are currently heart telemetry electrodes on the market that allow for ECG (electrocardiogram) data to be presented to the user, via on-board display, wireless technology, smart devices, or patient monitoring system displays. These electrodes connect to a patient using a conductive pad attached to an adhesive sticker which is placed on the skin. The combination of the electrical signals detected between the different pad locations in the array or network of telemetry pads provides the ECG processor with the required physiological information to determine a wave form representing the electrical activity of the heart.

The abbreviation ECG and EKG are used interchangeably in the medical community when discussing electrocardiograms. The abbreviation EKG comes from the original German spelling for electrocardiogram and is still at times used today in the US and other parts of the world. Current heart telemetry electrodes and/or pads work well. However, they require multiple cables to be connected to each individual pad on the patient. These cables are often connected to a wall mounted or bedside monitoring system, which poses a problem for patient ambulation, patient comfort, and patient safety. These systems often require disconnecting the patient from critical monitoring systems when the patient needs to use the restroom, to shower, to be moved to a procedure, or to change clothing. These systems can also include additional wired connections for pulse oximeters, temperature probes, glucose probes, and blood pressure (BP) cuffs for reading additional physiological information from the patient.

Other systems include a wireless local receiver, as further discussed in this document. In these systems, the heart telemetry electrodes connect to the local receiver, which is often placed in a patient's hospital gown pocket or clipped to the patient's belt or waist. In some cases, the transmitter is a unit that is removable from the wall unit to be brought with the patient when being moved to procedures. The receiver wirelessly transmits the information to the hospital monitoring system. These systems however, still require individual cables to be connected to each individual pad/electrode. These cables still pose a risk to the patient, often get tangled in the patient's gown, are uncomfortable, and must still be disconnected when bathing and at times when using the restroom or changing clothing. Ambulation of a patient in emergency department and ambulatory surgical unit settings in which monitors are often removed from the patient during ambulation poses a significant risk, as ambulation is often a critical time to monitor the patient. Monitoring the patient is also critically important when they use the restroom as the patient is in a situation where nurse “visual” monitoring is often not performed and the rapid changes in posture can pose a significant cardiovascular risk. However, due to the wired monitoring systems, a patient is typically disconnected from the wired monitoring system prior to using the restroom, as most emergency department and ambulatory surgical unit monitoring systems are tethered to the wall at the bedside. Even in systems that provide cumbersome wireless ECG monitoring, the patients' blood pressure and oxygen saturation, via pulse oximetry, are often not included measurements in these systems and therefore do not provide an accurate visualization of the patient's overall vitals. These systems often still require additional cables for additional wired connections for pulse oximeters, temperature probes, glucose probes, and blood pressure (BP) cuffs for reading additional physiological information from the patient, which would need to be disconnected during ambulation, thus losing additional critical vitals monitoring data.

There are currently pulse oximeters on the market that allow for heart rate, pulse oximetry and plethysmograph (pleth) graph data to be presented to the user, via on-board display, wireless technology, smart device, or patient monitoring system displays. These pulse oximeters operate as stand-alone devices, networked, and/or wireless devices. Displays used for these pulse oximeters are either light emitting diode (LED) arrays/matrices, liquid crystal displays (LCDs), or organic light emitting diodes (OLEDs). These pulse oximeters use two different wavelengths of LEDs to operate. One LED is a red LED normally in the 640 nm range, and the other LED is an IR LED normally in the 940 nm range. Some pulse oximeters use red and IR LED combinations in other, but similar wavelengths. These pulse oximeters use the data from both LEDs, via a photodiode, in order to calculate the blood oxygen saturation level in a patient. The heart rate is calculated by using the data from only one LED, typically the IR LED. It is possible to get a blood oxygen saturation reading, via these two LEDs, because these LEDs' wavelengths are on opposite sides of the isosbestic point of the absorption rates for oxyhemoglobin and deoxyhemoglobin for these wavelengths. The isosbestic point is the specific wavelength at which the total absorption of a material does not change during a physical change in the sample. In the case of hemoglobin this is the point where the absorption rates of oxyhemoglobin and deoxyhemoglobin are the same. Oxyhemoglobin is the oxygenated hemoglobin in the blood, and deoxyhemoglobin is the deoxygenated hemoglobin in the blood.

The problem experienced by many of these pulse oximeters is that the red light is absorbed and scattered by melanin that resides in the skin, and provides falsely high blood oxygen saturation readings on patients with skin including higher concentrations of melanin. Blood oxygen saturation (SpO₂) is an important medical measurement used for determining the percentage of oxygen in the blood and is routinely used for guiding decisions on oxygen therapy requirements for patients. Blood oxygen saturation is also an important measurement for monitoring during anesthesia administration and assists in determining cardiac and respiratory function. Falsely high blood oxygen saturation readings in a patient can delay critical and often lifesaving oxygen therapy treatment and can provide inaccurate data when administrating anesthesia, which can lead to a high disparity of care. Melanin is a dark brown or black pigment in skin that is also responsible for tanning when exposed to sunlight. The darker the skin, the larger the concentration of melanin, and therefore, a higher amount of melanosomes, where melanosomes are the lipid bilayer bound organelle that produces melanin. Absorption occurs when light or photons are completely blocked from passing through the materials in question due to the materials “taking in” the light or photons. Scattering occurs due to the bouncing of light or photons at incident angles due to the contacting of the material or object in question. The melanin and melanosomes both contribute to the light disruption, via absorption and scattering. It has also been found that the lipid bilayers scatter light depending on the concentration of the bilayers and the intensity and type of the incoming light.

Overall scattering occurs at a higher rate at lower wavelengths, although scattering does occur across the light spectrum. Absorption, however mainly occurs in the melanin found in the epidermis mostly located in the basal layer where high concentrations of melanosomes are found. The two types of melanin that pose the largest absorption problem are pheomelanin and eumelanin, which have different effects on skin color and therefore cause different wavelengths to be absorbed at different rates. Pheomelanin portrays a red/yellow color, while eumelanin portrays a brown/red color. Therefore, in current pulse oximeters that use red light, although pheomelanin causes absorption problems, eumelanin causes a higher rate of absorption and therefore a higher rate of falsely high pulse oximetry readings. Eumelanin concentration is directly proportional to the shade of skin color and is mostly responsible for the overall darkness of the skin, where pheomelanin has a more constant trend across the shades of skin color and is primarily responsible for the yellow/red tint in skin color. Therefore, the shade of skin color is directly proportional to the concentration of eumelanin in the skin, and is the type of melanin that is responsible for the greatest bias causing falsely high blood oxygen saturation readings.

The user interface for many pulse oximeters is a single-color output that can be difficult for non-medical patients, laypersons, or personnel to interpret blood oxygen saturation levels. These screens often have a single low battery indicator that does not accurately display the remaining battery life of the meter. Also, many meters often use disposable batteries, rather than rechargeable batteries.

Many pulse oximeters currently on the market use a design that clips onto fingers or toes and can be cumbersome to wear for extended periods of time. On patients with poor circulation, the meters are currently taped or clipped onto the ear, which is not an ideal placement and the cables can become a hazard to the patient. For infants, the meters are often taped or wrapped around a leg or wrist. For toddlers, the meters are often taped or wrapped around a finger or toe because the meters currently on the market do not accommodate smaller appendages. These meters often fall off and can also reduce the accuracy of the blood oxygen saturation and heart rate readings and results. In many cases, the meter designs previously mentioned, are extremely sensitive to patient movement, which can result in inaccurate readings as well. These designs at best are water resistant and not waterproof, posing a problem for patients that must wear these meters for extended periods of time.

Some newer pulse oximeter designs utilize non-flexible rings which cause these meters to be ineffective when used on individuals with smaller fingers and/or toddlers/infants. Additionally, non-flexible rings can pose an additional health risk in medical situations where swelling of the appendages can occur, therefore reducing overall circulation in the appendage. Also, some designs utilize IR LEDs to measure pulse oximetry readings using the patients' foreheads and lack the comfort and other features that the invention discussed in this document provides. Further, current pulse oximeters often provide false readings when used on individuals wearing nail polish.

### SUMMARY

The invention solves many of the issues mentioned in the background section, via different means depending on the embodiment. One example embodiment of the invention uses the finger clip design, in either a standalone or connected patient monitoring system, similar to the physical designs currently on the market, however, utilizing the melanin bias reducing blood oxygen saturation measurement method herein. Another example embodiment uses a flexible ring design, and another example embodiment uses a flexible bracelet design to alleviate many of the placement concerns noted with the current pulse oximeters on the market. An example embodiment of the ring design uses material and casing to ensure waterproof operation. The ring and bracelet embodiments are sized, depending on application, to work for both infant monitoring on wrists or legs, as well as adult and pediatric monitoring on fingers. The ring and bracelet embodiments' sizing also allows for ease of use for patients who may be amputees or patients who are unable to wear traditional pulse oximeters for reasons such as for example, but not limited to, anatomy, age, mental disability, sensitivity issues, and/or attention deficit disorder (ADD/ADHD). Example embodiments of the ring and bracelet embodiments include, but are not limited to, an embodiment with a built-in screen, an embodiment with a smart device display, an embodiment with a wrist mounted screen, and/or an embodiment connected to a patient monitoring system. Further, other example embodiments of the bracelet are designed in such a way that they incorporate a wearable flexible band.

One example embodiment of the invention reduces the melanin issue previously discussed by using 2 IR LEDs, about 768 nm and about 940 nm, along with an analog photodiode and accompanying analog circuits and software. Another example embodiment of the invention uses a digital photodiode, along with accompanying software to solve this problem. Yet another example embodiment of the invention uses a phototransistor, along with accompanying hardware and software to solve this problem. In order to determine the best LEDs and detectors (analog/digital) to use, one example embodiment of a test bench system is used. This example embodiment of the test bench system uses serial dilutions of synthetic melanin used to dye pig skin to represent different concentrations of melanin in human skin. In this example embodiment, the test bench measures the intensity of light received, after passing through different concentrations of melanin dyed pig skin, through a variety of different tests.

One example embodiment of the invention uses a user interface that displays pleth graph, blood oxygen saturation level, heart rate, and battery meter. One example embodiment of the screen layout changes color when the pulse oximeter detects good (95-100%), moderate (90-95%), or critical (<90%) blood oxygen saturation level readings to increase ease of use and help with blood oxygen saturation level interpretations especially when used by a layperson. Another example embodiment of the screen layout uses a segmented battery meter that changes color to indicate a good battery, almost discharged battery, and battery that needs to be recharged after the next few uses.

One example embodiment of the invention uses disposable batteries as its power source. Another example embodiment uses rechargeable batteries. Further, another example embodiment uses a wall adaptor, such as, for example, in hospital patient monitoring systems. Further, another example embodiment uses a wall adaptor with a built-in battery backup, such as, for example, in other hospital patient monitoring systems. These example embodiments use many different types and styles of batteries depending on the embodiment and its space, weight, and power consumption requirements.

One example embodiment uses wireless technology for the pulse oximeter to communicate with the user interface. This wireless interface takes on many different embodiments. An example embodiment is a system that wirelessly communicates directly with a patient monitoring system. Yet another example embodiment communicates with a belt or wrist-pack that includes a small user interface and boosts the wireless signal to be transmitted to the hospital patient monitoring system. Further, another example embodiment communicates with a patient monitoring system via wired and wireless networked communications. These wireless and wired embodiments are compatible with all embodiments of the pulse oximeter design, in accordance with the invention.

Another example embodiment of the design includes heart telemetry monitoring built into the pulse oximetry system therefore providing a 5^{th} vital sign as a blood oxygen saturation level to the patient monitoring system. In medicine there are four well known vital signs, which include body temperature, pulse rate, respiration rate (or rate of breathing), and blood pressure. Recently, medicine is considering pulse oximeter's blood oxygen saturation reading (SpO₂) to be considered a 5^{th} vital sign due to the important physiological information it is reporting. SpO₂ tells the percentage of blood that is saturated with oxygen, which can be a representation of lung function and the amount of oxygen reaching the tissues and organs in the body. Another example embodiment of the design includes a heart telemetry monitoring system which includes the pulse oximetry system or pulse oximeter device previously discussed in this document incorporating the 5^{th} vital sign as a blood oxygen saturation level to the patient monitoring system. An example embodiment of these systems includes skin electrodes that are wired to a belt or chest pack that combines this information with the pulse oximetry system (wired or wireless) to be displayed locally or transmitted (wired or wireless) to the patient monitoring system. Another example embodiment of the heart rate telemetry system includes skin electrodes that use individual wireless transmitters rather than wired electrodes to transmit heart rate data along with the pulse oximetry data to either a local belt pack, a bedside monitor, or directly to the patient monitoring system, thus being able to discard cumbersome cables on the patient. Another example embodiment of the heart rate telemetry system uses a waterproof centralized controller, which can be adhered to the patient's chest or other location near the heart, and combines the information from the skin electrodes, via short cables, with the pulse oximeter reading to be displayed wired or wirelessly locally, or wired or wirelessly via the patient monitoring system. In systems such as these the base requirement for operating the monitoring system is the connection of the wireless heart telemetry pads. In these systems it is additionally recommended to connect a wireless ring embodiment of a pulse oximeter to the system for additional physiological information, such as, but not limited to, blood oxygen saturation, heart rate, and pleth information.

Another example embodiment of the design additionally includes at least one of, but not limited to, a blood pressure cuff, a temperature probe, a continuous glucose monitor, a pulse oximeter, a melanin bias reducing pulse oximeter, and other physiological data collection devices in conjunction with wireless heart telemetry pads connected to a wired or wireless patient monitoring system. These devices used in a wireless system either directly connect to a hospital patient monitoring system or they connect to a local bedside, pocket worn, or clip on receiver. Some example embodiments of the receiver include a built-in screen to locally display the patient physiological data. Other example embodiments of the receiver also include wired or wireless communication technology to transmit the physiological data to a patient monitoring system as further described later in this document.

### BRIEF DESCRIPTION OF THE DRAWINGS

FIG. 1 shows an example embodiment of a general hardware layout of an example embodiment of a test bench system, in accordance with the invention, used for performing LED and detector performance tests to achieve a(n) LED(s) and a detector combination to reduce the melanin bias.

FIG. 2 shows an example embodiment of a test bench system including its UI and peripheral interface devices used for testing, in accordance with the invention.

FIG. 3 shows an example embodiment of a general layout, in accordance with the invention, of one example embodiment of an analog detector for a test bench system.

FIG. 4 shows an example embodiment of a general layout, in accordance with the invention, of an example embodiment of a digital detector for a test bench system.

FIG. 5A-5B shows an example embodiment of two example LEDs and their example mounting and connection methods, in accordance with the invention, for a test bench system.

FIG. 6 shows an overview of a general hardware signal flow of an example embodiment of a test bench system, in accordance with the invention.

FIG. 7 shows an overview of a general usage, via the UI (user interface), in accordance with the invention, of an example embodiment of a test bench system.

FIG. 8 shows a general usage method, in accordance with the invention, of an example embodiment of a test bench system with pig skin, detector, emitter, and a blackout box.

FIG. 9 shows a general software flow, in accordance with the invention, of an example embodiment of a test bench system.

FIG. 10 shows a diagram of an example embodiment of a finger clip design, in accordance with the invention, of a pulse oximeter.

FIG. 11 shows a diagram of an example embodiment of a finger clip design, in accordance with the invention, of a pulse oximeter including an example subject/patient finger placement.

FIG. 12 shows a general layout of an example embodiment of a user interface output for pulse oximetry, in accordance with the invention.

FIGS. 13A-13B show an exploded view of an example embodiment of a finger clip design, in accordance with the invention, of a pulse oximeter.

FIG. 14 shows the assembled view of a top half (upper) of an example embodiment of a finger clip design, in accordance with the invention.

FIG. 15 shows an assembled view of a lower half (bottom) of an example embodiment of a finger clip design, in accordance with the invention.

FIG. 16A-16B shows a top and bottom view of an example layout of an example lower (bottom) PCB (printed circuit board) of a pulse oximeter, in accordance with the invention.

FIG. 17A-17B shows a top and bottom view of an example layout of an example upper (top) PCB (printed circuit board) of a pulse oximeter, in accordance with the invention.

FIG. 18 shows a diagram of an example usage of an example embodiment of a ring design of a pulse oximeter, in accordance with the invention.

FIG. 19 shows a diagram of an example usage of an example embodiment of a ring design of a pulse oximeter, in accordance with the invention, including an example embodiment of a UI (user interface).

FIG. 20 shows a diagram of an example design of a flexible ring embodiment of a pulse oximeter, in accordance with the invention.

FIG. 21 shows a diagram of an example design of a flexible ring embodiment of a pulse oximeter with an example embodiment of an example wrist mounted UI.

FIG. 22 shows an overview of a general hardware signal flow of an example embodiment of a pulse oximeter system, in accordance with the invention.

FIG. 23 shows an overview of a general software signal flow of an example embodiment of a pulse oximeter system, in accordance with the invention.

FIG. 24 shows a diagram of an example embodiment of a bracelet or wearable flexible band design, in accordance with the invention, of a pulse oximeter.

FIG. 25 shows an example fall detection and bed alarm monitor usage, in accordance with the invention, of an example ring embodiment of a pulse oximeter.

FIG. 26 shows an example of a patient location tracking triangulation method, in accordance with the invention, of an example ring embodiment of a pulse oximeter.

FIG. 27 shows an example embodiment of a wireless heart telemetry monitoring system with an example embodiment of wireless patient/subject worn conductive pads, in accordance with the invention.

FIG. 28 shows an example embodiment of a wireless heart telemetry monitoring system with an example embodiment of a patient/subject worn telemetry base pack, in accordance with the invention.

FIG. 29 shows an example embodiment of a wireless patient monitoring system including, for example, heart telemetry monitoring and pulse oximetry, in accordance with the invention.

FIG. 30 shows an example embodiment of a wireless patient monitoring system including, for example heart telemetry monitoring, pulse oximetry, blood pressure (BP), temperature, and glucose monitoring, in accordance with the invention.

FIGS. 31A, 31B, 31C, and 31D show top and side view diagrams of an example embodiment of a wireless heart telemetry pad or electrode, as well as the mating of the disposable and reusable portions of the wireless heart telemetry electrode, in accordance with the invention.

FIG. 32 shows a diagram of an overview of an example general hardware signal flow of an example wireless heart telemetry pad or electrode, in accordance with the invention.

FIG. 33 shows a diagram of an example design of a flexible ring embodiment of a pulse oximeter including the internal components arrangement to provide flexible adjustment, in accordance with the invention.

FIGS. 34A-34B show two exploded view diagrams of the example design of a flexible ring embodiment in accordance with the invention. FIG. 35 shows a diagram of another example design of a flexible ring embodiment of a pulse oximeter including the internal components arrangement to provide flexible adjustment, in accordance with the invention.

FIGS. 36A-36B show two exploded views diagrams of the example design of a flexible ring embodiment of a pulse oximeter in accordance with the invention.

FIGS. 37A-37B show side and bottom view diagrams of an example design of a single chip PPG (Photoplethysmography) sensor including melanin bias reducing technology as a drop in smart device and electronics sensor, in accordance with the invention.

FIG. 38 shows an example embodiment of a wireless heart telemetry monitoring system with an example embodiment of wireless patient/subject worn conductive pads that include under gown cables to link the pads to a central wireless heart telemetry pad, and includes an example ring embodiment for combined wireless pulse oximetry, in accordance with the invention.

FIG. 39 shows an example embodiment of a wireless heart telemetry monitoring system with an example embodiment of wireless patient/subject worn conductive pads that include a central wireless heart telemetry electrode with an example straight two pad clip, and includes an example ring embodiment for combined wireless pulse oximetry, in accordance with the invention.

FIG. 40 shows an example embodiment of a wireless heart telemetry monitoring system with an example embodiment of wireless patient/subject worn conductive pads that include a central wireless heart telemetry electrode with an example right angle two pad clip, and includes an example ring embodiment for combined wireless pulse oximetry, in accordance with the invention.

FIG. 41 shows an example embodiment of a wireless heart telemetry monitoring system with an example embodiment of wireless patient/subject worn conductive pads that include a central wireless heart telemetry electrode with an example right angle two pad clip with an additional under gown wired pad connected to the central wireless heart telemetry electrode, and includes an example ring embodiment for combined wireless pulse oximetry, in accordance with the invention.

FIG. 42 shows a general software flow, in accordance with the invention, of an example wireless heart telemetry device.

### DETAILED DESCRIPTION

Systems, in accordance with the invention, include a pulse oximeter device that includes a method for reducing the melanin bias in skin. This pulse oximeter system includes multiple methods for receiving the telemetry, including heart telemetry, and blood oxygen saturation information, wired or wirelessly, through a user interface, for example a patient monitoring system or standalone UI. There are multiple embodiments of this pulse oximeter system that take on different forms, shapes, and attachment methods. Part of the development of this pulse oximeter system included using a testing system for determining the best optical configuration and design for reducing the melanin bias discussed previously in this document. Also included in this document are patient monitoring systems that include wireless heart telemetry devices, as well as integrated pulse oximeter devices and additional physiological information devices and systems as previously discussed.

### Test Bench

The test bench is a system that was developed for determining the best optical arrangement to reduce the melanin bias that causes light absorption and light scattering to interfere with accurate pulse oximetry readings. The test bench includes hardware, software, and physical components, such as, for example, pig skin and synthetic melanin dye. The example embodiment discussed in this section was developed using perf board, as well as through-hole parts, including SMD components mounted onto through-hole converter PCBs. Other example embodiments of the test bench are constructed using custom PCB's and primarily SMD parts to reduce the size and increase the robustness of the device.

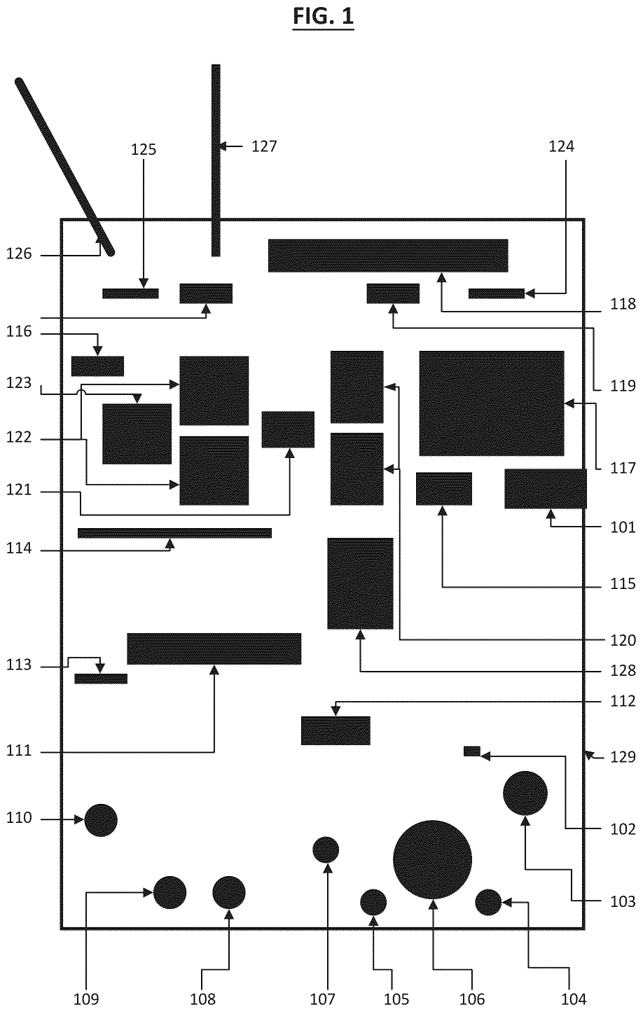

FIG. 1 shows an example embodiment of a general hardware layout of an example embodiment of the test bench system showing general locations of the hardware components. 101 represents the power input barrel adaptor used to connect an AC wall adaptor to power the circuit. Other example embodiments use USB connectors and a USB power source, instead of a barrel jack for 101.

Furthermore, other example embodiments of the test bench use other power sources, including but not limited to, on-board AC/DC converters and/or batteries. 102 represents the header jumper to disable or enable the buzzer output part of the user interface. Other example embodiments of the test bench use digital or analog methods to control the buzzer, including, but not limited to, GPIO controlled switching methods. 103 represents the buzzer part of the user interface that emits sound based on user input and program actions. Other example embodiments of the test bench use other forms of audio and/or visual indicators instead of a buzzer, such as, but not limited to, speakers or audio and/or voice synthesizers. The user input block includes 104-107. 104 represents the tactile switch used for moving right by one digit on display 201. 105 represents the tactile switch used for moving left by one digit on display 201. 106 represents the rotary encoder used to move through digits on display 201 and to detect enter presses. 107 represents the menu select button used to move through different menus on LCD screen 201. The user input block in other example embodiments includes other input methods, such as, but not limited to, multiple rotary encoders, touch screens, keyboards, keypads, and/or computer and smart device interfaces. The LCD control block includes 108-109. 108 represents the display, 201, brightness adjustment potentiometer. 109 represents the display, 201, contrast adjustment potentiometer. In other example embodiments, the LCD control block is replaced with other analog or digital methods of control, including, but not limited to, fixed resistor values, GPIO control, digital potentiometer control, and/or digital to analog converter control (DAC). Other example embodiments in which different types of displays, 201, are used have different control blocks, 108-109, depending on the display technology used. 110 represents the microcontroller, 111, reset button to reset the microcontroller in case of a program halt. Other example embodiments replace the reset button with watch dog timers. 111 represents the MCU (microcontroller unit). In the example embodiment shown, an Atmel ATMEGA 328P is used for the MCU 111. Other example embodiments of the test bench use other types of microcontrollers with similar features and functions. 112 represents the crystal oscillator used as the clock for MCU 111. In the example embodiment of the test bench discussed here, 112 is a 16 MHz clock, in order to allow for proper software performance. 113 represents the programming header used for reprogramming MCU 111. 114 represents the alpha numeric LCD screen header that the screen 201 attaches to. Other example embodiments of the test bench use other methods of attachment depending on the display type used. 115 represents the fusing block used to protect the test bench in the event of a short. 116 represents the power regulation block that produces both the 3.3V and 5V source required to power the test bench. Other example embodiments of the invention use other voltage sources and power supply methods to power the test bench. 117 represents the current monitoring control block, which is connected to current shunt 118 for calibration purposes. 119 represents the calibration controller used to calibrate the current meter 117 during initial boot-up. 120 represents the LED current driver block used in conjunction with 121 and 122 to create a current source for the LEDs used in testing. 121 represents the op-amp, placed in comparator mode, and is part of the current source. 122 represents the DAC section of the current source. Other example embodiments use other methods for the DAC portion of the current source that generates a programmable voltage output including, but not limited to, PWM filtered analog voltages. Further, other example embodiments use other methods to produce a controllable current source for the LEDs, such as, but not limited to, current source ICs, digitally controllable regulators, or digitally controllable current source supplies. 123 represents the level shifter block for the digital detector, 203. Other example embodiments of the test bench use other types of digital detectors that do not require voltage level shifting at block 123. 124 represents the analog detector, 204, connector and 125 represents the digital detector, 203, connector. One example embodiment of these connectors, 124 and 125, is a header style connector. Other example embodiments of connectors 124 and 125, include, but are not limited to, Molex connectors, ZIF connectors, gold fingers, ribbon cable connectors, and/or JST connectors. 126 represents the main LED cable and 127 represents the secondary LED cable. Other example embodiments use other methods for attaching LEDs to the test bench system, including, but not limited to, snap connectors, JST connectors, headers, barrel jacks, Molex connectors, and/or butt connectors. 128 represents the reverse current protection control block of the circuit to protect the testing system from inadvertent inverse polarity power supplies connected via barrel jack, 101. 129 represents the main board that all components previously discussed for the test bench are mounted to. One example embodiment of the main board is perf board with a plastic cover to protect the solder joints. Another example embodiment of the main board is the printed circuit board (PCB) with a plastic cover to protect the traces.

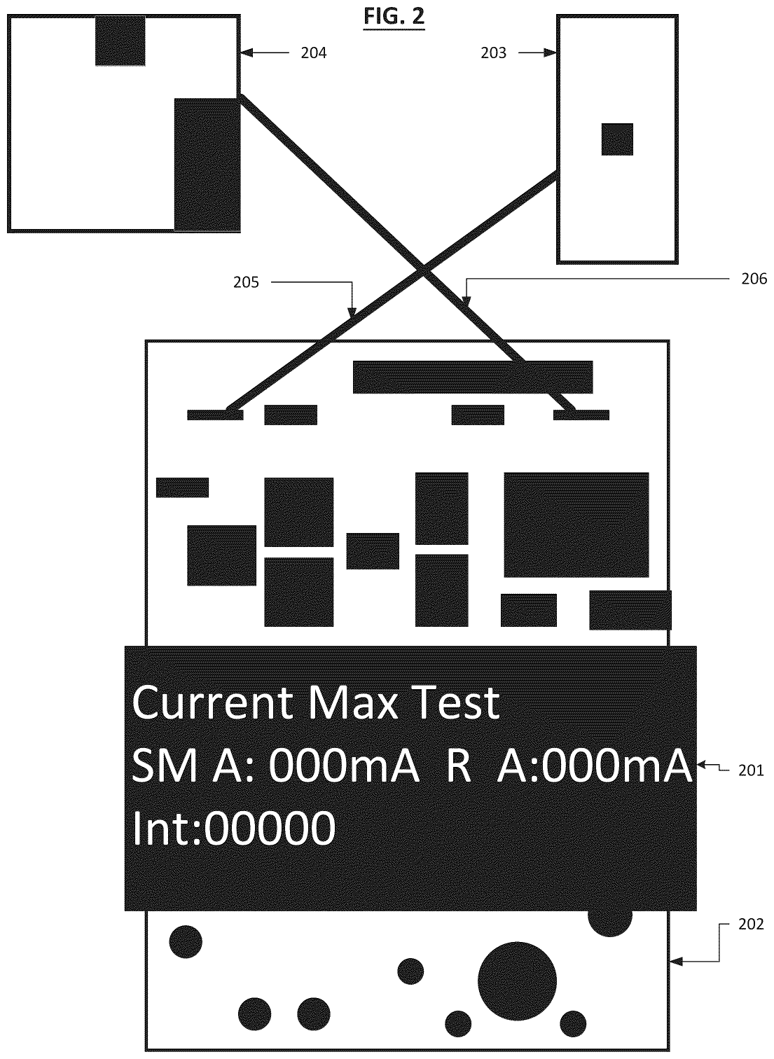

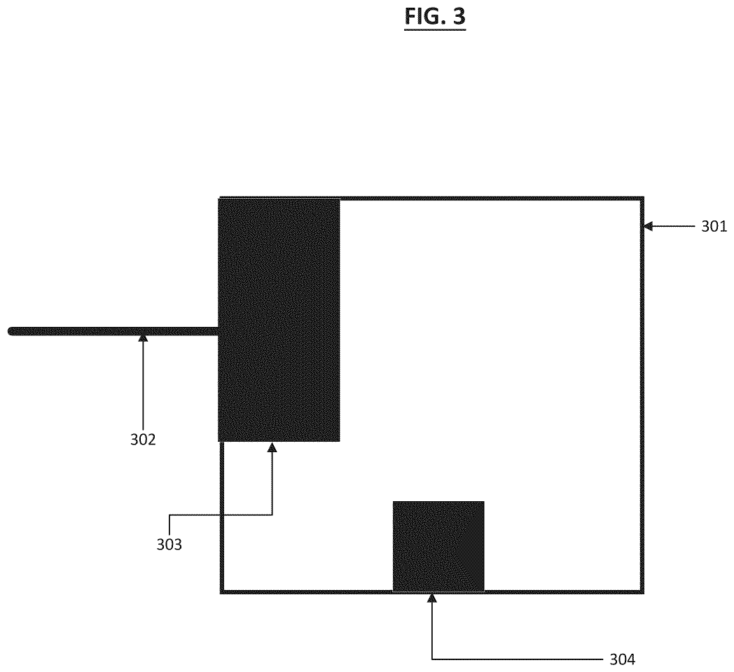

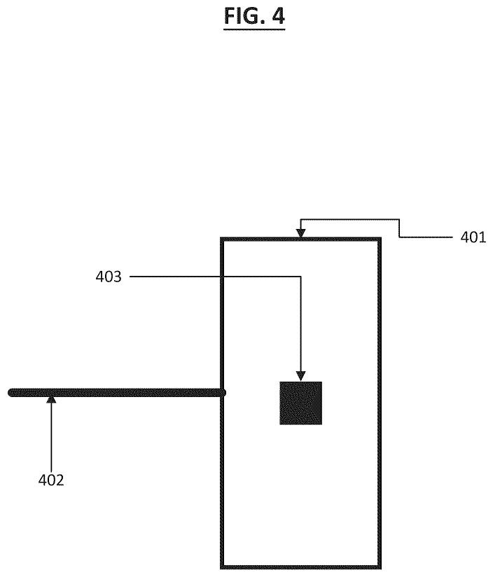

FIG. 2 shows an example embodiment of the test bench system including its user interface and detectors. 201 represents the alpha numeric screen mounted on the test bench. Other example embodiments of the test bench use other display methods for screen 201, such as, but not limited to, LCD, OLED, E-ink, computer display, and/or smart device display. 202 represents the main board as described before (board 129). 203 represents the digital detector described further in FIG. 4. 204 represents the analog detector described further in FIG. 3. 205 and 206 represent the ribbon cables that provide power and data transmission for the digital and analog detectors respectively. Other example embodiments of the test bench system use other connection methods instead of ribbon cables 205 and 206, such as, but not limited to, FPC cables, multi-core cables, and/or twisted pair cables.

FIG. 3 shows an example embodiment of a general layout of the analog detector circuit. 301 represents the main analog detector board. In the example embodiment shown, board 301 is a piece of perf board using through-hole parts. Other example embodiments of the test bench use SMD parts and custom PCBs for board 301. 302 represents the cable discussed as 206 in FIG. 2. 303 represents the high-resolution analog to digital converter (ADC) which converts the voltages from photodiode 304 to data for MCU 111. 304 represents the photodiode with a built-in transimpedance amplifier. A transimpedance amplifier is required to convert the low current signals produced by the photodiode into voltages useable by the ADC, 303. Other example embodiments of the analog detector circuit use separate analog photodiodes and transimpedance amplifiers instead of the all-in-one IC shown in 304.

Other example embodiments of the analog detector circuit use separate analog phototransistors instead of the all-in-one IC shown in 304.

FIG. 4 shows an example embodiment of a general layout of the digital detector circuit. 401 represents the main digital detector board. In the example embodiment shown, board 401 is a PCB using SMD parts. Other example embodiments of the test bench use perf board and through-hole parts or other SMD parts and custom PCB designs for board 401. 402 represents the cable discussed as 205 in FIG. 2. 403 represents the all-in-one digital photodiode which produces a digital output representing the light for MCU 111 to use. Other example embodiments of the digital detector circuit use other digital light and intensity detection methods instead of the all-in-one IC shown in 403.

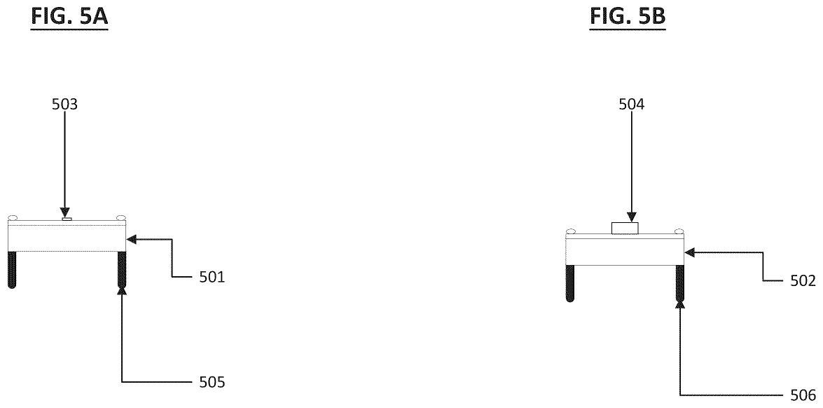

FIG. 5A and FIG. 5B are example embodiments of LEDs and example mounting and connection methods. 501 and 502 represent mounting boards for SMD LEDs. 503 and 504 represent example SMD LEDs of different sizes. 505 and 506 represent the electrical connection pins for the LED mounting systems. Other example embodiments use other sizes and styles of both SMD and through-hole LEDs, as well as other sizes and styles of mounting methods for these LEDs. Multiple sizes of LEDs are tested to determine if LED surface area and light output angle have an effect on the reduction of the melanin bias during testing.

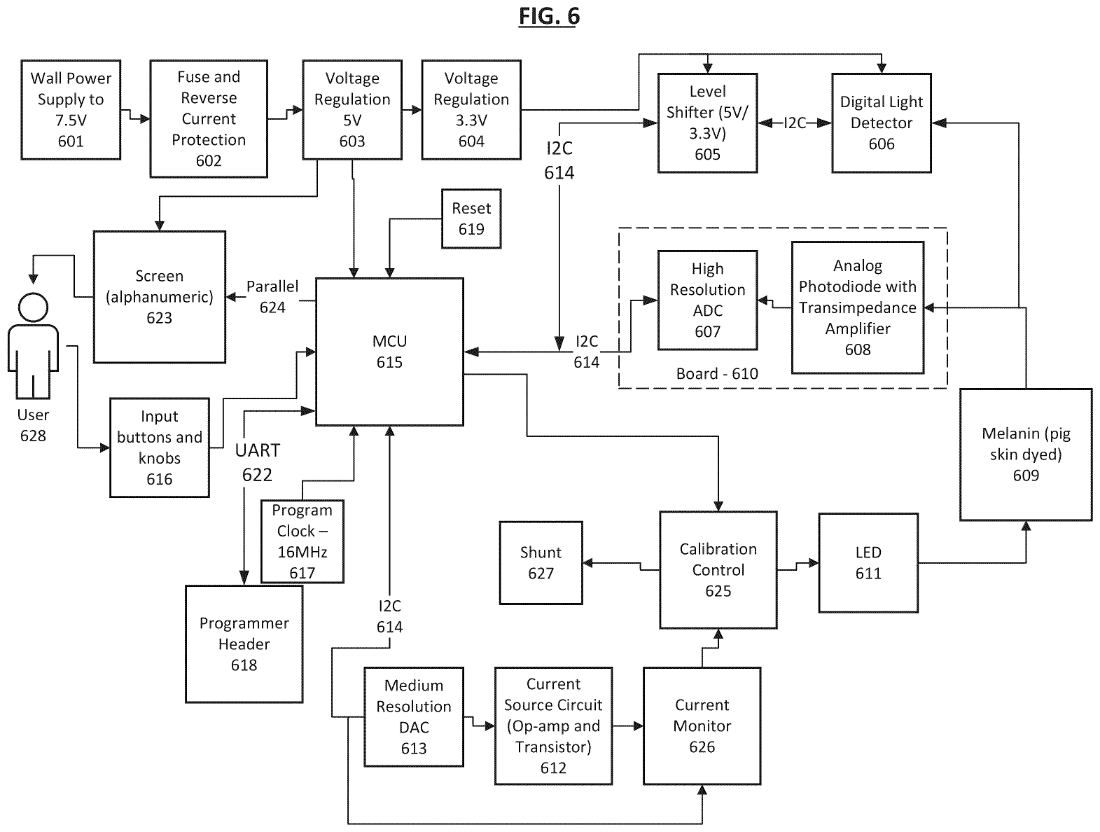

FIG. 6 shows an overview of a general hardware flow of an example embodiment of the test bench system. 601 is the wall power supply that connects through barrel jack 101 to give power to the circuit. Other example embodiments of the test bench system use other power methods instead of wall adaptor 601, such as, but not limited to, mains supply through AC/DC converters, USB power, and/or batteries. Block 602 is the fusing and reverse current protection, also 115 and 128 respectively. In the example embodiment shown, a PTC fuse is used so the device is easily resettable, and a metal-oxide-semiconductor field-effect transistor (MOSFET) with a diode is used for reverse current protection.

Other example embodiments of the test bench system use other styles of fuses, such as, but not limited to, glass or blade fuses and use other forms of reverse current protection, such as, but not limited to, dedicated reverse current protection ICs or single diode methods. 603 and 604 are the 5V and 3.3V regulation blocks respectively, also shown in 116. Other example embodiments of the test bench system use other voltages and/or voltage regulation methods depending on the circuit requirements for those embodiments. 605 is the 5V to 3.3V level shifter for the I²C data stream for the digital detector 606.

Level shifter 605 is also shown in 123. Other example embodiments of the test bench system use other types of level shifters depending on the voltage requirements, and other example embodiments do not require a level shifter for I²C data for digital light detector 606. 606 is the digital light detector, also shown in FIG. 4. In the example embodiment shown, a TSL2591 I²C light detector is used. Other example embodiments of the test bench system use other types of digital light detectors. 607 is the high-resolution ADC, also shown as 303. In the embodiment shown a 16-bit I²C ADS1115 is used. Other example embodiments of the test bench system use other forms of analog converters. It is important that the analog converter has a high enough resolution so that it is able to detect the minute differences between the absorption of oxyhemoglobin and deoxyhemoglobin when transferred for use in the pulse oximeters discussed later in this document. 608 is the analog photodiode, also shown as 304. In the example embodiment shown, an OPT101 was chosen for this part, since it includes a transimpedance amplifier, which makes the overall design require less components. Other example embodiments of the test bench system use other types of analog photodiodes for 608, some of which require an independent transimpedance amplifier circuit. A transimpedance amplifier is required to convert the small currents produced by the photodiode into readable voltages for the ADC 607 to use. In other example embodiments of the test bench system, ADC 607 is internal to MCU 615. 609 is the melanin dyed pig skin. The melanin dyed pig skin is used to simulate different concentrations of melanin in human skin for testing purposes. 610 is the board 301 that both 607 and 608 mount to. 611 is the LED, for example, 503 and 504. During testing many different LEDs are used to find the best option to reduce the melanin bias for the pulse oximeter discussed later in this document. 612 is the op-amp and transistor portion 121 and 120 of the current source circuit. 613 is the DAC 122 portion of the current source circuit. Other example embodiments of the test bench system use other methods to create a digitally controlled variable voltage source for op-amp and transistor 612, such as, but not limited to, PWM with filtering, and/or digital potentiometers. In other example embodiments of the test bench system, DAC 613 is internal to MCU 615. Further, other example embodiments use other methods to produce a controllable current source for the LEDs, such as, but not limited to, current source ICs, digitally controllable regulators, or digitally controllable current source supplies. I²C signal 614 is used to interconnect all of the devices (digital light detector 606, high resolution ADC 607, DAC 613, and current sensor 626) with the MCU 615. MCU 615 (also 111) is where the main program is stored and executes its functions based on user input 628, via buttons and knobs 616 also 104-107. In the embodiment shown MCU 615 is an Atmel ATMEGA 328P microcontroller clocked at 16 MHz, via the crystal oscillator program clock 617(also 112). Other example embodiments of the test bench system use other types of microcontrollers and other types of program clocks running at other speeds. 618 is programming header 113, which is attached to a computer in order to update the program stored in MCU 615, via UART signal 622. Other example embodiments of the test bench system use other programming methods, such as, but not limited to, ICSP, SWD, SPI, J-TAG, and/or wireless methods. Furthermore, other example embodiments of the test bench system use the programming header in order to communicate with a computer or smart device as part of its user interface. 619 is reset button 110. Other example embodiments of the test bench system use a watch dog timer for 619 to automatically reset the MCU in the event of a program hang. 623 is the alpha numeric screen (also 201) portion of the user interface that presents information to user 628, while the test bench system is running. In the example embodiment shown, screen 623 is connected to MCU 615, via parallel interface 624. Other example embodiments of the test bench system use other types of interfaces to connect between MCU 615 and screen 623 such as, for example, SPI, I²C, 1-wire, and other serial methods. Furthermore, other example embodiments of the test bench system use other types of screens for 623, such as, but not limited to, LCD, OLED, LED array, touch screen, and/or computer or smart device interfaces. 625 and 627 (also 119 and 118) are the calibration controller and current shunt, which calibrates the current sensor 626 on initial boot up. This calibration accounts for voltage drift as parts wear out and become less efficient or if different voltage inputs occur due to mains fluctuation. Current sensor 626 (also 117) in the embodiment shown is an INA219 I²C current sensor. In other example embodiments of the test bench system, other current sensors and current sensing methods are implemented. User 628 controls the test bench system, via input buttons and knobs 616 (also 104-107) and receives data back from the test bench system, via screen 623. User 628 also switches out different concentrations of melanin dyed pig skin 609, as well as different LEDs, 611, and detectors, 606 and 608, to test.

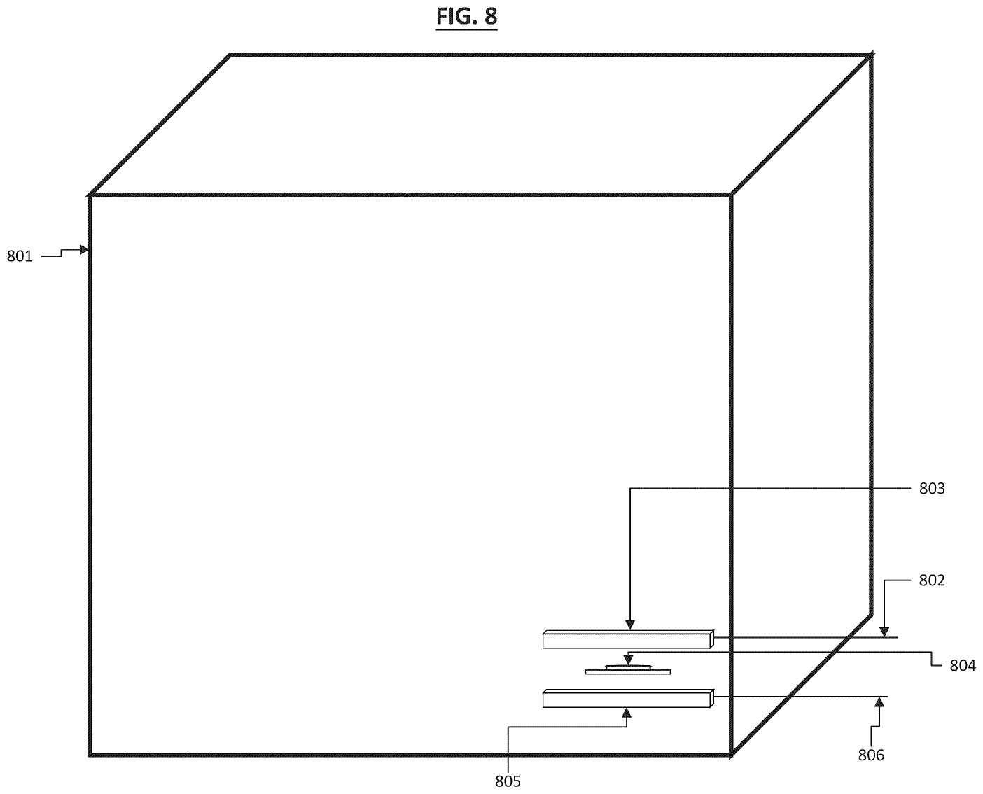

The signal interaction and flow shown, in the example embodiment in FIG. 6, works as follows. User 628 selects the LED 611 to start testing, as well as which detector to start testing with, digital 606 or analog 608. The user 628 places the melanin dyed pig skin 609 between the LED 611 and the detector 606 or 608 and places this setup in a blackout box, 801, to prevent interference from ambient light, as shown in FIG. 8. The user 628 uses input buttons and knobs 616, as well as user interface screen 623 to select menu options, tests, and values to be used in the testing process. Microcontroller 615 clocked, via the program clock 617, runs firmware instructions per user 628's input requests and returns these results to screen 623. Microcontroller 615 controls screen 623, via parallel interface 624, which is controlled by the screen drivers which are part of the firmware on microcontroller 615. Microcontroller 615 controls DAC 613, high resolution ADC 607, current sensor 626, and digital light detector 606, via the I²C bus 614. DAC 613's output voltage is sent to op-amp and transistor 612, configured in comparator mode to create a current source that controls LED 611, and is monitored by current sensor 626. Light from LED 611 passes through pig skin 609 and enters either a digital light detector 606 or analog photodiode 608 depending on user 628's input selection. Analog photodiode 608 includes a built in transimpedance amplifier in the example embodiment shown, which sends its voltage output signal to ADC 607 to return a digital intensity value to MCU 615. Other example embodiments use phototransistors instead of photodiode 608. Further, other example embodiments use, for example, an analog photodiode 608 with external transimpedance amplifier circuits. If user 628 selects a digital input method, digital light detector 606 sends its digital intensity data to microcontroller 615 over I²C bus 614, via level shifter 605. Other example embodiments use other digital light detection methods and communication methods instead of digital detector 606. Microcontroller 615 controls calibration circuit 625, which when enabled, diverts current from LED 611 to current shunt 627, which allows for known current values to be returned based on the fixed resistor value of the current shunt 627, in order to calibrate DAC 613 and current source circuit 612. Programming header 618 is used to program/update firmware on MCU 615 over UART interface 622. Power for all circuits is generated via 5V regulation 603 which receives its power from wall supply 601 via fuse and reverse current protection 602. Light detector 606 requires voltage level shifting 605 and receives its power, via 3.3V regulation 604, which receives its power, via 5V regulation 603. Other example embodiments of hardware flow are used in other embodiments of the test bench system, in accordance with the invention, via other example signal paths and circuits.

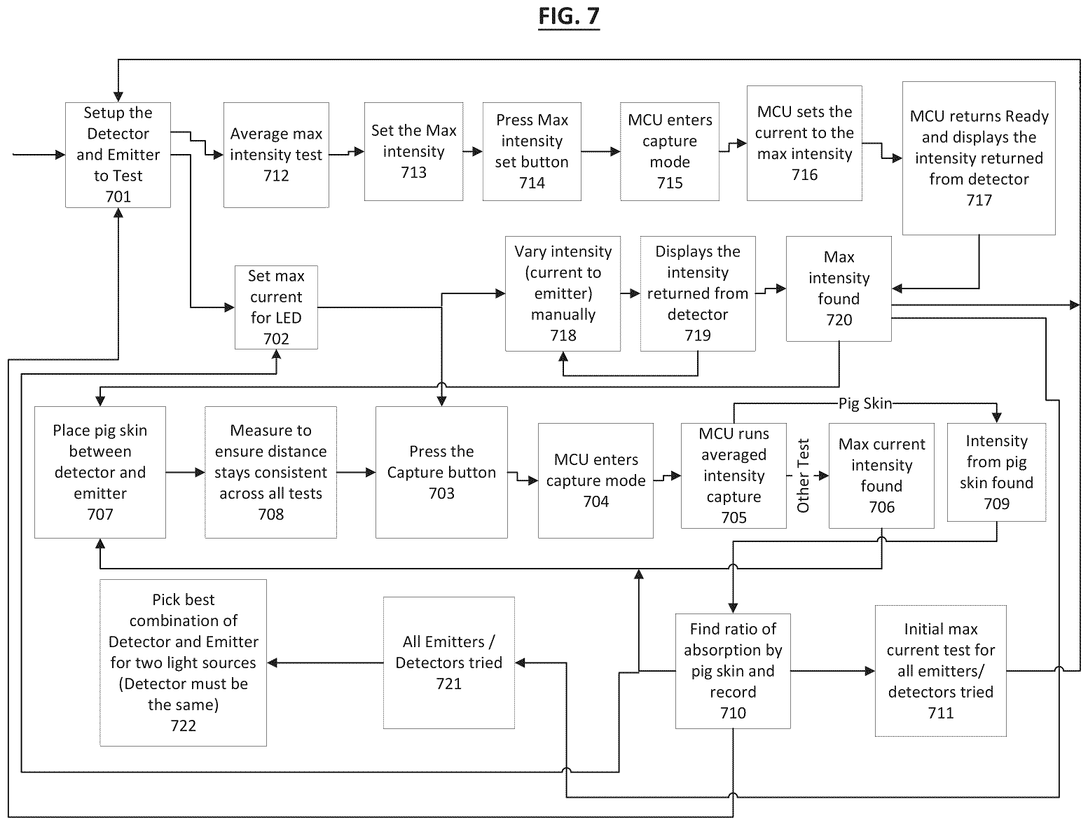

FIG. 7 shows an example of a general usage of an example embodiment of a test bench used to find the best LED and detector combination for a method of measuring blood oxygen saturation that reduces the melanin bias, via an example user interface. In block 701, the user sets up the detector and emitter to start testing with, as shown in FIG. 8. The user starts the example usage process by setting the max current for the LED 702. The user presses the capture button 703, in order to begin the max current test, which is used to determine the maximum returned intensity for all emitters to have a comparison point. The MCU (also 111 and 615) enters capture mode 704. After capturing the intensity from the detector, the intensity values across the time span of the capture are averaged in block 705. Max current intensity found is returned to block 706. The user, following the example diagram in FIG. 8, places the pig skin between the detector and emitter 707. The user measures the distance, as described in FIG. 8, to ensure the distance stays consistent in all tests in block 708. The user performs blocks 703 through 705, at which point, the intensity from the pig skin is found in block 709. The user finds the absorption ratio of the pig skin and records this value in block 710. Steps 701 through 710 are repeated with all varying degrees of melanin and all emitters and detectors until the max current test for all combinations is tried in block 711. The user again begins with the initial detector and emitter in 701 and runs the average max intensity test, 712 by setting the max intensity in block 713, found during the previous tests. The user presses the max intensity set button 714, which places the microcontroller into capture mode 715 and calculates the current required to achieve this maximum intensity in block 716. The MCU returns the actual intensity the emitter is capable of with the intensity and current parameters given in block 717. At this point, the max intensity is found and is recorded by the user in block 720 before continuing with steps 707 through 710 to find the maximum intensity and pig skin absorption ratios for all detector and emitter combinations. In order to verify that there are no peak or trough anomalies across the current values for each emitter and detector combination, the user performs a manual current test by setting up the initial detector and emitter in 701. The user sets the max current for this emitter in 702 and varies the current of the emitter manually in 718, recording any unexpected peaks or troughs in intensity displayed in block 719. Once the max intensity peak is found during the manual current test in block 720, the process for manual current testing in block 701, 702, and 718-720 are repeated until all emitter and detector combinations are tried. At this point, the user moves to block 721 and reviews the data, in order to pick the best emitter and detector for a method of measuring blood oxygen saturation that reduces the melanin bias in 722. In the example embodiment shown, the best two emitters are chosen for reasons discussed in other figures of this document, and only one detector is chosen. Other example embodiments of the test bench system, use other tests, user interface flows, and procedures, in order to produce the same end result of choosing the best emitters and detector combination for a method of measuring blood oxygen saturation that reduces the melanin bias.

FIG. 8 shows a general usage method of the test bench using, for example, pig skin and an example black-out box. During testing, described previously in the document, in order to prevent ambient light and other light sources to interfere with test results, the following is an example embodiment of usage in which black-out box 801 includes the optical testing portion of the test bench system. 805 represents the detector portion, which is either digital light detector 606 (also 203) or analog photodiode 608 (also 204). 803 represents LED 611. 804 represents pig skin 609 placed on a slide, and as shown, pig skin 804 and the slide is placed between 803 and 805 and held at an even spacing, in order to keep the same distance between the LED and the detector throughout the testing process. 806 represents the cable from detector 805(also cable 205/402 or cable 206/302), in which cable 806 connects to the test bench board 129/202. Cable 802 (also cable 126 or 127) connects LED 803 to the test bench board 129/202. During the usage, test bench board 129/202 is kept outside the black-out box, 801, in order to give users access to the user interface, as well as to prevent the user interface from causing light interference.

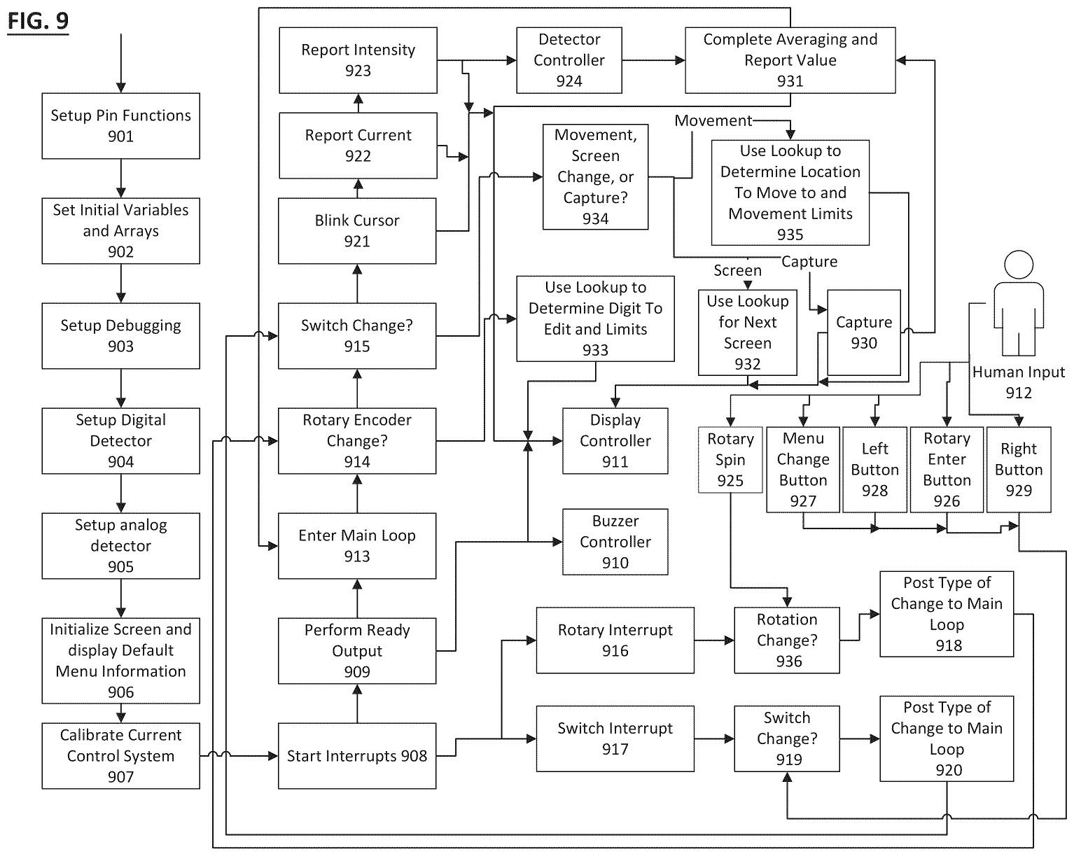

FIG. 9 shows an example of a general software flow of an example embodiment of a test bench system. The program runs on MCU 111 (also 615) and enters at block 901, which sets up all pin functions for the MCU and peripheral hardware. The program sets up the initial values for variables and arrays in block 902 and starts its debugging interface in block 903. The system sets up the digital detector in 904 and the analog detector in 905. The screen is initialized and default menu information posted in block 906. The system calibrates its current control system in block 907 and starts its interrupts in block 908. The interrupts on this system, include, for example, rotary interrupt 916, which senses rotation changes in rotary encoder 106, and switch interrupt 917, which detects switch presses from 104 through 107. The system performs its system ready output in block 909, via display controller 911 and buzzer controller 910, before entering the main loop 913. Main loop 913 begins by checking if a rotary encoder change flag exists in block 914, and checks if a switch change flag exists in 915. The main loop blinks the cursor in 921 before reporting the current in 922 and the intensity in 923. The intensity is reported in 923, via the detector controller 924. Detector controller 924 is used to select which detector the user is currently requesting and sends its intensity data to block 931, where averaging and value reporting are done, before going back to the beginning of the loop 913. Buzzer controller 910 determines the duration of buzzer signaling and when in the program to signal the buzzer. Display controller 911 is responsible for mathematically determining positions on the screen for data output and user input to be displayed, as well as handling the physical parallel interface 624 to the screen 623. If a rotary change flag is detected in 914, look up tables are used to determine the digit to edit and its limits in both directions in block 933 before sending this data to display controller 911. If a switch change flag is detected in 915, the type of change, depending on which button, is determined in 934. The type of changes in 934 are movement, screen change, or capture commands. If a movement command is detected, a look up table is used to determine the location to move to and the movement limits in 935, before sending this data to display controller 911. If a screen change is requested, a look up table is used to determine the next screen to display in 932, before sending this data to display controller 911. If a capture command is requested, the capture begins in 930 as outlined in FIG. 7 before returning data to display controller 911 directly or completing averaging in 931. 921 through 923, as well as 931, all send data to display controller 911 for display on screen 623. The user (human input 912) interacts with the user interface, via rotary spin 925, menu change button 927, left button 928, right button 929, and rotary enter button 926. Rotary spin 925 sets rotary change 936 in rotary interrupt 916 and posts a rotary change 918 to the main loop. Buttons 926 through 929 set a switch change 919 in switch interrupt 917 and posts a switch change 920 to the main loop. The main loop 913 runs until the system is powered off, via the user, reset button 110, or the watch dog software system. Other example embodiments of the test bench software use other methods, block ordering, and flow, in order to produce the same result for detectors and emitters in accordance with the invention.

### Pulse Oximeter System

As previously mentioned, current pulse oximeters use an optical arrangement that is biased against those with darker skin or high concentrations of melanin (naturally, from disease, or from suntan). This bias often causes falsely high blood oxygen saturation levels (SpO₂) which often results in a high frequency of hidden hypoxemia, high disparity of care, and poor medical outcomes for these individuals. Current pulse oximeters also do not function well on patients wearing nail polish, those with poor perfusion (for example an individual with Raynaud's syndrome, etc.), those with amputations, and those with aversions to wearing devices on their fingertips. Current pulse oximeters are also affected by motion artifacts, are uncomfortable to wear, are hard to use on infants, and they reduce the wearer's ability to use both hands. In these situations, current pulse oximeter readings are affected in different ways which provide false or inaccurate readings. The melanin bias issue and nail polish problem often cause a falsely high reading or no reading (depending on nail polish thickness) due to the absorption and scattering of current pulse oximeter optics. Motion and poor perfusion often cause problems with obtaining a valid pleth graph and affect the oxygen saturation readings in various ways. The pulse oximeter described in this document reduces the melanin bias and the other issues with current pulse oximeters using the methods and embodiments described herein.

Pulse oximeters use two or more wavelengths of light to detect the levels of absorption of oxyhemoglobin and deoxyhemoglobin. The wavelengths chosen are on opposite sides of an isosbestic point of oxyhemoglobin and deoxyhemoglobin, which provides the means to use the Beer-Lambert law and the following equation to determine the output R value or ratio value of oxyhemoglobin and deoxyhemoglobin.

$$R = \frac{( A {C}_{rm s} LED 1 / D C LED 1 )}{( A {C}_{rm s} LED 2 / D C LED 2 )}$$

The R output is used against a lookup table to correlate the R to the calibrated SpO₂. The lookup table is generated during a calibration study or trial of the device in real world conditions using a large patient population of subjects. It is important to obtain subjects with a wide range of skin tones, age, and medical status to ensure a well calibrated device is produced. Calibration is performed in a medical setting by correlating the R value to the blood oxygen saturation reported using the gold standard of arterial blood gas testing. Pulse oximetry is preferred over arterial blood gas testing, as pulse oximetry is non-invasive, provides more than a snapshot oxygen saturation, and is a pain free measurement method. Pulse oximetry provides the percentage of oxygen in the blood and is an important vital sign, as it often guides decisions about starting oxygen therapy. Using the previous description of blood oxygen saturation (SpO₂) when referring to the ratio of oxyhemoglobin and deoxyhemoglobin, the following equation is applied to pulse oximetry.

$${SpO}_{2} = ( \frac{Oxyhemoglobin}{( Oxyhemoglobin + Deoxyhemoglobin )} ) \times 100 = ( \frac{OxyHb}{( OxyHb + DeoxyHb )} ) \times 100$$

Additionally, one example of an equation to calibrate SpO₂ based on lookup table information is:

SpO₂=aR²+bR+c where R is the ratio as described above and a, b, and c are look up values or predetermined calibration values from statistical data collected during calibration studies.

Pulse oximeters also produce a plethysmograph (pleth graph) which shows the perfusion of blood volume in the extremity the device is placed on. The pleth graph is often produced using only one LED's (emitter) received data, and in some example embodiments the pleth graph is a combination average of all the LEDs' (emitters) received data. The pleth graph changes with each heart beat as a volume of oxygenated blood flows in and out of the appendage. The pleth graph is a useful tool in determining if the oxygen saturation readings resulted are being distorted by poor perfusion, noise artifacts, or motion artifacts. The pleth graph is also used to calculate the subject's heart rate by reading the time between peaks of the pleth graph, since each peak is relative to the beat of the subject's heart.

Current pulse oximeters also include inherit problems with LED (emitter) wavelength drift due to, for example, aging, manufacturing tolerance, temperature, voltage fluctuations, and other sources of error variables. These drifts when utilizing an R value (ratio) calibrated SpO₂ method as previously discussed often leads to further significant errors in SpO₂. These drifts are accounted for in some example embodiments by using highly calibrated and tolerant LEDs (emitters) in the design process. Other example embodiments place an expiration date on the pulse oximeter to combat age related drift. Further, other example embodiments use temperature sensors embedded near or within the emitters to further calibrate the wavelength offset in real-time based on a known wavelength deflection vs temperature curve. Further embodiments utilize feedback loops for on-board calibration, and other embodiments utilize more than two emitters to produce an averaged value. Further example embodiments utilize a calibration free method by utilizing emitters (either LED or laser) with narrow spectral line-width. In order to achieve a calibration free design these embodiments often utilize relatively nearby wavelengths for calculating the SpO₂. Further example embodiments utilize polarized light in order to increase accuracy.

Further embodiments utilize a single LED wavelength to generate SpO₂ through the means of a LED-based vector-beam. The vector beam is fully polarized but shows different polarization states in different local positions in the same detector plane of vision to provide the means required to produce SpO₂ readings. Further example embodiments utilize a variable gradient or optical filter to electrically adjust the wavelength of a single LED (emitter). Examples of such embodiments adjust the wavelength of single LED to achieve a melanin bias reducing wavelength. Further example embodiments utilize an electrically tunable emitter to adjust the wavelength as required with a single emitter. Additionally, further example embodiments utilize a single LED (emitter) in conjunction with a multispectral sensor such as for example, a buried quad junction (BQJ) photodetector where the oxyhemoglobin and deoxyhemoglobin act as an optical filter to the emitter's light. The BQJ detector distinguishes the changes in the light caused by the optical filtering of the two types hemoglobin and this change in the light spectrum is used to calculate the SpO₂ levels. In a BQJ detector embodiment the multiple channels of the BQJ are used in a ratio and for creating a pleth graph and heart rate with a single LED wavelength rather than using multiple timed captures with a single output photodetector and two or more emitter wavelengths that are independently controlled.

The following describes a two emitter and single channel photodetector example embodiment that utilizes the information determined through invitro testing to reduce the melanin bias found in current pulse oximeters. To achieve the melanin bias reduction optical hardware modifications to the wavelengths are used and software is used to provide additional anti-scattering feedback loops. The detailed descriptions of the drawings utilizing a two emitter and single channel photodetector are one example embodiment implementation and other aspects and methods of pulse oximeter design, as described above are implemented in further example embodiments combining aspects of the melanin bias reducing hardware described below.

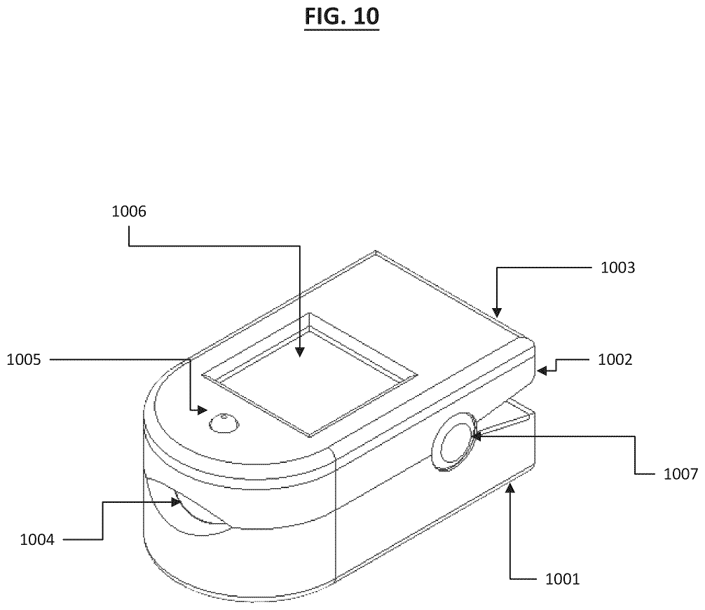

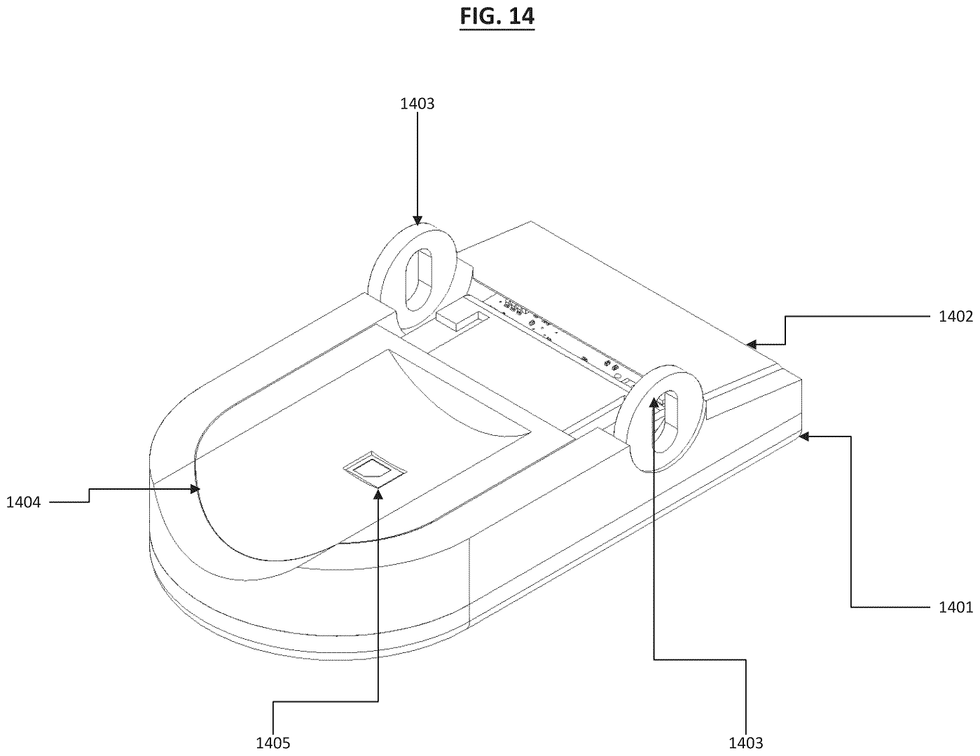

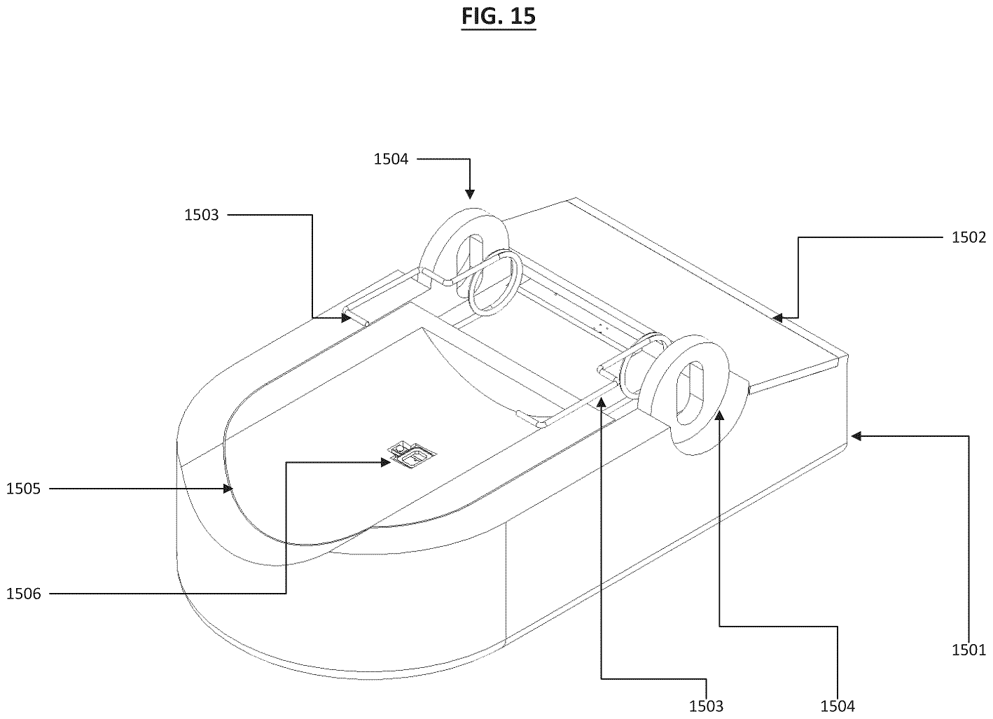

FIG. 10 is a diagram of an example embodiment of a finger clip design of a pulse oximeter. The example embodiment shown in FIG. 10 includes a built-in user interface, including a built-in screen and button. Other example embodiments of the finger clip design, discussed later in this document, do not include a built-in user interface, but rather connect to a patient monitoring system, smart device, or other external user interface. Further, other example embodiments use other input methods for the UI such as, for example, touch screens, capacitive touch, capacitive auto finger detection, and other interface methods. 1001 is the bottom half (or lower half) of the pulse oximeter, further described in FIG. 15. 1002 is the upper half (top half) of the pulse oximeter, further described in FIG. 14, also including upper lid (top lid) 1003. 1003 is the top lid of the pulse oximeter, which includes the cut-out for the screen portion 1006 of the user interface. 1005 is the user button portion of the user interface, which protrudes from 1003. 1006 includes a clear protective cover attached to 1003, in order to protect the screen. As mentioned above other example embodiments utilize other UI methods and therefore do not include the cut-out for screen portion 1006 or user button 1005. 1004 is the cut-out in 1001 and 1002, which allows the user finger to be placed in the pulse oximeter. 1007 is the hinge pin that holds 1001 and 1002 together, as well as allows the finger clip to perform in a clamping fashion around the user's finger. The hinge 1007 is designed in such a fashion to allow the lower half, 1001, and the upper half, 1002, to shift to expand finger opening, 1004, to accommodate different sized fingers. The demonstrated embodiment is made out of plastic with rubber inserts, as well as metal springs, wherein the plastic is ABS, PET, and or PLA, and the rubber is primarily TPU. Although the demonstrated example embodiment is made out of primarily plastic with rubber inserts, the meter may take on other forms and shapes using other materials, such as, but not limited to, rubber, foam, vinyl, medical grade materials, antimicrobial materials, and/or metal.

The embodiment described in FIG. 10 primarily describes an example standalone pulse oximetry finger clip embodiment with a self-contained power source displaying its data on an internal screen. A further example embodiment includes a small low power screen for example e-ink or OLED to display pulse oximetry data directly on the finger clip. Further example embodiments do not include a built-in screen and include an interface to connect to a wired screen or patient monitoring system.

Further embodiments do not include a built-in screen and include an interface to connect wirelessly to a screen, patient monitor system, smart device, or other embodiment described in the additional embodiments section of this document. Further embodiments include both a built-in screen and include an interface to connect wirelessly to a screen, patient monitor system, smart device, or other embodiment described in the additional embodiments section of this document. Further example embodiments provide an interface to wirelessly transmit pulse oximetry data to a physician office as an Internet of Things device, or via a smart device connection. Further example embodiments utilize reduced component counts in the finger clip to connect to an external screen or patient monitoring system in a similar fashion as current pulse oximeters. These embodiments provide a solution to an inexpensive disposable finger clip, finger stall, or adhesive finger pulse oximeter, and only include, for example, the emitter and detector. Further an example of a reduced component embodiment which connects to a patient monitoring system similar to current pulse oximeters include, for example, only the LED emitters, which is controlled by the system it is connected to or in some example embodiments includes the LED emitters with minimal constant current control and switching logic. Further an example of a reduced component embodiment which connects to a patient monitoring system similar to current pulse oximeters include, for example, only the photodiode and transmits the small current signals directly to the system it is connected to. However, this embodiment is highly susceptible to noise due to the transmission of small current signals, and therefore often requires extensive shielding. Further an example of a reduced component embodiment which connects to a patient monitoring system similar to current pulse oximeters includes, for example, the photodiode with a transimpedance stage to directly send the analog voltage signals to the system it is connected to. However, this embodiment is also susceptible to noise due to the transmission of analog signals, and therefore often requires extensive shielding. Further an example of a reduced component embodiment which connects to a patient monitoring system similar to current pulse oximeters includes, for example, the photodiode with a transimpedance stage and digital conversion stage to directly send the digital intensity signals to the system it is connected to. These example embodiments represent a drop-in replacement for current pulse oximeters with the included melanin bias reduction optical hardware described in this document.

In other words, these example embodiments are comparable in component count and price to current disposable pulse oximeters and function with the same electrical connections to current patient monitoring systems while including the additional melanin bias reduction functionality. Additionally, example embodiments include motion and location tracking capabilities, as further described in the ring pulse oximeter section of this document to further reduce the number of monitoring devices required for a subject.

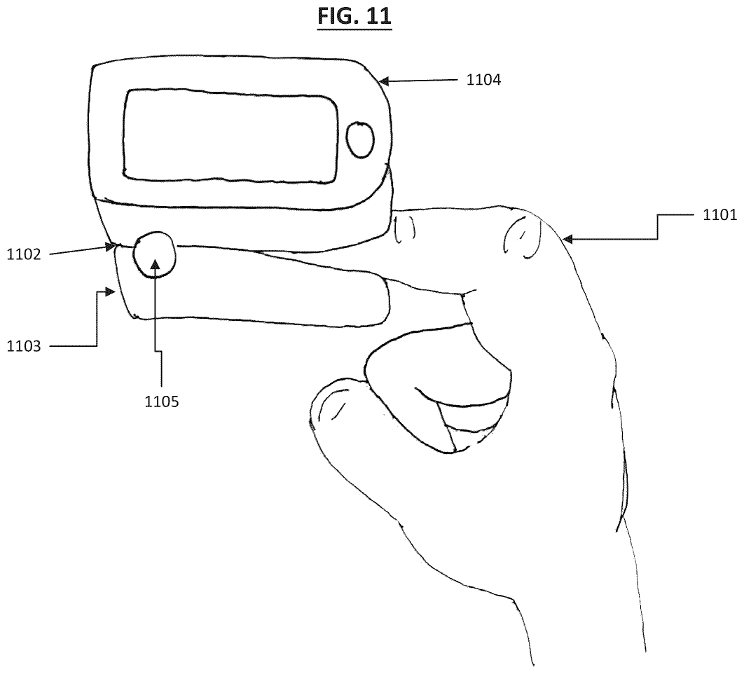

FIG. 11 shows a diagram of an example embodiment of a finger clip design of a pulse oximeter, as previously shown in FIG. 10 with added finger placement. 1101 shows the finger being inserted into finger clamp 1102 between upper portion 1104 (also 1002) and bottom portion 1103 (also 1001). Pivot hinge 1105 (also 1007) is designed to move to allow 1103 and 1104 to dynamically shift, in order to allow finger 1101 to fit snugly between 1103 and 1104 without injuring finger 1101. It is important that finger 1101 fits snugly so that the optics (described later in this document) required for pulse oximetry and included within 1103 and 1104 are held firmly against finger 1101. The snug fit allows for accurate pulse oximetry calculations, as well as prevents artifacts from finger 1101 movement to interfere with the pulse oximetry reading.

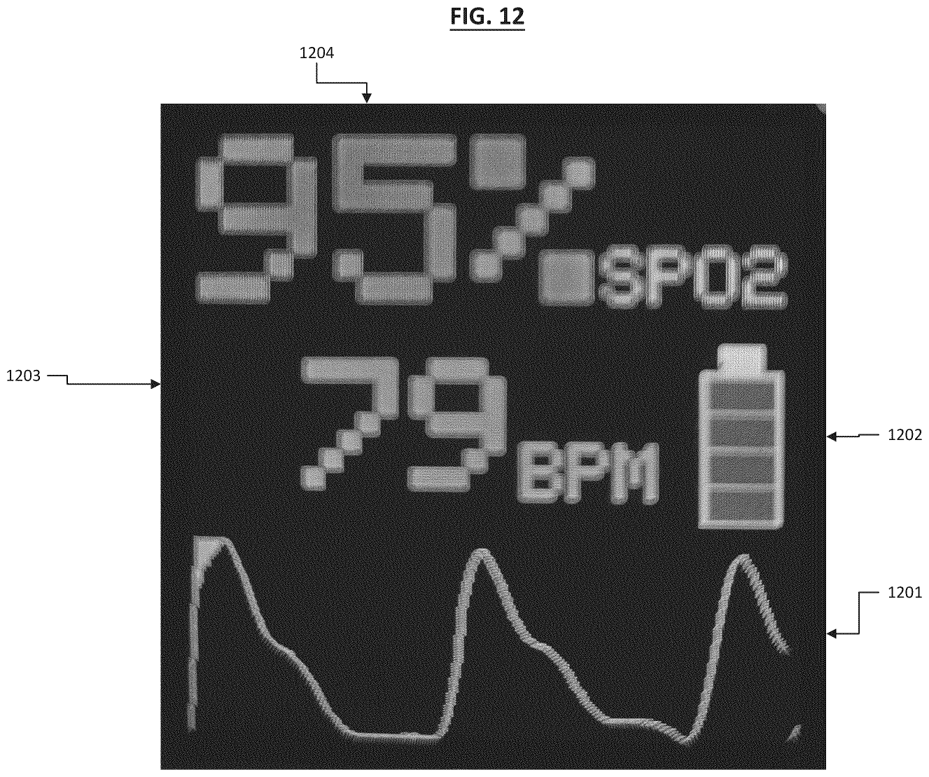

FIG. 12 is an example embodiment of a general lay-out of a user interface for a pulse oximetry system. The user interface described in FIG. 12 is displayed on the screen portion of the user interface. 1201 is the pleth graph, which is used to determine if the meter is getting a good reading, as well as measuring and showing the changes in blood volume, or perfusion changes, in the area that the meter is attached. A good reading is determined by a regular, non-weak signal pleth graph. The pleth graph is also used to determine heart rate based on the peaks of the graph. It is important to note that if the patient's natural heart rate is irregular, the pleth graph will appear irregular as well, even if the meter is getting a good reading. The procedure for pleth graph interpretation is discussed further in the firmware portion of this document. 1202 represents the battery indicator, which is used to determine the amount of remaining battery left in portable meters. One example embodiment uses a segmented meter to demonstrate the amount of remaining battery life, as well as colors to visually indicate good, medium, or critical battery life. 1203 represents the beats/minute of the patient's heart rate. 1204 represents the percentage of blood oxygen saturation (SPO2). 1204 changes color depending on the patient's oxygen level to more easily demonstrate a good (95-100%), moderate (90-95%), or critical (<90%) oxygen level for a layperson. The numbers and text on the screen utilize scalable number, text, and symbol character maps stored in the firmware to accommodate multiple sizes of screens. Other example embodiments of the pulse oximetry system use other styles, arrangements, and data sets to make up the information displayed on their user interface depending on the application required. In systems that are plugged into mains power, it is important that they include a backup battery, since pulse oximetry blood oxygen saturation levels and heart rate are often considered critical patient care infrastructure. In the case of a wall powered system, 1202 will represent the backup battery level and during mains powered operation, it will display either a charging indicator or a mains power symbol. In other example embodiments, described further in this document, that require full ECG interfaces or full patient monitoring interfaces, such as embodiments that include both pulse oximetry and heart monitoring chest electrodes, the user interface described in FIG. 12 will also include the appropriate required heart rate graphs, ECG, and other monitoring information, such as but not limited to, respiratory rate, BP, temperature, and CO2. Further, other embodiments, for example ring and flexible bracelet embodiments, include additional hardware which requires the UI to display, for example, but not limited to, blood sugar and other blood gas information.

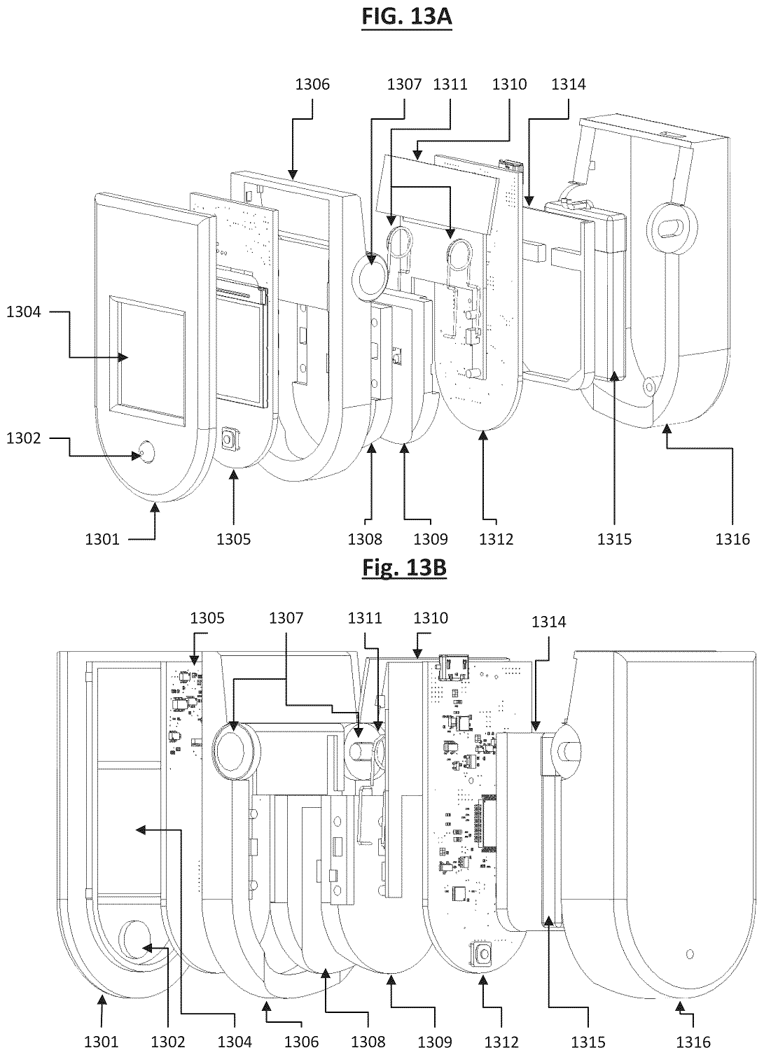

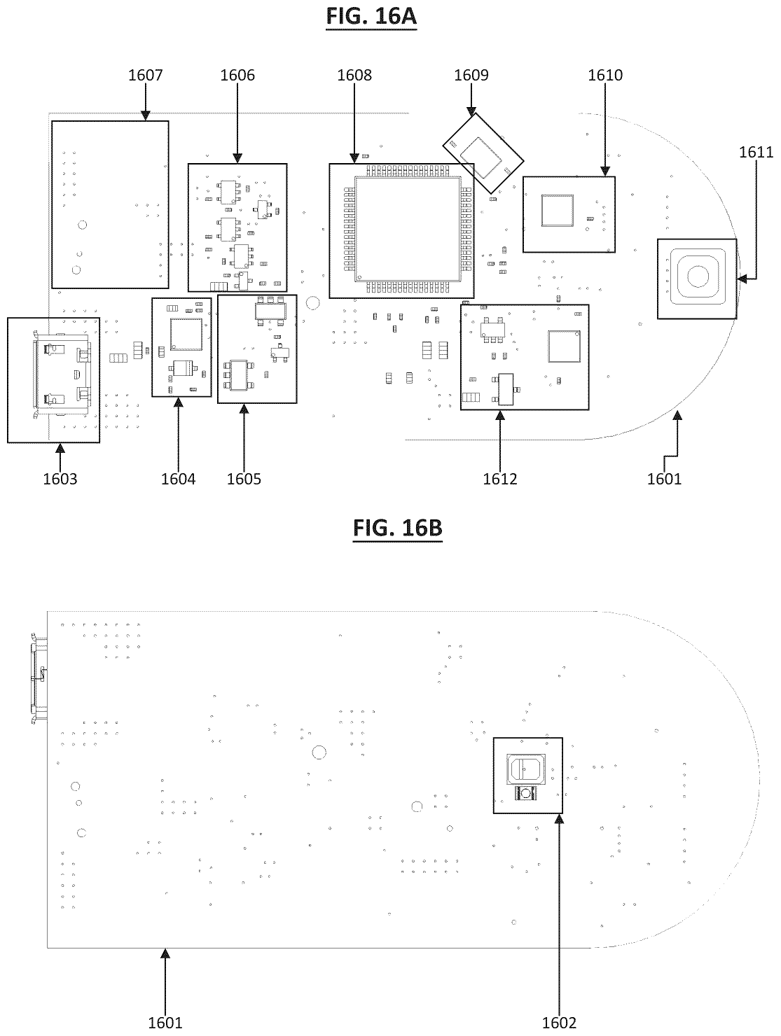

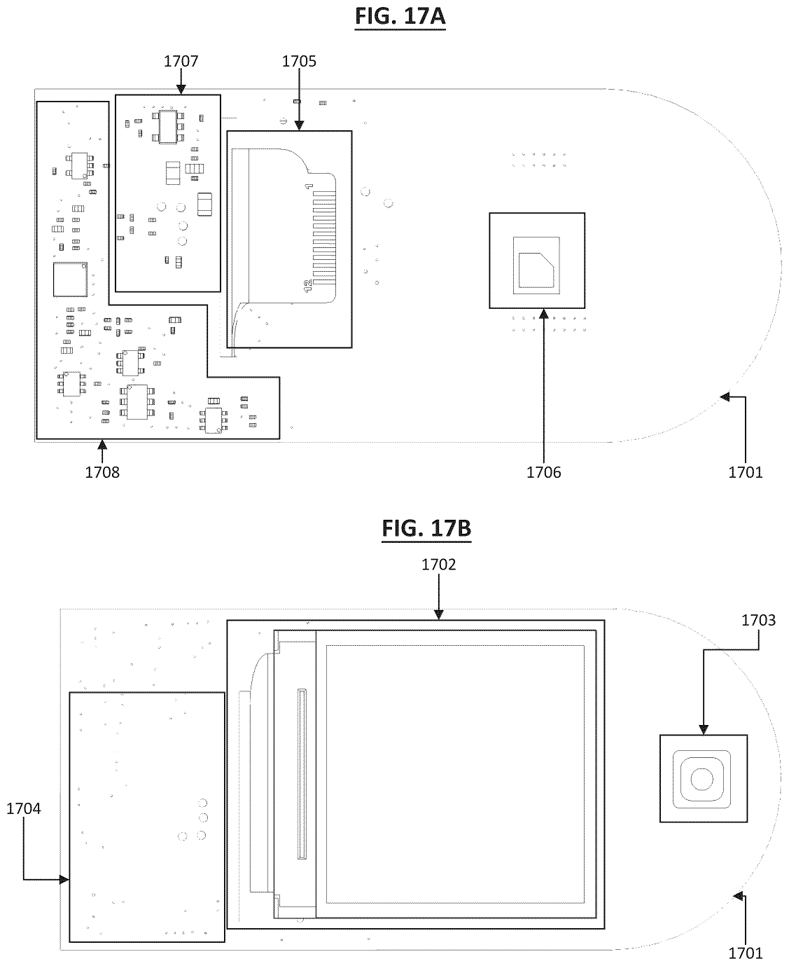

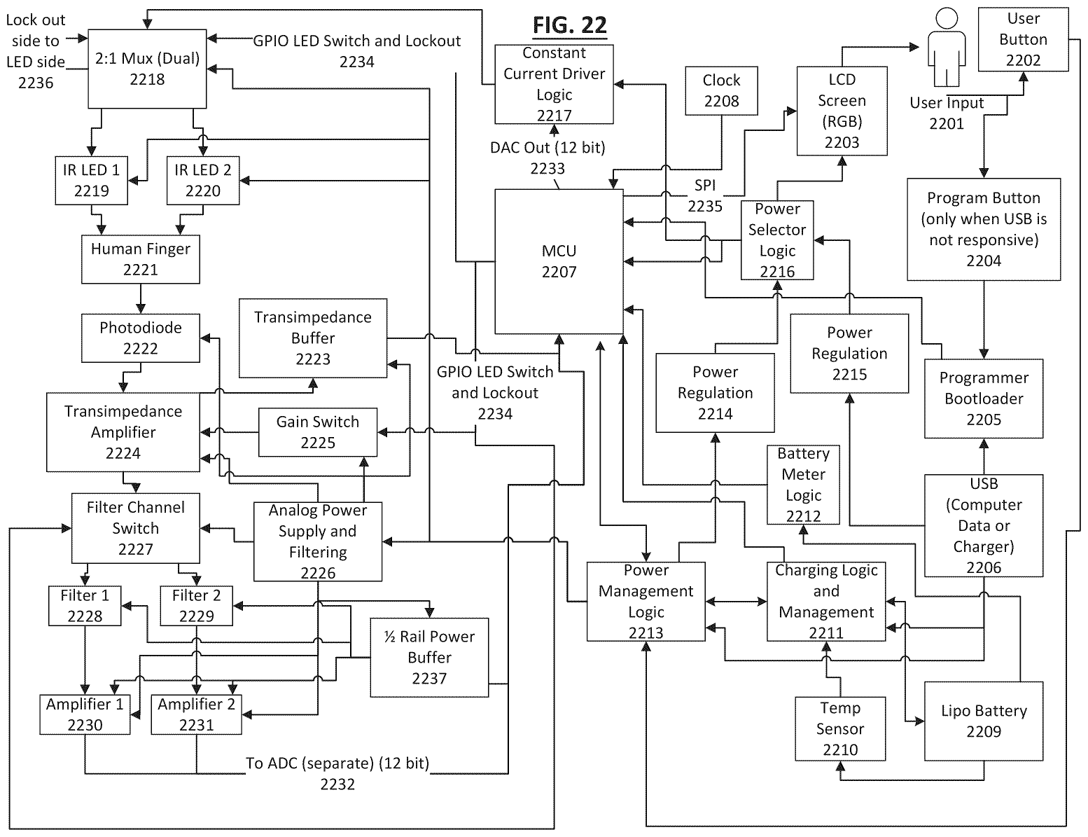

FIGS. 13 A and B show an example exploded view of both the front side view (FIG. 13A) and rear side view (FIG. 13B) of an example embodiment of a finger clip design of a pulse oximeter which reduces the melanin bias. 1301 (also 1003) is the top lid, which includes the cut-out 1304 and protector shield (also 1006) for the screen mounted on the circuit board 1305. 1302 is the user button extension, also part of the top lid 1301, and shown as 1005. Extension 1302 presses the user button, also mounted on circuit board 1305. Upper circuit board 1305, in the example embodiment, is a double layer circuit board, as shown in FIGS. 13A and 13B, where 1305 has different components on both sides. 1306 is the upper case, also shown as 1002. 1306 houses and protects circuit board 1305 and also includes the rubber finger pad 1308, which protects the user's finger from the circuit underneath, as well as gives a comfortable area for the finger to rest. The detector on circuit board 1305 is exposed through a cut-out in 1308 allowing light to pass through the user's finger into the face of the detector. In some example embodiments, 1308 has a clear protector in the cut-out to protect the detector. 1307 (also 1007) are the two hinge pins that connect the upper case 1306 and the lower case 1316 and allow the case to pivot and expand in such a fashion to hold the user's finger. Other example embodiments are designed, for example, with extended sections behind the hinge pins 1307 and/or modified hinge designs to increase the ease of actuating the clip for patients who are elderly and/or others who have problems with hand use and coordination. Further, other example embodiments are designed, for example, with shortened sections behind the hinge pins 1307 and/or modified hinge designs to childproof the design and decrease the ease of actuating the clip for children. 1309 is the lower rubber insert that protects the user's finger from the circuit underneath, as well as giving a comfortable area for the finger to rest, similar to 1308. The LED, or in some embodiments the LEDs (discussed later in this document), on circuit board 1312 is exposed through a cut-out on 1309 allowing light to leave the LED face passing through the user's finger. In some example embodiments, 1309 has a clear protector in the cut-out to protect the LED/LEDs. 1310 is the circuit board protector, which covers the top part of the circuit board 1312 and gives a space for the ribbon cables (not shown in this diagram) to pass, in order to connect circuit boards 1305 and 1312. 1311 are the hinge springs that connect the upper and lower part of the pulse oximeter together and give the pulse oximeter its clamping force to securely attach to a finger. 1312 is the lower circuit board, where in the example embodiment, it is a double layer circuit board shown in FIGS. 13A and B, where it has different components on both sides. 1314 is the battery support cover, which spaces the battery 1315 appropriately below circuit board 1312 to prevent the battery from touching any of the integrated circuits and other components on circuit board 1312. 1315 is the battery, which in the example embodiment shown, is a lithium polymer 3.7V 650 mAh rechargeable battery. Other example embodiments of the pulse oximetry system use other example types of batteries as further described in FIG. 22. 1316 is the lower part of the pulse oximeter case, which houses 1315, 1314, and 1312, and allows 1310, 1311, and 1309 to attach to it. Other example embodiments of 1305 and 1312 use other types of circuit boards, designs, layouts, and components other than those described in the embodiment shown, further described in FIG. 16 and FIG. 17. Other example embodiments use other mechanical arrangements, part orderings, and aesthetic shapes than those shown in FIGS. 13A and 13B. Further, other example embodiments place the emitter and detector in other positions in the case including, but not limited to, opposite sides of the appendage. Further, other example embodiments in the pulse oximeter system do not include the UI components shown in FIGS. 13A and 13B on the pulse oximeter finger clip itself. Instead, in these other example embodiments, these UI components are located in other areas, for example, but not limited to, smart devices, external interfaces, bedside monitoring, and/or patient monitoring systems. Further, other example embodiments use reflective technology where the light from the LEDs enter the human subject/patient and is reflected back to the detector on the same plane as the LEDs. In these example embodiments both the detector and emitter are placed on the same board, which is either 1305 or 1312 in the example shown.

FIG. 14 shows an assembled view of an upper (top) half of an example embodiment of a finger clip design of a pulse oximeter, also shown in FIG. 10 and FIG. 13. 1401 is the top lid of the pulse oximeter, also 1003 and 1301. 1402 is the top case of the pulse oximeter, also 1002 and 1306. 1403 is the top half of the hinge that mates with the bottom half of the hinge 1504, and in which hinge pin 1307(1007) goes through to connect the upper and lower half together. 1404 is the rubber finger protector, also 1308 previously described in FIG. 13, and is made of TPU rubber in the example shown. Other example embodiments use other comfortable materials for 1404, such as, but not limited to, EVA foam, other rubbers, medical grade materials, and/or antimicrobial materials. 1405 is the detector, which goes through 1404 to detect the intensity of the light returned after passing through the subject's/patient's finger. 1405 is mounted to circuit board 1305 and is previously described in FIG. 13. Other example embodiments use reflective methods and use other mounting locations for 1405, as described above.

FIG. 15 shows an assembled view of a lower (bottom) half of an example embodiment of a finger clip design of the pulse oximeter. 1501 is the lower case as described in 1316 and 1001. 1502 is the circuit board protector, also described in 1310. 1503 are the springs, also described in 1311. 1504 is the bottom half of the hinge which mates with 1403, which hinge pin 1307 goes through. 1505 is the rubber finger protector, also described in FIG. 13 as 1309, which protects circuit board 1312. 1506 is the LED/LEDs which goes through 1505 to emit light to pass through the subject's/patient's finger. 1506 is mounted to circuit board 1312 and is previously described in FIG. 13. Other example embodiments use reflective methods and use other mounting locations for 1506, as described above.

FIGS. 16A and 16B show a front and back view of an example embodiment of an example layout of the lower melanin bias reducing pulse oximeter circuit board, also shown as 1312. 1601 is an example embodiment of a PCB which includes the traces and pads for the components. 1602 shows the LED block which includes the LEDs, also 1506, further described in FIG. 22. Other example embodiments use laser light emission methods with the accompanied hardware for 1602. 1603 shows the USB charging connector. In the example embodiment shown, USB connector 1603 is also used as a programming port connected to programmer block 1610. In some example commercial embodiments, the programming feature of 1603 is removed to prevent user access to firmware. In other example embodiments, 1603 is replaced with other charging port styles, such as, but not limited to, barrel jacks, Molex connectors, wireless charging, QI charging, inductive charging, and/or proprietary charging ports. In other example embodiments 1603 is a connector for a patient monitoring system link. 1604 is the charging block, which interfaces between charging port 1603 and battery 1315. In other example embodiments, charging block 1604, as well as charging port 1603, are replaced with a mains power circuit and battery backup controller. Further, in other example embodiments, charging block 1604, as well as charging port 1603, are replaced with a wireless charging method and wireless charging controllers. 1605 is the voltage regulation block, further described in FIG. 22. 1606 is the power management logic block, further described in FIG. 22. 1607 are the contacts which connect this circuit board to upper circuit board 1305 (represented in FIG. 17), via FPC cables (not shown). Other example embodiments use other methods, such as, but not limited to, ZIF connectors, individual wires, and ribbon cables. 1608 is the microcontroller (MCU), and 1609 is the microcontroller's clock source. 1610 is the programmer for microcontroller 1608, and 1611 is the button used to enter programming mode in case of a non-responsive MCU, via 1610. Other example embodiments that do not require on-board programming do not require circuit blocks 1610 and 1611. 1612 is the current source and LED switching block, further described in FIG. 22.

FIGS. 17A and 17B show a front and back view of an example embodiment of an example layout for the upper melanin bias reducing pulse oximeter circuit board, also shown as 1305. 1701 is an example embodiment of a PCB which includes the traces and pads for the components. 1702 is the UI screen, further described in FIG. 22 shown mounted on 1701 visible through 1304, also 1006. 1703 is the user button mounted on 1701 and actuated, via 1302, also 1005. As mentioned above other example embodiments utilize other UI methods and therefore do not include UI screen 1702 or user button 1703. 1704 are the contacts which connect this circuit board to lower circuit board 1312, via FPC cables (not shown). Other example embodiments use other connecting methods, such as, but not limited to, ZIF connectors, individual wires, and ribbon cables. 1705 is the screen connection block which uses an FPC cable to connect screen 1702 to the other side of board 1701, via a cut-out in board 1701, to allow screen 1702 to mount flush. Other example embodiments use other connection methods for 1705, such as, but not limited to, SMD pins directly under the screen, ZIF, or BGA pins. 1706 shows the detector block which includes the analog photodiode, also 1405, further described in FIG. 22. 1707 is the analog voltage regulation block which powers the analog circuitry in blocks 1706 and 1708 to prevent high frequency noise from the digital circuits from interfering with the pulse oximetry readings. 1708 is the transimpedance amplifier, filtering, amplification, DC offset, and buffering block of the analog circuit. In other example embodiments, blocks 1706-1708 are replaced with other types of detection methods, such as, but not limited to, a photodiode with software filtering and/or a digital detector with software filtering.

Other example embodiments of FIGS. 16 and 17 use other integrated circuits, circuit board shapes, silkscreens, vias, circuit board layouts, and circuit blocks in order to achieve the same effect as the example embodiments explained in FIGS. 16 and 17, in accordance with the invention. The example embodiments shown in FIGS. 16 and 17 show double sided two-layer circuit boards. Other example embodiments of FIGS. 16 and 17 use other circuit layering combinations, such as, but not limited to, single sided, single sided two-layer, single sided multi-layer, and/or double-sided multi-layer.

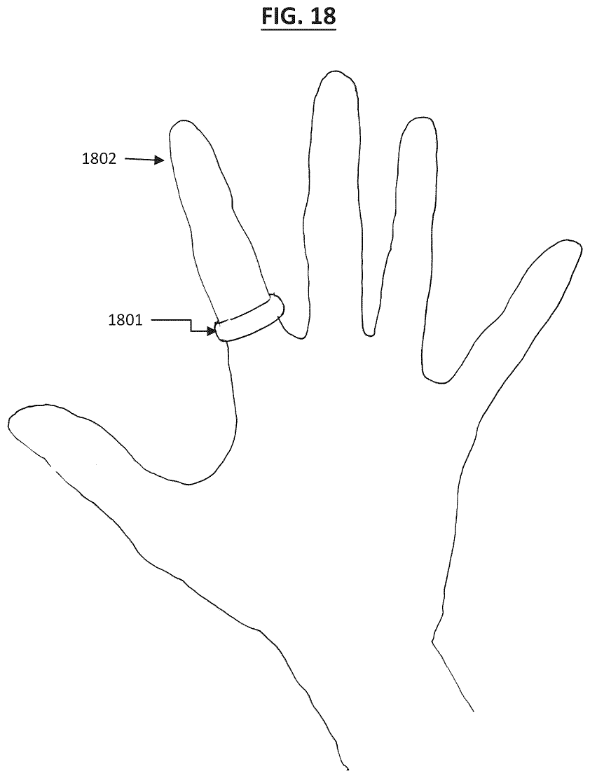

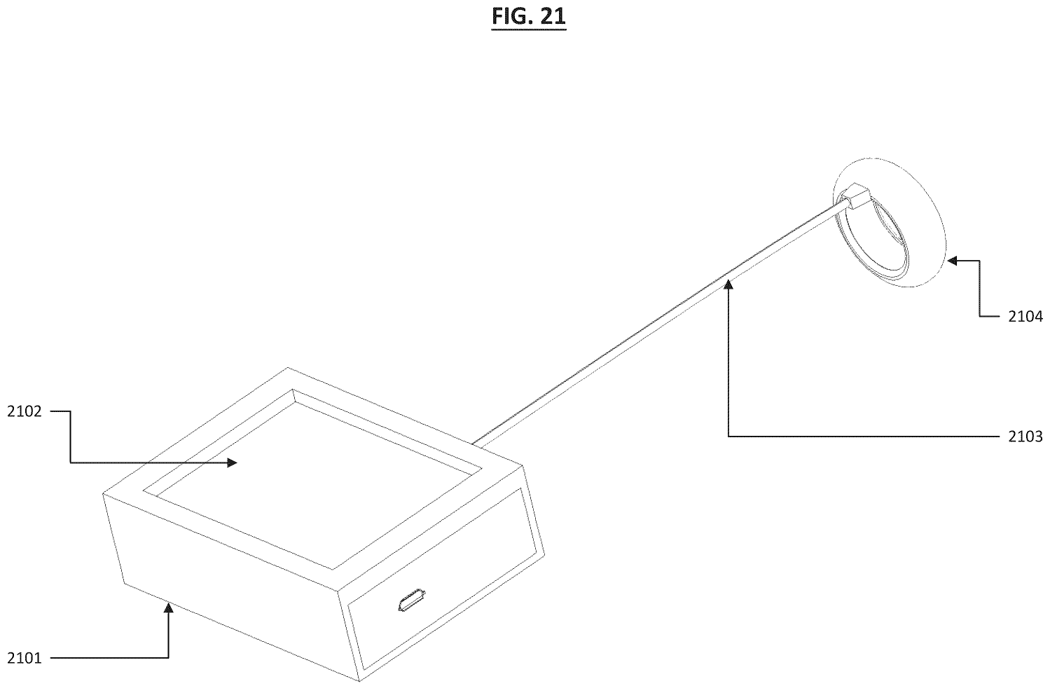

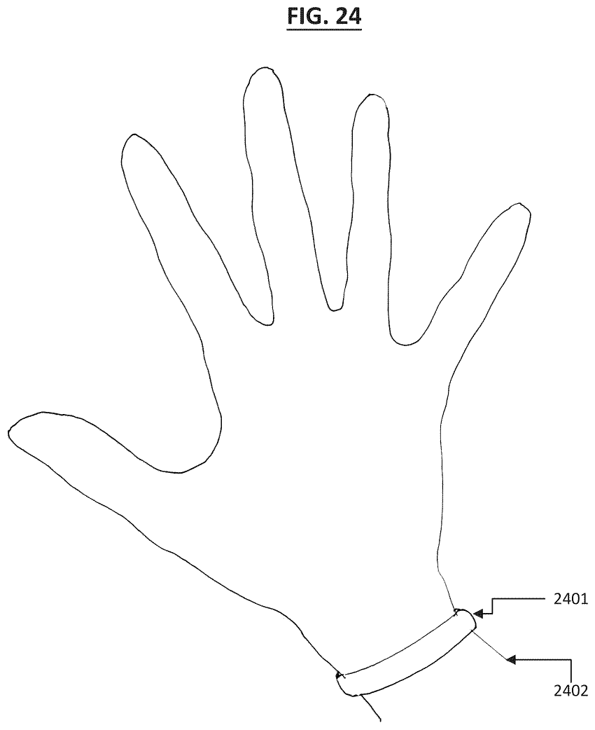

FIG. 18 shows a diagram of an example use of an example embodiment of a ring design of a melanin bias reducing pulse oximeter. 1801 shows the ring including the melanin bias reducing pulse oximeter, or in some example embodiments only the emitting and detector portions. 1802 is the user/subject/patient finger with ring 1801 on the finger as shown. Ring 1801 is wired, as shown in FIG. 21, or in other example embodiments is wireless connecting to the patient monitoring system or other UI. Other example embodiments of 1801 include, but are not limited to rings with reflective optical technology that go around a patient's arm or leg for patients who are amputees and/or rings used for infant monitoring that are wrist or ankle mounted, as further described as a bracelet or wearable flexible band in FIG. 24. Further, other embodiments of a bracelet or wearable flexible band design are used for patients who are unable to wear traditional pulse oximeters for reasons such as for example, but not limited to, anatomy, age, mental disability, sensitivity issues, and/or ADD.

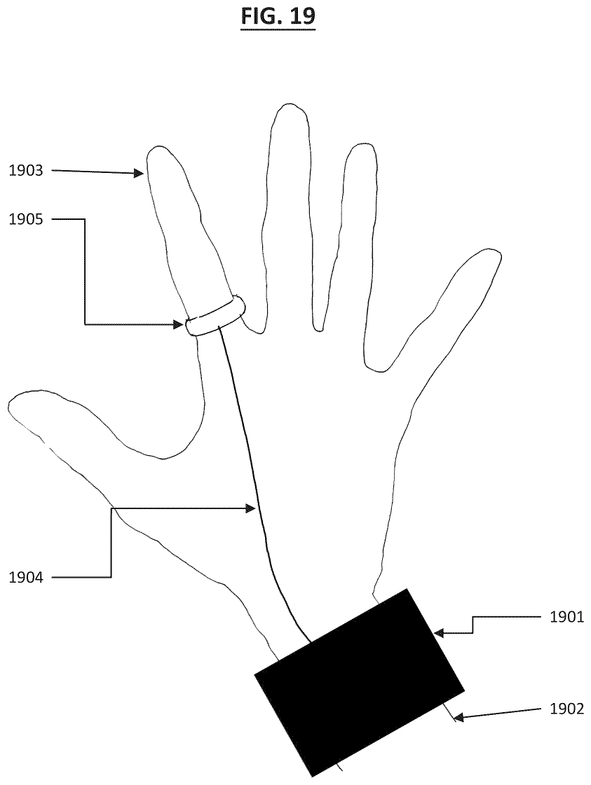

FIG. 19 shows an example usage of an example embodiment of a ring design of a melanin bias reducing pulse oximeter, including an example wrist mount user interface. 1901 is an example wrist mount interface, also shown as 2101 displaying the information shown in FIG. 12. Other example embodiments use interfaces, such as, but not limited to, interfaces mounted directly on ring 1905, bedside patient monitoring, portable patient monitoring, smart device interfaces, standalone handheld devices, and/or patient monitoring systems. These interfaces are connected to ring 1905, via cable 1904. Other example embodiments use other data transfer methods, such as, but not limited to, existing patient monitoring connections, magnetic contact communication, and/or wireless methods, such as, but not limited to, near field communication, WI-FI, BLUETOOTH, and/or proprietary communication protocols. 1902 is the arm/wrist that the user interface 1901 is mounted to. 1903 is the user finger, also 1802, that ring 1905 is placed on. Other example embodiments of FIG. 19 use other fingers than the ones shown (1903) for the placement of ring 1905. Further, other example embodiments of FIG. 19 use wrists, arms, or legs as mentioned previously for use in infant, toddler, and amputee monitoring or patients who are unable to wear traditional pulse oximeters for reasons such as for example, but not limited to, anatomy, age, mental disability, sensitivity issues, and/or ADD.

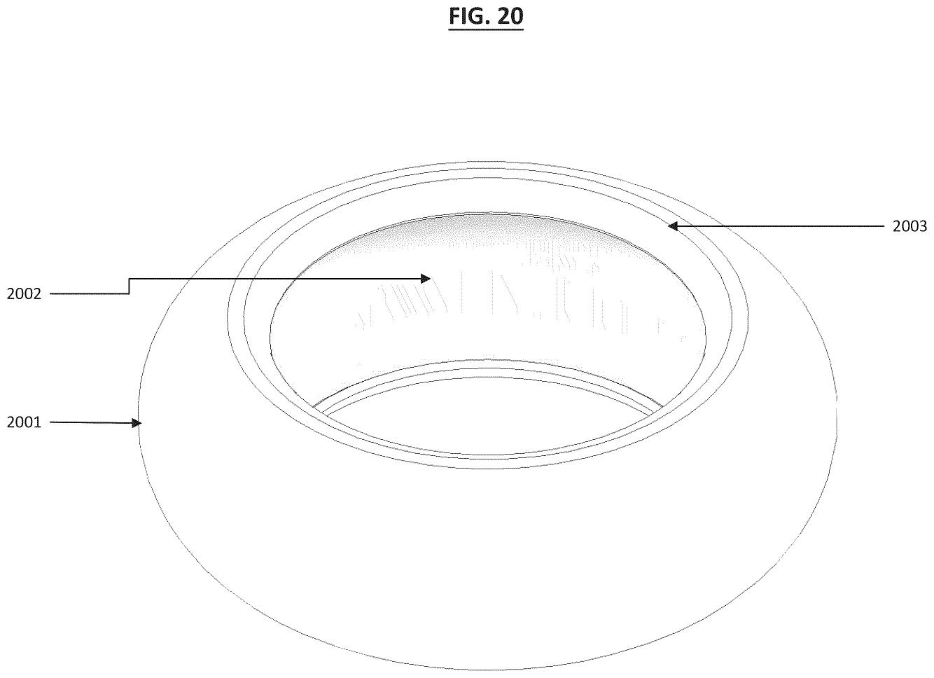

FIG. 20 shows a diagram of an example design of a flexible ring embodiment of a melanin bias reducing pulse oximeter. 2001 is the ring, also 1801, 1905, and 2104, which is made out of flexible rubber (TPU). Other example embodiments of the ring use other materials, such as, but not limited to, elastic, EVA foam, VELCRO strapping, medical grade rubbers, foams, and plastics, flexible breathable TEGADERM film, and/or other types of flexible rubber or plastic. Other example embodiments of the ring use medical grade material coated, treated, and/or designed with antimicrobial surfaces and/or chemicals. 2002 is the inner chamber of the ring that holds the flexible PCB including the optics, such as, for example, the emitter and detector mentioned as 1602 and 1706 covered by a protective casing made out of the same material as ring 2001. Other example embodiments use flexible circuit boards including, for example, a detector and emitter along with necessary filters and emitter control hardware. Further other example embodiments also include the microcontrollers, power sources, and wireless technology. 2003 is the lip that helps hold the flexible circuit board in place, as well as provides a smooth edge for the user's appendage to slip through. An example embodiment of ring 2001, as well as its internal components, is designed to be waterproof with some example embodiments, including intrinsic sealing, so that a patient can wear the pulse oximeter continuously, including wet locations such as the shower or bathroom.

FIG. 21 shows an example design of a flexible ring embodiment of a melanin bias reducing pulse oximeter with a wired user interface. Control box 2101 (also 1901) houses the control hardware shown in FIG. 13, including user interface screen 2102, also described in FIG. 12. 2103 is the connecting cable, also described in FIG. 19 as 1904. 2104 shows the ring, also described in FIG. 20, also 1905 and 1801. 2104 and 2101 as shown have a strain relief for cable 2103. As previously discussed, other embodiments of the ring design take on other forms, use other user interfaces, and other communication methods than those shown in FIG. 21. An example embodiment of 2101, as well as its internal components, along with ring 2104 (as described in FIG. 20) is designed to be waterproof with some example embodiments including intrinsic sealing.

FIG. 22 shows a broad overview of a general hardware signal flow for an example embodiment of a melanin bias reducing pulse oximeter system and is an example of how components on the circuit boards shown in FIGS. 16 and 17 interact with each other. User 2201 interacts with the hardware, via user button 2202, also 1703, programmer button 2204, also 1611, and LCD screen 2203, also 1702. Button presses from user button 2202 are sent to the power management logic 2213, also 1606, which controls power up, steady state, and power off of the circuit. Program button 2204 communicates with programmer bootloader IC 2205, in order to update firmware on MCU 2207, using data from the USB port 2206 (also 1603). Some example commercial embodiments, which do not require the user to have direct firmware access, do not include the computer data portion of USB port 2206, programmer bootloader 2205, also 1610, programming button 2204, also 1611, and independent power management logic (part of 2213).

Program button 2204 is only required to be pressed by user 2201 when the USB part of programmer bootloader 2205 is unresponsive. Independent latching circuitry in power management logic 2213 is required because microcontroller 2207, also 1608, has a delayed boot while waiting for programming bootloader 2205 to release it from program waiting mode 2301. Other example embodiments of the hardware use other programming and firmware update methods than those shown, such as, but not limited to, ICSP, JTAG, SWD, UART, SPI, parallel, and/or wireless update methods, some of which do not require dedicated programmer bootloader IC 2205.

MCU 2207 in the example embodiment shown is an NXP Kinetis ARM Cortex M4 running the Teensy Arduino platform with a clock speed of 72 MHz generated by scaling 16 MHz program clock 2208, also 1609. The Kinetis MCU 2207 is chosen for the example embodiment shown due to the on-board 12-bit ADC and DAC, so that the circuit would require minimum external components. Other example embodiments use other MCU ICs instead of the Kinetis MCU 2207, such as, but not limited to, Nordic ICs, other Arduino compatible ICs, and other microcontrollers with suitable peripherals known to those in the field.

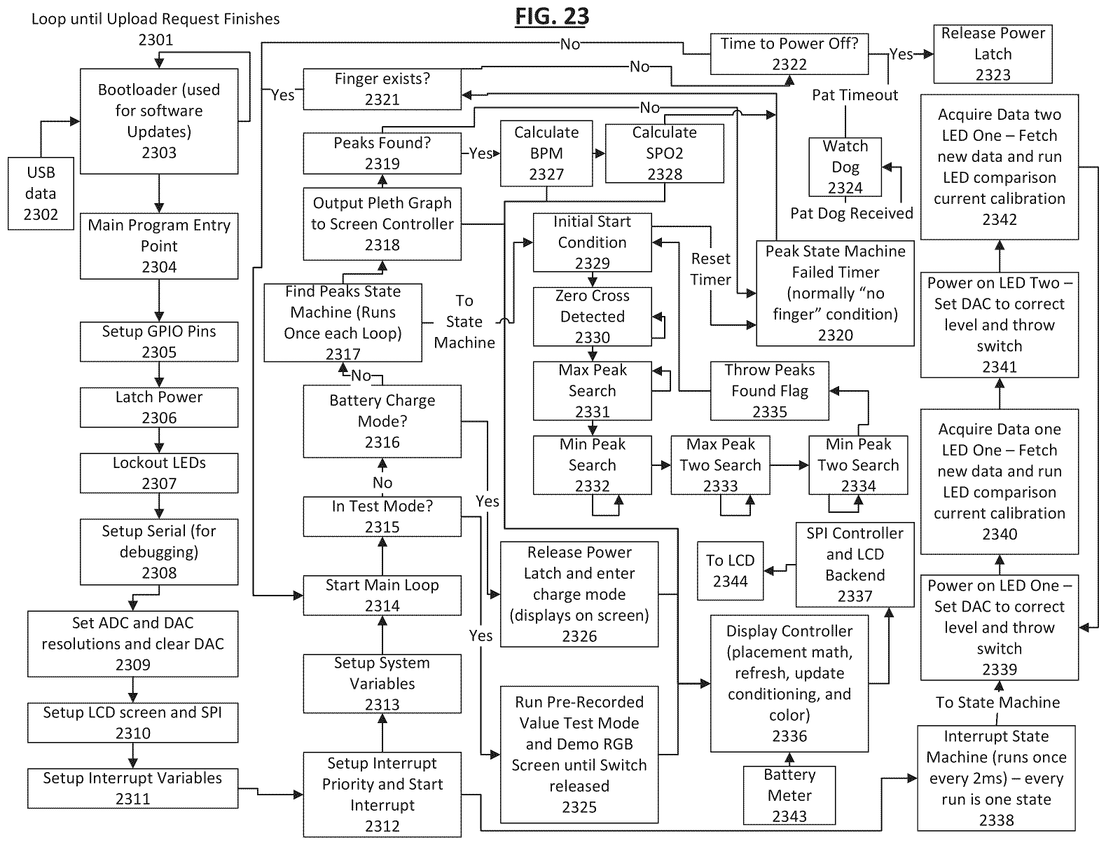

MCU 2207 runs the software described in FIG. 23, which controls the hardware peripherals and inputs and outputs. DAC output 2233 on MCU 2207 controls the current driver logic 2217 (also part of 1612), which is used to control LED 1(2219) and LED 2(2220), also 1602, through a 2:1 Mux (multiplexer) 2218, also part of 1612. Other example embodiments use other methods to control 2219 and 2220, such as, but not limited to, addressable LEDs, filtered PWM, and/or constant current driver ICs. Mux 2218 uses the second half of the IC to create an LED lock-out through IC loop 2236, controlled along with LED switching, via 2234, via 2207, in order to prevent LED damage during DAC 2233 settling during initial boot. LED 2219 and LED 2220 transmit light through human subject/user/patient finger 2221, also 1101 and 1802, to be received by photodiode 2222, also 1706.

In the example embodiment shown, LED 1 2219 is a about 768 nm LED, and LED 2 2220 is a about 960 nm LED. These wavelengths were chosen based on test bench, see FIG. 1, data classifying these LED wavelengths as the combination that has the best transmissive effect in melanin testing for reducing the melanin bias. Other example embodiments use other wavelengths, such as, but not limited to, wavelengths currently on the market, about 640 nm and about 960 nm and/or other IR wavelength combinations. Wavelength combinations currently on the market do not solve the melanin bias issue, but require little additional approval from federal regulators to use with some of the example embodiments, such as, but not limited to, the ring embodiment explained in this document. 2219 and 2220 in the example embodiment shown are separate SMD components. In other example embodiments, in order to save space, both LEDs are combined into a single package.

In further example embodiments in which LEDs are combined into a single package, they are electrically designed to operate each emitter in reverse polarity to each other. This design further reduces the component count. Further, in other example embodiments, instead of LED (2219-2220) light passing through finger 2221 to enter photodiode 2222, the light instead enters finger 2221 and reflects back into photodiode 2222. Light entering photodiode 2222 produces a small current that is converted into a usable voltage using transimpedance amplifier 2224, also part of 1708. Other example embodiments use photodiodes with built in transimpedance amplifiers. Other example embodiments use lasers and other light emission methods instead of LEDs 2219 and 2220, while other example embodiments use phototransistors and other light detection methods instead of photodiode 2222.

Furthermore, other example embodiments use digital light detection methods, instead of photodiodes, making the remaining portion of the analog circuit unnecessary as this filtering is done in software. Filter channel switch 2227, controlled by LED switch signal 2234, pipes the intensity voltage data returned from each LED into its own filter, 2228 and 2229, in order to ensure each returned wave form is tracked separately by MCU 2207. Filters 2228 and 2229 are modified off NPX's reference sheet for filter design. The output of filters 2228 and 2229, also part of 1708, are amplified, via 2230 and 2231 before being sent to ADC 2232 on MCU 2207. Filters 2228 and 2229 include, for example, analog filters including, but not limited to, high pass, low pass, notch, low cut, and high cut filters. The analog circuit components are powered, via 2226, also 1707, controlled by power management logic 2213.

Transimpedance amplifier 2224 is buffered, via 2223 before having its signal independently sent to MCU 2207, via ADC 2232, for processing. Gain switch 2225 is controlled by MCU 2207, via GPIO LED switch 2234. Gain switch 2225 is responsible for the leveling of the DC waveform components, as well as intensity returns of both LEDs. Half rail power buffer 2237, also part of 1708, is responsible for providing power to the analog filter and amplification stages to simulate a negative reference point, in order to make the waves reproducible by a single supply voltage source. In other example embodiments, the transimpedance output is sent directly to MCU 2207 and software filtering replaces the remaining portions of the analog circuit. Further, in other example embodiments in which digital detector methods are used, software filters are used in a similar fashion. Amplifiers 2230, 2231, and transimpedance amplifier 2224 in example embodiments shown, are based off of high precision op-amps.

Other example embodiments use other types of amplifiers, including, but not limited to, other types of op-amps, instrumentation amplifiers, differential amplifiers, and purpose-built ICs. Embodiments which use other types of amplifiers require additional software modifications to the software described in FIG. 23. Power regulation 2214 and 2215, also 1605, are sent through power selector logic 2216, in order to power the digital side of the circuit. 2214 is used when running off of Lipo battery 2209. 2215 is used when running off of USB power 2206, also 1603. Lipo battery 2209 is controlled and charged, via charging logic and management 2211, also 1604.

During charging, logic 2211 receives power from USB 2206. Logic 2211 works with temperature sensor 2210, placed on battery 2209, in order to ensure safe and efficient charging. In the example embodiment shown, purpose-built ICs are used for charging logic and management 2211. Other example embodiments use other forms for charging logic and management, such as, but not limited to, MCU driven charging, constant current charging, USB diode-controlled charging, and/or removable batteries with external chargers. Lipo battery 2209 is used due to its efficiency and energy density. Other example embodiments use other types of batteries, such as, alkaline, nickel cadmium, lead acid, AGM, gel, lithium ion, lithium phosphate, solid state, sodium composition, ceramic, kinetic, and/or removable rechargeable batteries.

Battery meter logic 2212 reports battery usage information to MCU 2207. In the example embodiment shown, 2212 is based off a voltage divider design, however, other example embodiments use other battery meter methods, such as, but not limited to, state of charge ICs, fuel gauge ICs, and/or Coulomb counters. 2235 is the SPI bus on MCU 2207 that interfaces with LCD screen 2203. Other example embodiments use other data communication methods to communicate with their visual outputs, such as, but not limited to, I²C, 1-wire, UART, and parallel. LCD screen 2203 in the example embodiment shown is an RGB LCD screen. An RGB screen is used, in order to use colors to represent different oxygen level ranges to increase clear interpretation and ease of use for a layperson. Other example embodiments that do not require this enhanced feature may use monochrome screens, such as, but not limited to, E-ink, single color OLED, LED arrays, and gray scale LCDs.

Further, other example embodiments use other dimensions and configurations of screens, as well as other types of screens, such as, but not limited to, OLED, smart device interfaces, and/or computer interfaces. Other example embodiments that require wireless technology use, for example, external wireless transceivers and transceiver ICs with data streams connected to MCU 2207, while other example embodiments use other wireless technologies, such as, for example, SoCs. FIG. 22 represents an example method of connecting components and their signal paths, and those experienced in the field will recognize that there are other methods, signal paths, and ICs to produce the same result in accordance with the invention.

Other example embodiments have audio and/or voice synthesizers as part of their UI to give an auditory message regarding the readings displayed on screen to increase ease of use and make the device visual impairment friendly. Further, other embodiments, for example ring and flexible bracelet embodiments, include additional hardware not shown in FIG. 22, which includes for example, but not limited to, ECG, temperature, CO2, blood sugar and other blood gas information sensors. Additionally, hospital grade devices require shielding both against outside interference and to prevent unwanted interference with other medical devices or implants. Example embodiments utilize many methods of shielding, including, but not limited to, MRI hardening, electromagnetic interference shielding, and/or radio frequency interference shielding. Examples of this shielding include a grounded shield placed over the electronic components, conductive sprayed coating inside the case of the device, and/or braided shielding around openings in the device case.

FIG. 23 shows a general overview of a software signal flow that runs, for example, on MCU 2207 of one example embodiment of a melanin bias reducing pulse oximeter system. The entry point of the program starts at 2301, which loops until the pending upload request 2301 from USB data 2302 finishes, or until bootloader 2303 releases the software update stage. 2301 and 2203 are controlled by the bootloader IC 2205. Other example embodiments use other data streams for program data other than USB data 2302, such as, but not limited to, ICSP, JTAG, SWD, UART, SPI, and/or wireless update methods. Further, other example embodiments that do not give the user direct access to perform firmware/software updates, do not require blocks 2301-2303, and these embodiments start at block 2304. Once bootloader 2303 exits, main program entry point 2304 is entered. Main program entry point 2304 begins the set-up process, which includes setting up GPIO pins 2305, latching power control 2306, LED lock out 2307, setting up serial debugging 2308 (not performed in many commercially released embodiments), setting ADC and DAC resolutions, as well as clearing DAC data 2309, and setting up LCD screen and SPI bus 2310. This set up continues by setting up interrupt variables 2311, setting up interrupt priority, and starting interrupt 2312, which has a starting entry point of 2338, setting up system variables 2313, and entering main loop 2314. Further, other example embodiments use another order for initial setup steps, in order to produce the same resultant effect, in accordance with the invention.

Main loop 2314 is a loop in which the program runs all remaining functions from and only temporarily exits to run interrupts, or permanently exits when the release of power latch block 2323 runs at program exit. Main loop 2314 first checks if the system is in test mode 2315, which is used for verifying that all components are functioning, as well as demonstrating the user interface. If the system is in test mode, block 2325 runs, which outputs a pre-recorded set of intensity values to the BPM and SPO2 control blocks, which outputs their information to the display controller 2336. If the system is not in test mode, the system checks if battery charge mode 2316 should be entered. If so, charge mode 2326 is entered and the power latch is released, so that the system will automatically power off when the charging source is removed. Charge mode 2326 displays the charging icons on the screen, via the display controller 2336. If the system should not enter charging mode, the peak state machine runs, 2317. The state machine runs once per loop. The state machine has the following states, which are used during the peak found block, 2319. The states are, initial condition 2329, zero cross detection 2330, which loops until zero cross is found, max peak search 2331, which runs until a maximum peak is found, min peak search 2332, which runs until a min peak is found, max peak 2 search 2333, which runs until the second max peak is found, and min peak 2 search 2334, which runs until the second minimum peak is found. By finding two min and max peaks, the system is able to calculate beats per minute in the BPM calculation block 2327 by calculating the time between peaks. Once all peaks are found, the peaks found flag is thrown in 2335, which is used by 2319.

During the initial start process 2329, the timer for detecting if the state machine failed in block 2320 is reset. A state machine failure, being triggered from timer 2320, normally occurs if the user's/subject's/patient's finger is removed from the pulse oximeter, which is checked in block 2321. If a finger still exists in block 2321, the main loop starts over in 2314. If the finger does not exist in block 2321, the timer for power off in 2322 is checked. If it is not time to power off, again the loop starts over in 2314, otherwise, the power latch is released in 2323, and the program exits when the device shuts off. After the state machine runs in 2317, the pleth graph is generated and output to the screen controller 2336 in 2318. The pleth graph is generated by taking raw intensity data and using a maximization algorithm to dynamically stretch the data to appropriately fit the screen without distorting it. After outputting the pleth graph, it is checked whether peaks have been found in 2319. If no peaks are found, the peak state machine failed timer is queried in 2320, otherwise, BPM is calculated in 2327 and SPO₂ is calculated in 2328, both of which output their data to display controller 2336, before verifying if the finger still exists in 2321.

Battery meter 2343 also outputs its data to display controller 2336 as new battery states are delivered. Display controller 2336 is responsible for refresh commands, update conditioning to ensure reduced flickering during updates, color management, text scaling, and screen placement math, in order to create a bitmap, which is sent to SPI controller software and LCD back-end software 2337. 2337 is responsible for sending appropriate SPI commands and data streams to control LCD 2344, also 2203. Watch dog timer 2324 runs in the background and is “patted” during each iteration of the loop. If the watch dog timer 2324 has a “pat” time-out, in which the loop has failed to “pat” the watch dog before the timer runs out, it will force a release of the power latch 2323 to shut off the device and end any hung software. Interrupt entry point 2338 is the starting point for the interrupt 2312, which runs once every 2 ms and temporarily halts the main loop while it runs. The interrupt function itself is a state machine where every state is one run of the interrupt.

Other example embodiments use other interrupt run times depending on processor load and required capture times. Further, other example embodiments use a multi-core MCU to independently run the state machine and other functions on separate cores/threads. The first state of the interrupt state machine 2339, which powers on the first LED (2219) by setting the correct DAC level (2233) and throwing the switch (also Mux 2218). The second state 2340 acquires data from the first LED (2219) by running LED comparison calibrations used to level both LED DC offsets and then fetching and recording the data. The LED controller (also Mux 2218) is also put into lock-out mode at the end of this state, in order to prevent damage to the LED during the following state. The next state 2341 powers on the next LED (2220) by setting the correct DAC level (2233) and throwing the switch (also Mux 2218). The final state 2342 acquires data from the second LED (2220) by running LED comparison calibrations used to level both LED DC offsets and then fetching and recording the data. The LED controller (also Mux 2218) is also put into lock-out mode at the end of this state in order to prevent damage to the LED during the following state. The state machine repeats and goes back to state 2339 on the next interrupt iteration.

Other example embodiments that require wireless transmission have wireless transmission blocks connected to the display controller 2336, and/or send pre-display controller data structures wirelessly for display on the UI device. Further, other example embodiments use other software designs, block ordering, state machines, and software signal flows, in order to produce the same resultant effect, in accordance with the invention. Example embodiments that use wireless transmissions have wireless control blocks in other locations in the software flow. These wireless example embodiments include, but are not limited to, the finger clip and ring embodiments further described in this document. An example of the wireless embodiment implements filtering previously described as analog hardware using digital filtering and averaging methods such as Infinite Impulse Response Filtering, Window-Sinc filtering, Finite Impulse Response Filtering, Moving Average Filtering, Butterworth Filtering, Asymmetrical Kalman Filtering, Kalman Filtering, Wavelet Denoise Filtering, Savitzky-Golay Filtering, Root Mean Square Averaging, and Weighted Moving Averaging. Further other example embodiments utilize a Window-Sinc Filter with a Spectral Inversion for filtering frequencies undesirable for pulse oximetry. In some example embodiments Fast Fourier Transform and Discrete Cosine Transform is used to assist in the filtering process.

The software is responsible for controlling the LEDs (emitters), as well as the detection hardware. In one example embodiment the pulse oximeter software begins the SpO₂ determination by increasing the intensity of each emitter separately to determine the peak point prior to parasitic scattering due to the intensity level. That is, one or more processors control a first emitter to increase the intensity of the first emitter and monitor an output of a light detector on which the light generated by the first emitter impinges until a peak point is detected where the peak point occurs before parasitic scattering is detected. The one or more processors repeat the process with each additional emitter. The peak point represents the maximum emitter intensity to overcome absorption while still preventing parasitic scattering.

In other exemplary embodiments, the one or more processors compare the emitters' signals to level (or make equal) the returned DC offset. For example, both emitters DC offsets are leveled. The intensity to level at determined by the maximum intensity found above when finding the peak for parasitic scattering. The lesser of both emitters' peaks is used here. The first emitter is turned on and the detector output is captured while the first emitter is turned on. Then the first emitter is turned off and the second emitter is turned on. The output of the detector is captured while the second emitter is turned on. The second emitter is turned off and the first emitter is turned on again. The process of both emitters flip flopping and the output of the detector being captured repeats until the system is turned off or a finger is removed from the device. The data captured from the output of the detector is processed and filtered separately (e.g., each emitter and corresponding output is considered separately) with the DC component removed to generate pleth graphs (with the AC data). The pleth graph is displayed and the peaks of the graph are used to identify a heart rate. The AC components from both graphs are used to determine the SPO₂ ratio and a lookup table is used to relate this to SpO₂.

In embodiments that utilize digital filtering, the analog filtering described in the hardware section of this document is not used. These embodiments drastically reduce component requirements and provide for easy calibration in software. Similar filtering to that described in hardware are implemented using software techniques that reproduce analog filters, including but not limited to, high pass, low pass, notch, low cut, and high cut filters. Some example embodiments also perform additional filtering, averaging, and signal processing. The state machines as described previously run to record data from both emitters as detected by the detector. As previously described the DC offset is removed and a single emitter's data is used for the displayed pleth graph and the heart rate calculations (based on the pleth peaks). Other example embodiments utilize an average of the emitters' data for the displayed pleth graph and the heart rate calculations (based on the pleth peaks). The pleth graphs without the DC offset are then compared to determine the difference in the AC portions representing the oxyhemoglobin and deoxyhemoglobin found in the subject's blood. This difference is used to calculate the R value or ratio previously described to determine the SpO₂ based on correlating the R value with a calibration lookup table. Further example embodiments utilize other methods of pulse oximetry, as described, while still utilizing the melanin bias reducing optical configurations described in this document. One of these example embodiments utilizes one melanin bias reducing emitter with a BQJ detector with four channel outputs to calculate SpO₂ utilizing the pulse oximetry principles and software described, but instead utilizing the four channels to determine the spectral differences in oxyhemoglobin and deoxyhemoglobin with an individual emitter source to determine SpO₂. Further example embodiments utilize temperature sensing near or within the emitters to monitor and adjust for wavelength drift due to the temperature of the emitter. Example embodiments that include wireless transmission capabilities also include software for assigning an ID (or device/patient identifier) to the pulse oximeter for use in monitoring systems. The ID is used to identify which patient the pulse oximeter is monitoring when connected to a monitoring system, which routes the data to the correct patient monitoring display within the patient monitoring network. This ID functionality is performed in a similar manner to the ID functionality described further in the heart telemetry section of this document.

FIG. 24 shows a diagram of an example use of an example embodiment of a bracelet design or wearable flexible band of a melanin bias reducing pulse oximeter, similar to the ring shown in FIG. 18. 2402 shows the bracelet including the pulse oximeter, or in other example embodiments only the emitting and detector portions. 2401 is the user/subject/patient arm or wrist, with the bracelet 2402, placed on it as shown. Bracelet 2402 is wired in a similar method to the ring embodiment, as shown in FIG. 21, or is wireless connected to the patient monitoring system or other UI as previously described. Other example embodiments of 2402 include, but are not limited to, bracelets with reflective optical technology that go around a patient's arm or leg for patients who are amputees and/or bracelets used for infant and toddler monitoring that are ankle/wrist worn. Further, other embodiments of a bracelet or wearable flexible band design are used for patients who are unable to wear traditional pulse oximeters for reasons such as for example, but not limited to, anatomy, age, mental disability, sensitivity issues, and/or ADD. Bracelet 2402 is designed utilizing similar materials and similar fashion, as described in FIG. 20 for the ring embodiment. This design allows for flexibility in the size of the appendage the bracelet is placed on.

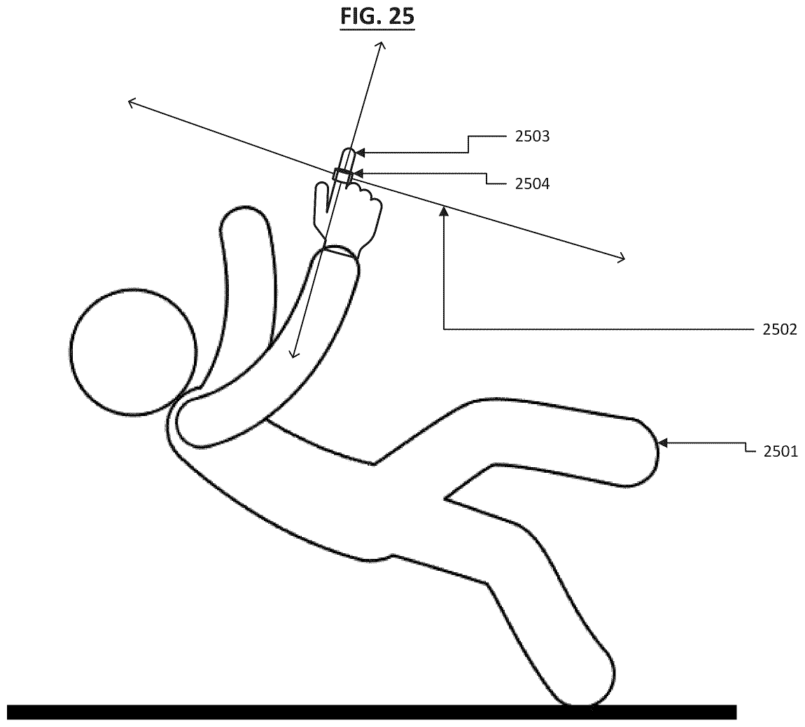

FIG. 25 is a diagram showing fall detection and bed alarm integration for a melanin bias reducing pulse oximeter system. 2501 demonstrates a user in the process of falling. 2502 shows examples of the force vectors being registered by the pulse oximeter, indicating that user 2501 is falling. 2503 represents the appendage the pulse oximeter is placed on. 2504 represents the pulse oximeter on the appendage. All embodiments of the pulse oximeter system are capable of implementing this feature utilizing additional hardware in FIG. 22, such as, for example, but not limited to, a gyro sensor for measuring angular rate, accelerometers for measuring acceleration and velocity, altitude sensors for measuring height, angular velocity sensors for measuring angular velocity (some manufacturers consider these gyro sensors or gyroscopes), magnetometers for measuring magnetic field and providing a digital compass, and/or single or multi axis inertia sensors often referred to as an IMU or inertial measurement unit. Further, other embodiments implement a sensor fusion algorithm which combines the data from multiple sensors previously mentioned to provide more accurate sets of measurement data. The ring and bracelet embodiments are the most practical and useful embodiments when used in a wireless system to include this fall detection and bed alarm capability. An example usage of this technology is in hospital and nursing home settings for patient monitoring that is comfortable for the patient and easy to implement. The technology described in FIG. 25 is also used in other example embodiments for comfortable and easy multi-location bed, chair, and bathroom exit/standing alarms functioning similarly to the current more cumbersome bed and chair alarm systems.

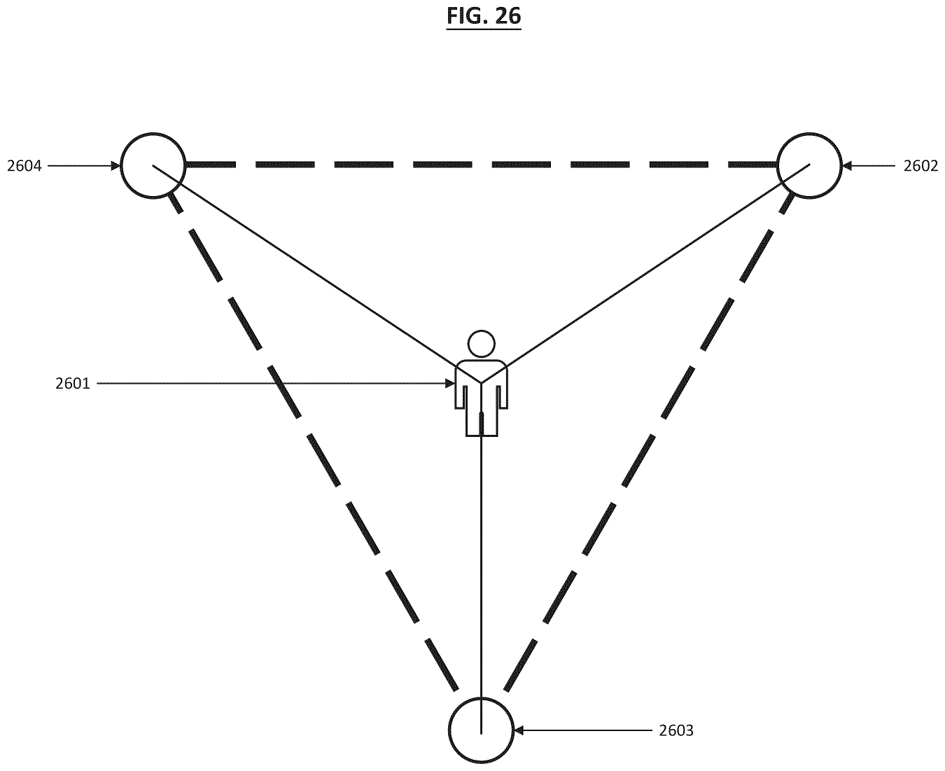

FIG. 26 shows a diagram of an example location detection method for the melanin bias reducing pulse oximeter system. All embodiments of the pulse oximeter system are capable of implementing this feature utilizing additional hardware in FIG. 22, such as, for example, but not limited to, GPS, WI-FI triangulation, cell tower triangulation, mesh networks, RFID/NFC, or similar type door and hallway monitor sensors, and/or proprietary radio system triangulation. The ring and bracelet embodiments are the most practical and useful embodiments when used in a wireless system to include this location detection capability. An example usage of this technology is in hospital and nursing home settings for patient monitoring that is comfortable for the patient and easy to implement. 2601 represents the location of the user, and 2602-2604 represents the communication stations in which the triangulation is performed. Other example embodiments, use for example, but not limited to, GPS links to perform the tracking function and therefore do not require radio triangulation on the same plane as the user/subject/patient as shown in FIG. 26. Further, other example embodiments use this technology as a tracking device to assist family members in geo-fencing as well as retrieving location information for children, adults with disabilities, and the elderly while also providing monitoring of vitals in the same device.

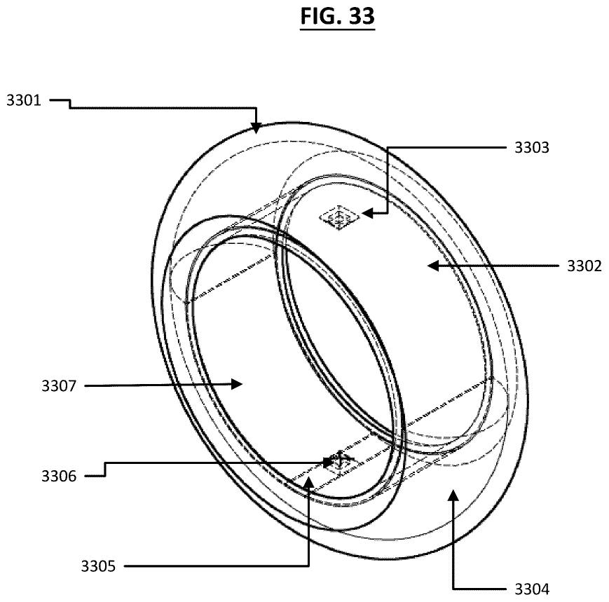

FIG. 33 shows a diagram of an example design of a flexible ring embodiment of a pulse oximeter including the internal components' arrangement to provide flexible adjustment. 3301 is the outer case of the ring, as described in FIG. 20. 3301 is made out of a flexible material in example embodiments that require a stretchable band for either a snug fit or to provide an adjustable ring for different size appendages. Flexible embodiments are made of materials which are described in FIG. 20, and include, but are not limited to flexible rubber, TPU, elastic, EVA foam, VELCRO strapping, medical grade rubbers, foams, and plastics, flexible breathable TEGADERM film, silicon, and/or other types of flexible rubbers or plastics known in the field. Other example embodiments of 3301 include rigid rings similar to a wedding band. In rigid embodiments, 3301 is made of plastics, silicones, metals, rigid rubbers, medical grade materials, and/or other band materials known in the field. As mentioned in FIG. 20 other example ring embodiments are coated, treated, and/or designed with antimicrobial surface, finishes, and/or chemicals.

FIG. 33 shows an example layout of the components found in the inner chamber of the ring previously described in 2002 and FIG. 22. FIG. 33 shows an example embodiment in which the battery 3304 and PCB 3303 each take up half of the ring. This design keeps in mind the flexible requirement of example ring embodiments by ensuring the battery does not surround the ring. Other example embodiments in which the ring is outer case rigid, battery 3304 and PCB 3302 take on different configurations. One example of these additional configurations is a battery 3304 that follows the circumference of the ring to provide additional battery capacity in a non-rigid form. Additional configurations of PCB 3302 and battery 3304 are possible and will be obvious to those in the field. PCB 3302 includes the majority of the on-board electronic components required for the ring to function.

One of these components shown is the detector 3303. In the embodiment shown, detector 3303 is an analog photodiode which outputs a current signal based on the light impinged on the detector from the emitter 3306. Other example embodiments use a detector 3303 that includes a transimpedance amplifier on one integrated circuit to reduce the component count required and to reduce energy consumption. Further, additional example embodiments use a detector 3303 that includes all required circuits to produce a digital signal, therefore eliminating the need for an ADC (analog to digital converter), and therefore reduces noise interference between the detector and the microcontroller (MCU or processor). PCB 3302, also includes all hardware required for the pulse oximeter to function.

One example embodiment of the PCB 3302 includes one or more MCU (microcontroller or processor) which runs the main software program, the power management hardware, the DAC (digital to analog converter) for the emitter constant current controller, the ADC (analog to digital converter), the transimpedance amplifier, filtering, gain control, battery charging controller, battery gauge hardware, and user interference connection. Example embodiments that include wireless capabilities also include wireless components on PCB 3302. Example embodiments of charging methods include, but are not limited to, magnetic charging, inductive charging, micro USB, mini USB, USB-C, waterproof micro USB, waterproof mini USB, waterproof USB-C, QI wireless charging, and charging contacts on a charging cradle. Example embodiments that use inductive or QI wireless charging include a wireless charging coil, not shown.

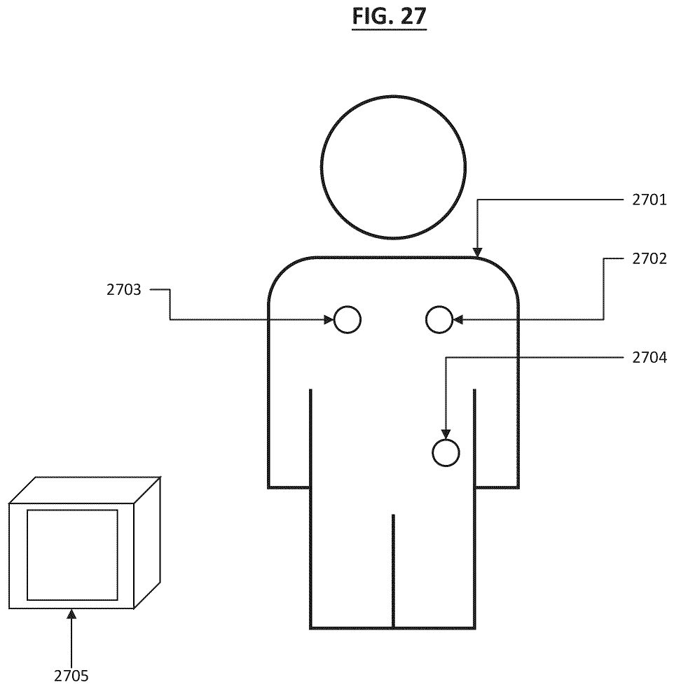

Further example embodiments that use charging methods with external contacts are not visualized in the example embodiment drawing. One example embodiment uses Nordic MCU nrf52832 or nrf52840 due to the onboard BLUETOOTH Low Energy (BLE) system on chip (SoC), as well as BLUETOOTH mesh, THREAD, ZIGBEE, 802.15.4, ANT and 2.4 GHz proprietary SoC stacks. Further other example embodiments use other MCUs with other wireless capabilities as further described in FIG. 22 and further expounded on in FIG. 27. Other example embodiments use a digital method for filtering and amplification to reduce component counts as described in described in FIG. 22 and FIG. 23. Further, other example embodiments use other component count reducing methods such as an MCU with a built-in ADC and/or DAC, as well as other LED control methods. Other example embodiments additionally use a software-based battery gauge and charging control method to further reduce component counts. One example embodiment uses wafer level components on flexible PCB 3302 in order to reduce the size of the required components. Other example embodiments switch the positions of emitter 3306 and detector 3303.

Further, other example embodiments have multiple emitters and detectors spaced around the ring to provide more options for light transmission through the appendage. This example multiple emitter and detector configuration is used to reduce issues that arise with poor contact of some emitters and detectors depending on the position of the ring. That is, with the multiple emitter and detector configuration adequate coverage can be achieved with less than all of the emitters and detectors being in contact with the appendage.

Additionally, other example embodiments use reflective technology for the detector and emitter. In these example embodiments, the second PCB section 3305 including the emitter 3306 is not necessary as the detector and emitter are placed near each other on the same PCB 3302. Further, other example embodiments include a self-contained photoplethysmography (PPG) sensor integrated circuit (IC) instead of detector 3303 and emitter 3306. An example melanin bias reducing PPG sensor is described further in this document. As previously mentioned 3306 represents the emitter mounted on a small flexible PCB 3305. PCB 3305 provides a substrate to solder emitter 3306 to, as well as providing a way to securely electrically connect to PCB 3302.

One example embodiment uses flexible ribbon cable to connect the two PCBs, while other example embodiments use small individual wires or twisted pairs to connect the two PCBs. Emitter 3306, represents at least two light sources as further described in this document. An example embodiment uses separate LEDs configured next to each other, while other example embodiments use a single LED body including separate emitters that can be controlled independently. Further example embodiments use other types and styles of light sources as described in this document. Additional example embodiments that use a single LED body with separate emitters do not use PCB 3305 as a mounting substrate and have a ribbon cable or individual wires or twisted pairs coming directly off the LED body to connect to PCB 3302. 3304 is the battery used to power the electronic components embedded in the ring. In the example embodiment shown 3304 occupies half the ring to provide the flexibility of the ring to stretch around the battery for different sized appendages.

Other example embodiments use other battery configurations to provide more or less flexibility/rigidity as required. The battery 3304 is described further in FIG. 22. However, in the ring configuration of the pulse oximeter the battery is designed to follow the curved contour of the ring shape as shown in the example embodiment. Many example embodiments use a standard LiPo battery shaped with a ring in mind. However, these batteries have a very low tolerance to flexing. Therefore other example embodiments use a solid-state battery or a battery/power source that is highly flexible. In the example embodiment shown battery 3304 is connected directly to PCB 3302 on one end, while the other end is not attached to provide the expandable function of the ring to fit multiple sized appendages. In other example embodiments the battery uses similar electrical connection methods as emitter 3306, such as, but not limited, to ribbon cables, wires, and/or twisted pairs. 3307, represents the flexible cover that separates the internal components from the appendage the ring is worn on. The cover has cutouts for detector 3303 and emitter 3306.

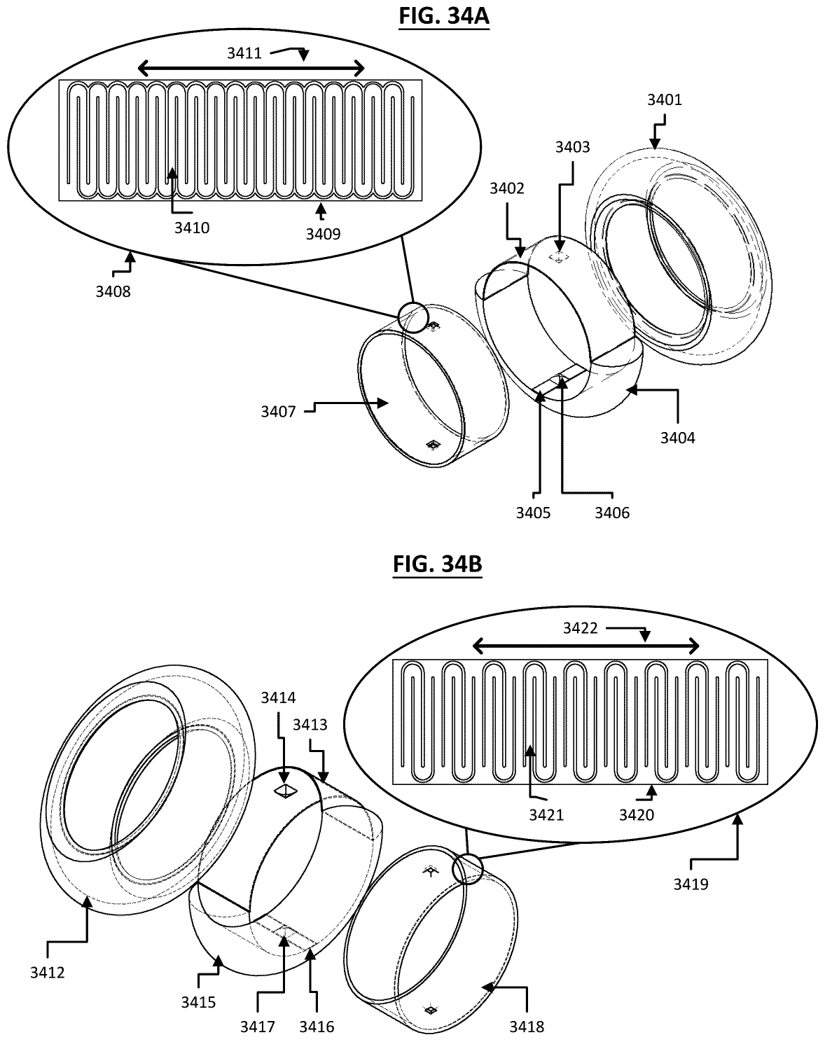

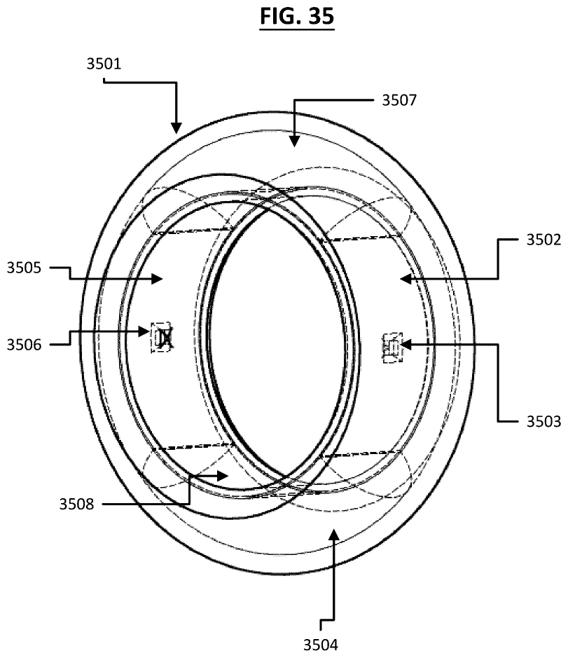

The detector and emitter are designed to stay in fixed positions while the ring expands or contracts around them with the other ring components shifting as necessary. This process is further described in FIG. 34. 3307 is designed using the same materials as described elsewhere in this document for 3301 and FIG. 20. All embodiments described in this section have the emitter 3306 and detector 3303 configured, as described elsewhere in this document, to include melanin bias reducing optical properties. Further, other example embodiments include a detector 3303 and emitter 3306, as known in the field, and are currently on the market. These example embodiments include optics that do not reduce melanin bias and are built using current technology and off the shelf parts, including, but not limited to, on the market PPG sensors embedded in the flexible design of the ring described here. Embodiments that use technology that does not reduce melanin bias are undesirable in comparison to the melanin bias reducing technology described in this document. However, it may be desirable for use in implementing the wireless system, further described in this document, while FDA approvals for the melanin bias reduction technology is achieved.

Further, other example embodiments include an inertial measurement unit (IMU), accelerometer, gyrometer, altitude sensor, angular velocity sensor, multi-axis inertia sensor, global positioning sensor (GPS), global navigation satellite system (GNSS), or magnetometer to create motion or location data utilized for on board subject location tracking, fall detection, and bed/chair alarming. In some example embodiments, the motion data is used to alert medical staff if a patient attempts to stand from a toilet or leave an area without assistance. Further example embodiments utilize the motion and location data for finger and hand tracking, for example, but not limited to, augmented reality (AR), virtual reality (VR), extended reality (XR), and American Sign Language (ASL) detection purposes.

The embodiment described in FIG. 33 primarily describes an example wireless pulse oximetry ring embodiment with a self-contained power source displaying its data on an external screen. A further example embodiment includes a small low power screen, for example, e-ink or OLED to display pulse oximetry data directly on the ring. Further example embodiments include a UI connection for an external or wrist worn screen as shown in FIG. 19 and FIG. 21. These example embodiments are implemented with the hardware in the ring and only utilize an external screen controlled by the ring hardware. Another example embodiment with an external screen removes the PCBs and power supply from the ring and locates these in the external screen to provide a larger power source for the screen and to utilize lower cost rigid PCBs in the external screen. Further example embodiments utilize reduced component counts in the ring to connect to an external screen or patient monitoring system in a similar fashion as current pulse oximeters. These embodiments provide a solution to an inexpensive disposable ring, and only include for example the emitter and detector.

Further an example of a reduced component embodiment which connects to a patient monitoring system similar to current pulse oximeters include, for example, only the LED emitters, which is controlled by the system it is connected to or in some example embodiments includes the LED emitters with minimal constant current control and switching logic. Further an example of a reduced component embodiment which connects to a patient monitoring system similar to current pulse oximeters include, for example, only the photodiode and transmits the small current signals directly to the system it is connected to. However, this embodiment is highly susceptible to noise due to the transmission of small current signals, and therefore often requires extensive shielding. Further an example of a reduced component embodiment which connects to a patient monitoring system similar to current pulse oximeters includes, for example, the photodiode with a transimpedance stage to directly send the analog voltage signals to the system it is connected to.

However, this embodiment is also susceptible to noise due to the transmission of analog signals, and therefore often requires extensive shielding. Further an example of a reduced component embodiment which connects to a patient monitoring system similar to current pulse oximeters includes, for example, the photodiode with a transimpedance stage and digital conversion stage to directly send the digital intensity signals to the system it is connected to. These example embodiments represent a drop-in replacement for current pulse oximeters with the included melanin bias reduction optical hardware and ring embodiment described in this document. In other words, these example embodiments are comparable in component count and price to current disposable pulse oximeters and function with the same electrical connections to current patient monitoring systems while including the additional melanin bias reduction and ring functionality. Additionally, example embodiments include motion and location tracking capabilities, as further described in this document to further reduce the number of monitoring devices required for a subject.

FIG. 34A-34B shows two exploded views diagrams of the example design of a flexible ring embodiment of a pulse oximeter, including the internal components' arrangement to allow for flexible adjustment as shown in FIG. 33. FIG. 34A shows one example diagram of the internal cable flexor tract that provides flexible adjustment, and FIG. 34B shows another example diagram of the internal cable flexor tract that provides flexible adjustment. 3401 and 3412 represent the outer case of the example ring embodiment as described in 3301. 3402 and 3413 represent the flexible PCB in the example ring embodiment as described in 3302. 3403 and 3414 represent the detector in the example ring embodiment as described in 3303. 3404 and 3415 represent the battery in the example ring embodiment as described in 3304. 3405 and 3416 represent the flexible PCB substrate for mounting the emitter in the example ring embodiment as described in 3305. 3406 and 3417 represent the emitter in the example ring embodiment as described in 3306. 3407 and 3418 represent the flexible cover that separates the internal components from the appendage the ring is worn on in the example ring embodiment as described in 3307. 3408 and 3419 represent cutaway views of two example embodiments of the flexible wire/ribbon cable chase on the inside of 3407 and 3418, respectively, (also 3307).

The chase is designed to allow the electrical interconnects between ring components to expand or contract as needed. The PCBs and batteries do not stretch but rather the ring casing stretches around these components. 3409 and 3420 represent the back or inside of 3407 and 3418, respectively, (also 3307). The serpentine pattern shown in the example drawings is created directly on the back of 3407 and 3418, respectively, (also 3307). 3410 and 3421 show the location the electrical interconnection ribbon cable or wires are placed. This chase provides a safe method for electrical interconnects where enough cable is provided to be stretched into a straight line without damage as the ring flexes to expand or contract, which moves the serpentine on the back of 3407 and 3418, respectively, (also 3307) in a linear method as shown by arrows 3411 and 3422, respectively. The example embodiment 3408 includes additional edges in the serpentine, as shown in the example drawing, when compared with 3419. The additional edges in 3408 provide additional protection of the electrical interconnects when moving, while sacrificing some flexibility when compared with 3419. The wire interconnect methods and component placements and designs in the example drawing are to be considered an example and other methods of assembling the ring within the scope of this document will be obvious to those in the field.

FIG. 35 shows a diagram of another example design of a flexible ring embodiment of a pulse oximeter including the internal components arrangement to provide flexible adjustment. The example embodiment shown in FIG. 35 is similar to the example embodiment shown in FIG. 33 with an example of differences in the configuration of the PCBs and battery. Much of the information discussed in this example drawing (FIG. 35) is similar to that of FIG. 33. 3501 is the outer case of the ring, as described in FIG. 20. 3501 is made out of a flexible material in example embodiments that require a stretchable band for either a snug fit or to provide an adjustable ring for different size appendages. Additionally, a stretchable band provides flexibility in the ring for swelling of the appendage for medical reasons without reducing circulation in the appendage.

Materials a flexible embodiment are made of are described in FIG. 20, and include, but are not limited to, flexible rubber, TPU, elastic, EVA foam, VELCRO strapping, medical grade rubbers, foams, and plastics, flexible breathable TEGADERM film, silicon, and/or other types of flexible rubbers or plastics known in the field. Other example embodiments of 3501 include rigid rings similar to a wedding band. In rigid embodiments, 3501 is made of plastics, silicones, metals, rigid rubbers, medical grade materials, and/or other band materials known in the field.

As mentioned in FIG. 20, other example ring embodiments are coated, treated, and/or designed with antimicrobial surface, finishes, and/or chemicals. FIG. 35 shows an example layout of the components found in the inner chamber of the ring previously described in 2002 and FIG. 22. FIG. 35 shows an example embodiment in which there are two batteries 3507 and 3504 and two PCBs 3502 and 3505, each taking up a fourth of the ring. This design keeps in mind the flexible requirement of example ring embodiments by ensuring the battery does not surround the ring. This example embodiment differs in design from the example shown in FIG. 33 and provides additional flexibility by separating the most rigid component, the battery 3507 and 3504 (also 3304), into two separate internally shiftable parts. This embodiment provides the ring additional contour flexibility to better match the appendage it is worn on. Other example embodiments in which the ring's outer case is rigid, batteries 3504 and 3507 and PCBs 3502 and 3505 take on different configurations. One example of these additional configurations includes batteries 3504 and 3507 that follow the circumference of the ring to provide additional battery capacity in a non-rigid form.

Additional configurations of PCBs 3502 and 3505 and batteries 3504 and 3507 are possible and will be obvious to those in the field. PCBs 3502 and 3505 include the on-board electronic components required for the ring to function. These PCBs split the components required between them based on functional design, and in an example embodiment include duplicate components for controlling both batteries. One of these components shown is the detector 3503. In the embodiment shown, detector 3503 is an analog photodiode, which outputs a current signal based on the light impinged on the detector from the emitter 3506. Other example embodiments use a detector 3503 that includes a transimpedance amplifier on one integrated circuit to reduce the component count required and to reduce energy consumption.

Further, additional example embodiments use a detector 3503 that includes all required circuits to produce a digital signal, therefore eliminating the need for an ADC (analog to digital converter), and therefore reducing noise interference between the detector and the microcontroller (MCU or processor). PCBs 3502 and 3505, also include all additional hardware required for the pulse oximeter to function. One example embodiment of the PCBs 3502 and 3505 split between them at least one MCU (microcontroller or processor), which runs the main software program, the power management hardware, the DAC (digital to analog converter) for the emitter constant current controller, the ADC (analog to digital converter), the transimpedance amplifier, filtering, gain control, battery charging controller, battery gauge hardware, and user interference connection.

Example embodiments that include wireless capabilities also include wireless components on PCBs 3502 and 3505. Example embodiments of charging methods include, but are not limited to, magnetic charging, inductive charging, micro USB, mini USB, USB-C, waterproof micro USB, waterproof mini USB, waterproof USB-C, QI wireless charging, and charging contacts on a charging cradle. Example embodiments that use inductive or QI wireless charging include a wireless charging coil, not shown. Further example embodiments that use charging methods with external contacts are not visualized in the example embodiment drawing. One example embodiment uses Nordic MCU nrf52832 or nrf52840 due to the onboard BLUETOOTH Low Energy (BLE) system on chip (SoC), as well as BLUETOOTH mesh, THREAD, ZIGBEE, 802.15.4, ANT and 2.4 GHz proprietary SoC stacks. Further, other example embodiments use other MCUs with other wireless capabilities as further described in FIG. 22 and further expounded on in FIG. 27.

Other example embodiments use digital methods for filtering and amplification to reduce component counts as described in FIG. 22 and FIG. 23. Further, other example embodiments use other component count reducing methods such as an MCU with a built-in ADC and/or DAC, as well as other LED control methods. Other example embodiments additionally use software-based battery gauge and charging control methods to further reduce component counts. One example embodiment uses wafer level components on flexible PCBs 3502 and 3505 in order to reduce the size of the required components. Other example embodiments switch the positions of emitter 3506 and detector 3503. Further, other example embodiments have multiple emitters and detectors spaced around the ring to provide more options for light transmission through the appendage. This is used to reduce issues that arise with poor contact of some emitters and detectors depending on the position of the ring.

Additionally, other example embodiments use reflective technology for the detector and emitter. In these example embodiments, the second PCB section 3505, including the emitter 3506, is not necessary as the detector and emitter are placed near each other on the same PCB 3502. In other example embodiments, PCB 3505 is necessary even when reflective technology is used for the optics in order to provide the additional component space required. Further, other example embodiments include a self-contained photoplethysmography (PPG) sensor integrated circuit (IC), instead of detector 3503 and emitter 3506.

An example melanin bias reducing PPG sensor is described further in this document. However, as described further in FIG. 35 an off the shelf sensor that does not reduce melanin bias is used in some example embodiments. When additional component space is not required, PCB 3505 only provides a substrate to solder emitter 3506 to, as well as a way to securely electrically connect to PCB 3502. One example embodiment uses flexible ribbon cable to connect the two PCBs, while other example embodiments use small individual wires or twisted pairs to connect the two PCBs. Emitter 3506 represents at least two light sources, as further described in this document. An example embodiment uses separate LEDs configured next to each other, while other example embodiments use a single LED body including separate emitters that can be controlled independently. Further example embodiments use other types and styles of light sources as described in this document.

Additional example embodiments that use a single LED body with separate emitters and do not use PCB 3505 as a mounting substrate, have a ribbon cable, individual wires, or twisted pairs coming directly off the LED body to connect to PCB 3502. 3504 and 3507 are the batteries used to power the electronic components embedded in the ring. In the example embodiment shown, 3504 and 3507 each occupy a fourth of the ring to provide the flexibility of the ring to stretch around the batteries for different sized appendages. Other example embodiments use other battery configurations to provide more or less flexibility/rigidity as required. The battery 3504 and 3507 is described further in FIG. 22. However, in the ring configuration of the pulse oximeter, the battery is designed to follow the curved contour of the ring shape as shown in the example embodiment. Many example embodiments use a standard LiPo battery shaped with a ring in mind.

However, these batteries have very low tolerance to flexing, therefore other example embodiments use a solid-state battery or battery/power source that is highly flexible. In the example embodiment shown, batteries 3504 and 3507 are connected directly to PCB 3502 and PCB 3505 on one end, while the other end is not attached to provide the expandable function of the ring to fit multiple sized appendages. In other example embodiments the batteries use similar electrical connection methods at emitter 3506, such as, but not limited to, ribbon cables, wires, and/or twisted pairs. 3508, represents the flexible cover that separates the internal components from the appendage the ring is worn on. The cover has cutouts for detector 3503 and emitter 3506.

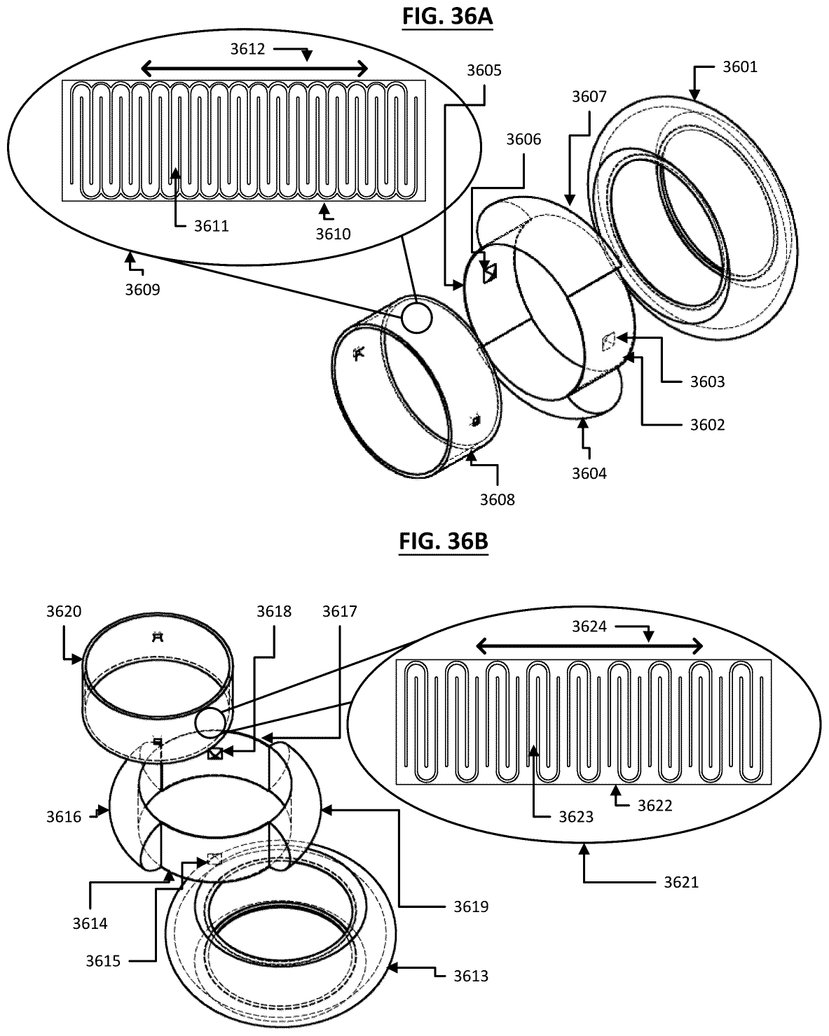

The detector and emitter are designed to stay in fixed positions while the ring expands or contracts around them with the other ring components shifting as necessary. This process is further described in FIG. 36. 3508 is designed using the same materials as described elsewhere in this document for 3501 and FIG. 20. All embodiments described in this section have the emitter 3506 and detector 3503 configured as described elsewhere in this document to include melanin bias reduction optical properties. Further, other example embodiments include a detector 3503 and emitter 3506, as known in the field, and are currently on the market. These example embodiments include optics that do not reduce melanin bias and are built using current technology and off the shelf parts, including, but not limited to, on the market PPG sensors embedded in the flexible design of the ring described here. Embodiments that use technology that does not reduce melanin bias are undesirable in comparison to the melanin bias reducing technology described in this document.

However, it may be desirable for use in implementing the wireless system, further described in this document, while FDA approvals for the melanin bias reduction technology is achieved. Further, other example embodiments include an inertial measurement unit (IMU), accelerometer, gyrometer, altitude sensor, angular velocity sensor, multi-axis inertia sensor, global positioning sensor (GPS), global navigation satellite system (GNSS), or magnetometer to create motion or location data utilized for on board subject location tracking, fall detection, and bed/chair alarming. In some example embodiments the motion data is used to alert medical staff if a patient attempts to stand from a toilet or leave an area without assistance. Further example embodiments utilize the motion and location data for finger and hand tracking, for example, but not limited to, augmented reality (AR), virtual reality (VR), extended reality (XR), and American Sign Language (ASL) detection purposes.

The embodiment described in FIG. 35 is an example wireless pulse oximetry ring embodiment with a self-contained power source displaying its data on an external screen. A further example embodiment includes a small low power screen for example e-ink or OLED to display pulse oximetry data directly on the ring. Further example embodiments include a UI connection for an external or wrist worn screen as shown in FIG. 19 and FIG. 21. These example embodiments are implemented with the hardware in the ring and only utilize an external screen controlled by the ring hardware. Another example embodiment with an external screen locates the PCBs and power supply in the external screen, rather than in the ring, to provide a larger power source for the screen and to utilize lower cost rigid PCBs. Further example embodiments utilize reduced component counts in the ring to connect to an external screen or patient monitoring system in a similar fashion as current pulse oximeters.

These embodiments provide a solution to an inexpensive disposable ring and only include, for example, the emitter and detector. Further, these example embodiment's emitters include, for example, only the LED emitters, which is controlled by the system it is connected. In other example embodiments, the ring includes the LED emitters with minimal constant current control and switching logic. Further example embodiment's detector include, for example, only the photodiode and transmits the small current signals directly to the system it is connected to. However, this embodiment is highly susceptible to noise due to the transmission of small current signals, and therefore often requires extensive shielding. Further example embodiment's detector includes, for example, the photodiode with a transimpedance stage to directly send the analog voltage signals to the system it is connected to. However, this embodiment is also susceptible to noise due to the transmission of analog signals, and therefore often requires extensive shielding. Further example embodiment's detector includes, for example, the photodiode with a transimpedance stage and digital conversion stage to directly send the digital intensity signals to the system it is connected to.

FIGS. 36A-36B show two exploded view diagrams of the example design of a flexible ring embodiment of a pulse oximeter, including the internal components arrangement to allow for flexible adjustment as shown in FIG. 35, in accordance with the invention. FIG. 36A shows one example diagram of the internal cable flexor tract that provides flexible adjustment, and FIG. 36B shows another example diagram of the internal cable flexor tract that provides flexible adjustment. 3601 and 3613 represent the outer case of the example ring embodiment as described in 3501. 3602 and 3614 represent the flexible PCB in the example ring embodiment as described in 3502. 3603 and 3614 represent the detector in the example ring embodiment as described in 3503. 3604 and 3616 represent the battery in the example ring embodiment as described in 3504. 3605 and 3617 represent the second flexible PCB in the example ring embodiment as described in 3505. 3606 and 3618 represent the emitter in the example ring embodiment as described in 3506. 3607 and 3619 represent the second battery in the example ring embodiment as described in 3507. 3608 and 3620 represent the flexible cover that separates the internal components from the appendage the ring is worn on in the example ring embodiment as described in 3508. 3609 and 3621 represent cutaway views of two example embodiments of the flexible wire/ribbon cable chase on the inside of 3608 and 3620, respectively, (also 3508).

The chase is designed to allow the electrical interconnects between ring components to expand or contract as needed. 3610 and 3622 represent the back or inside of 3608 and 3620, respectively, (also 3508). The serpentine pattern shown in the example drawings is created directly on the back of 3608 and 3620, respectively, (also 3508). 3611 and 3623 show the location the electrical interconnection ribbon cable or wires are placed. This chase provides a safe method for electrical interconnects that have enough cable to be stretched into a straight line without damage as the ring flexes to expand or contract, which moves the serpentine on the back of 3608 and 3420, respectively, (also 3508) in a linear method as shown by arrows 3612 and 3624, respectively. The example embodiment 3609 includes additional edges in the serpentine, as shown in the example drawing, when compared with 3621. The additional edges in 3609 provide additional protection of the electrical interconnects when moving, while sacrificing some flexibility when compared with 3621. The wire interconnect methods, and component placements and designs in the example drawing are to be considered an example and other methods of assembling the ring within the scope of this document will be obvious to those in the field.

### Self Contained Photoplethysmography (PPG) Sensor Integrated Circuit

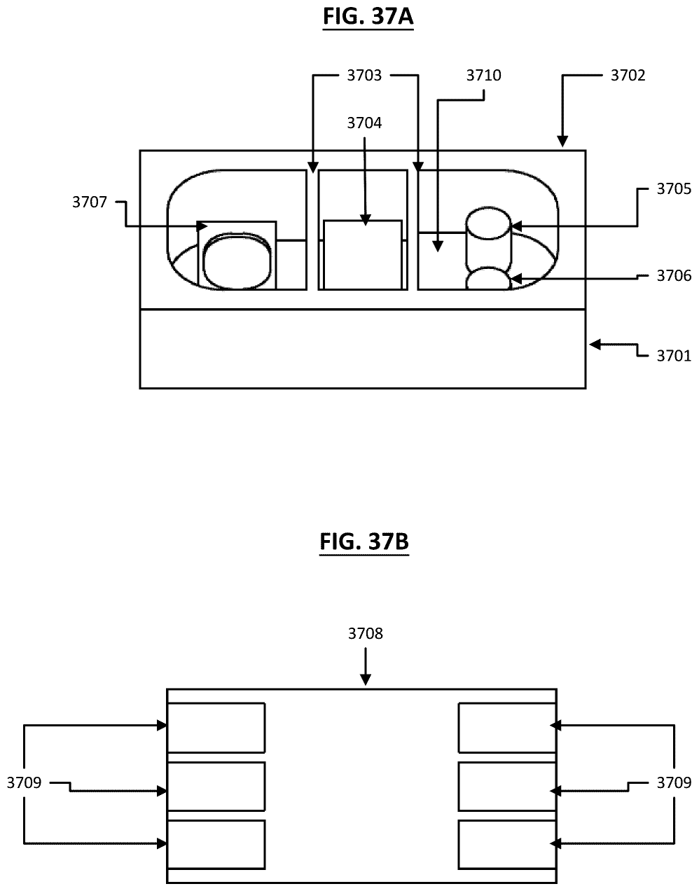

FIGS. 37A-37B show side and bottom view diagrams of an example design of a single chip PPG (Photoplethysmography) sensor including melanin bias reducing technology as a drop in smart device and electronics sensor. Other example uses for this sensor include, but are not limited to, use in hospital grade and consumer grade pulse oximeters, exercise equipment that detects a heart rate or SpO₂ levels, smart watches, hobbyist sensors, exercise devices, and/or embedded healthcare devices. Another example use for this device includes, but is not limited to, use in reflective pulse oximeter designs, such as, for example, tape on pulse oximetry monitors. 3701 represents the outer shell case of the PPG sensor.

One example embodiment of the sensor measures about 6 mm by 3 mm around the outside of 3701 with a height of about 1.5 mm. 3702 represents the clear protective cover that covers the microscopic components inside 3701 and also provides a clear path for light to leave the emitters and reflect back to the detector. This clear protective cover in many usage cases is in direct contact with patients and is therefore created with materials known in the field that are safe for disinfection and cleaning without compromising the optical properties. 3703 represents an opaque divider that prevents light from directly passing from the emitters to the detector without first reflecting through the subject.

In other example embodiments only one divider section is necessary between emitters 3705 and 3706 and the other components in the case. 3704 is the wafer that includes all transistors and other components required to control the emitters, receive intensity data from the detector, process this information, and provide a communication interface on pins 3709 to connect the sensor to a microcontroller (MCU or processor). Other example embodiments include analog filtering components on wafer 3704, while other example embodiments perform this filtering digitally on the processor embedded on wafer 3704. Further, other example embodiments do not provide filtering and present raw data on the communication interface on pins 3709. 3705 and 3706 represent the dies for the example LED emitters.

Other example embodiments include additional emitters, as further described in this document, to measure other types of physiological data and/or to provide additional accuracy to pulse oximeter measurements. Further example embodiments use other types and styles of light sources as described in this document. 3710 represents the reflective backing in the emitter chamber of the sensor that focus the emitter beams toward the subject and away from the inside of the case 3701. 3707 represents the detector. In the embodiment shown, detector 3707 is an analog photodiode which outputs a current signal based on the light impinged on the detector from the emitters after the light passes through or reflects through the appendage of the subject.

Other example embodiments use a detector 3707 that includes a transimpedance amplifier on one integrated circuit to reduce the component count required and to reduce energy consumption. Further, additional example embodiments use a detector 3707 that is part of wafer 3704 and therefore includes all required circuits on one wafer to produce a digital signal, therefore eliminating the need for a separate ADC (analog to digital converter) on the wafer. All embodiments described in this section have the emitters 3705 and 3706 and detector 3705 configured as described elsewhere in this document to include melanin bias reduction optical properties. The example embodiment shown does not show the wire bonding used to interconnect portions of the on-board components and to connect the wafer to the pads. 3708 is also representative of case 3701, as shown in the bottom view of the sensor in FIG. 37B, and has been described above. 3709 represents the communication interface to communicate digitally with an external MCU (microcontroller or processor) or with a patient monitoring system interface.

The example embodiment shown represents a 6 pin interface including supply power, ground, and an SPI interface (Master in Slave Out [MISO], Master out Slave In [MOSI], Clock [SCK], and Chip Select [CS]). Other example embodiments use other digital communication interfaces known to those in the field, such as, but not limited to, I²C, parallel, and proprietary methods. Further other example embodiments use analog communication and control methods, which remove the need for wafer 3704. An example of analog communication includes, but is not limited to, an interface which includes direct photodiode access, ground, supply power, and direct access to control the emitters. Another example embodiment uses that same interface, however uses a photodiode design that includes a transimpedance amplifier and therefore outputs an analog voltage signal in relationship to the photodiode detected intensity, instead of the small signal current signal directly output by the photodiode. In embodiments that use analog style outputs and controls, proper shielding is required to ensure minimal signal loss and interference.

### Heart Telemetry System

Heart telemetry (sometimes broadly defined as “telemetry”) is the system and devices used to measure and monitor patient physiological information. Heart telemetry specifically refers to the ECG portion of telemetry. Some ECGs use 10 cables to determine 12 electrical views or leads of the heart. This method is accomplished by placing 6 electrode pads across the chest, labeled V1 to V6. V1 and V2 are placed on either side of the sternum on or near the 4^{th} rib. V4 is placed on the apex of the heart which is at the tip of the left and right ventricles and is often on or near the 5^{th} rib. V3 is placed halfway between V2 and V4. V5 and V6 are placed horizontally laterally from V4 and often fall between ribs 5 and 6 or on rib 6. It is important not to place V5 and V6 upward toward the axilla (the area directly under the shoulder joint). Additionally leads are placed on each limb, which accounts for the additional 4 electrode pads. However, when calculating the ECG waveform, the right leg is often considered a reference ground or earth electrode which is used to reduce noise in the overall calculation.

When measuring the difference electrically (potential difference) between different leads and using the ground to reduce noise, it is possible to reproduce a graphical representation of the 3-dimensional electrical activity of the heart. The limb electrodes are grouped into bipolar and unipolar leads, where the bipolar leads are a direct potential difference between the two electrodes, and the unipolar leads register electrical activity directed towards or located below the electrode. In other words, in bipolar measurements the potential difference between the pair of electrodes is amplified by a signal differential channel, and in a unipolar measurement the output signal is calculated by using the input of a single electrode and amplified against a reference. This reference can be a common electrode, or an internally calculated reference potential including multiple electrode signals. Lead 1 is referred to as the right arm and left arm, lead 2 is referred to as the right arm and left leg, and lead 3 is referred to as the left arm and left leg for the potential differences. The right leg is considered a ground electrode as previously mentioned. The unipolar leads are referred to as augmented leads and are the potential difference between one of the three electrodes and the estimated zero potential, which is calculated from the other two electrodes combined. These leads are often called aVR lead for the right arm, aVL lead for the left arm, and aVF lead for the left leg.

The lead on the legs and feet form what is known as Einthoven's triangle and further explains why there are 6 leads when only 4 electrodes are used. These leads are able to be plotted on a vertical plane of the heart with zero degrees (0°) starting on the x axis and moving clockwise around the heart. Following this vertical plane model of the heart 0° is lead 1, 60° is lead 2, 90° is lead aVF, 120° is lead 3, −30° is lead aVL, and −150° is lead aVR. Electrodes at V1 to V6 provide the additional set of unipolar leads in which the potential difference is calculated between the chest electrode and an estimated zero potential which is often derived from the average potential from the 3 limb leads. V1 to V6 provide data on the horizontal plane of the heart. When these signal planes are combined, a 2-dimensional multi-graph of the heart is generated which provides a full 3-dimensional visual representation of the heart. These views are the inferior view using leads 2, 3, and aVF, the anterior view using leads 1, aVL, V1, V2, and V3, the septal view using leads V3 and V4, and the lateral view using leads V4, V5, and V6. When the heart produces an electrical signal for the leads to record, depolarization toward a lead produces a positive deflection on the output graph and depolarization away from a lead produces a negative deflection on the output graph.

Depolarization is considered the process by which the electrical current passes through the heart muscle to change it from its resting polarized state as it contracts. Repolarization is the process in which the heart muscle returns to rest. As the heart moves from depolarization through repolarization it produces an ECG waveform known as the ECG trace, which is comprised of 3 waves called P, QRS complex, and T.

The following describes a normal ECG trace. The P wave is the small deflection that occurs during atrial depolarization. The atrium is the upper portion of the heart and the P wave often starts on the right side (from the perspective of the subject) and travel toward the left atrium. The PR interval is the total time between the start of the deflection in the P wave to the start of the first deflection of the QRS complex wave. The QRS complex wave includes 3 waves which represent ventricular depolarization. The smaller Q wave represents the depolarization of the interventricular septum. The interventricular septum is center mass of the heart between the right and left ventricles. The R wave is often a large deflection and represents the depolarization of the main mass of the ventricles. The S wave is smaller than the R wave and representants the last depolarization of the ventricles at the base of the heart.

At the end of the QRS interval/wave the J point represents the point where the QRS complex joins the ST segment and represents the approximate point when depolarization ends and repolarization begins. The ST segment is the time between the QRS complex and the start of the T wave. The ST segment is a period of zero potential between ventricular depolarization and repolarization. In other words, the ST segment has no net difference in electrical potential. The Qt interval is the time from the start of the QRS complex wave to the end of the T wave. The T wave represents the ventricular repolarization or the heart beginning to go back to its relaxed state. In some ECG waveforms, an additional small U wave after the T wave can be seen as part of the final repolarization of the heart muscles. The U wave, however, is not generally discussed in basic ECG waveform literature since it is not always seen in ECG waveforms and the direct cause of the U wave is considered elusive; however, abnormal U waves can be an indicator of various health concerns.

Other ECG systems use 5 electrodes to function. These systems are one of the most commonly found in hospital long term monitoring systems. They include a left arm, right arm, left leg, and right leg electrode as well as a chest electrode in one of the V positions previously mentioned. The V1 electrode placement is often the most common position as it offers better arrhythmia monitoring, however it can be placed in any V position as needed. These systems display the bipolar leads 1, 2, and 3 previously discussed, as well as the single unipolar V lead on the monitoring system. Additionally, another commonly found ECG system in hospital long term monitoring uses 3 electrodes to function. ECG 3 electrode systems include a left arm, right arm, and left leg electrode and some systems include a 4^{th} electrode as a ground reference to reduce noise, or as a common mode rejection electrode. In these systems best results are often achieved when the electrodes are not placed on the specified limbs, but are place on the chest and abdomen equidistant from the heart. The monitor in these systems displays the leads 1, 2 and 3 which are the bipolar leads previously discussed. In most hospital monitoring systems only lead 2, which is the right arm to left leg lead, is continuously displayed on the patient monitoring system.

Additionally, some systems also calculate the respiration rate displayed on patient monitoring systems using the ECG leads using signals from diaphragm moment, while other systems use the pleth graph from pulse oximetry to derive the respiration rate from the plethysmograph. Described below are embodiments that apply the traditional methods of ECG to compact fully wireless ECG and patient monitoring methods.

FIG. 27 shows an example embodiment of a wireless heart telemetry system. User 2701 wears heart monitor pads 2702-2704, which wirelessly sends data to patient monitoring system 2705. The example locations of monitoring pads 2702-2704 are meant for demonstration purposes and are not to be considered the only locations or quantity of pads required for heart telemetry monitoring. In the example shown, 2702-2704 utilize disposable snap on or clip on pads similar to those used in current heart telemetry systems, which are in contact with the patient skin. Snapped or clipped onto these pads are the reusable devices, which include the hardware required to wirelessly transmit the heart telemetry data to the patient monitoring system 2705.

This hardware includes for example, but is not limited to, filtering, MCU, wireless SoC, battery, and isolation technology. The signals from 2702-2704 must be synchronized, in order for the system to work properly using, for example, time of flight calculations and/or synchronized time stamping. One example embodiment of the system, described in FIG. 27, utilizes independent grounds for 2702-2704, while other example embodiments use an interconnected and centralized ground. One example embodiment of FIG. 27 utilizes BLUETOOTH as its wireless communication method, while other example embodiments use other wireless communications, such as, but not limited to, WI-FI, LORA, or other proprietary wireless communications methods. 2705, in the example embodiment shown in FIG. 27, is a bedside patient monitoring system.

Other example embodiments use bedside systems connected to hospital wide patient monitoring systems, pocket patient monitoring systems connected to bedside and/or hospital wide patient monitoring systems, independent pocket patient monitoring systems, smart device patient monitoring systems, portable patient monitoring systems, or hospital wide patient monitoring systems, instead of bedside patient monitoring system 2705. The portion of the system worn on the user 2701 in FIG. 27 is designed to be waterproof for continuous patient monitoring, including damp locations, such as the bath or shower.

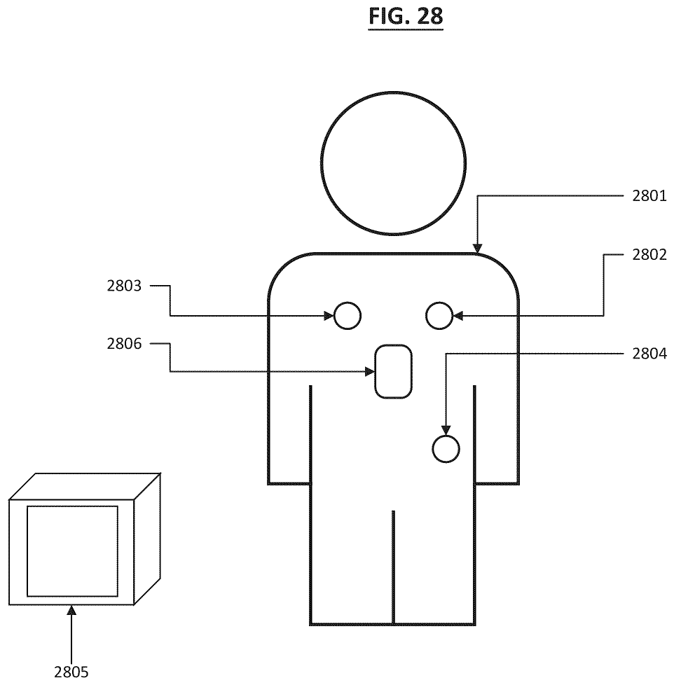

FIG. 28 shows another example embodiment of a wireless heart telemetry monitoring system. User 2801 wears heart monitor pads 2802-2804, which wired or wirelessly sends data to wearable patient monitoring base point 2806. The example locations of monitoring pads 2802-2804 are meant for demonstration purposes and are not to be considered the only locations or quantity of pads required for heart telemetry monitoring. In the example shown, 2802-2804 utilize disposable snap on or clip on pads, which are in contact with the patient skin, similar to those used in current heart telemetry systems. Snapped or clipped onto these pads are the reusable devices, which include the hardware required to wired or wirelessly transmit the heart telemetry data to the wearable base point 2806. In wireless embodiments, this hardware, for example, includes, but is not limited to, filtering, MCU, wireless SoC, battery, and isolation technology. The wireless signals from 2802-2804 must be synchronized, in order for the system to work properly using, for example, time of flight calculations and/or synchronized time stamping.

One example embodiment of the wireless system, described in FIG. 28, utilizes independent grounds for 2802-2804, while other wireless example embodiments use an interconnected and centralized ground. In example wired embodiments, the signals from pads 2802-2804, including grounds, are connected directly to 2806, and 2806 includes the hardware described above in wireless embodiments. One example embodiment of wireless connectivity between 2802-2804 and 2806 utilizes BLUETOOTH as its communication method, while other example embodiments use other wireless communication methods, such as, but not limited to, LORA, NFC, RFID, or other proprietary wireless communication methods.

One example embodiment of 2806 utilizes BLUETOOTH as its wireless communication method to patient monitoring system 2805, while other example embodiments use other wireless communications, such as, but not limited to, WI-FI, LORA, or other proprietary wireless communication methods. In wireless hospital patient monitoring systems, 2805 uses similar communication methods, as described above, for wearable base point 2806. 2805, in the example embodiment shown in FIG. 28, is a wired or wireless bedside patient monitoring system.

Other example embodiments use bedside systems connected to hospital wide patient monitoring systems, pocket patient monitoring systems connected to bedside and/or hospital wide patient monitoring systems, independent pocket patient monitoring systems, smart device patient monitoring systems, portable patient monitoring systems, or hospital wide patient monitoring systems, instead of bedside patient monitoring system 2805. 2805 utilizes wired or wireless methods of communication for all example embodiments listed above.

Other example embodiments of 2805 use wired TCP/IP or other types of ethernet connections to connect to hospital patient monitoring systems, as described above. 2806, in the example embodiment shown, does not include a screen, in order to save battery life, while some other example embodiments include a screen for convenience. The example placement of 2806 is meant for demonstration purposes and is not to be considered the only location that is used. For example, patients that have pace makers or other implanted devices near or around the area shown, may not be able to have a wireless device in close proximity to their implant, due to interference concerns. In situations in which there is a concern for interference with an implant, 2802-2804 can be connected, via wired methods to 2806, and 2806 can be placed in a non-interfering location on the body. 2806, in the example embodiment shown in FIG. 28, is attached to user 2801, via medical grade adhesives, such as, but not limited to, temporary skin glue, tapes, and/or TEGADERM. In other example embodiments, 2806 is a pocket worn or belt clip worn device.

The portion of the system worn on the user 2801 in FIG. 28 is designed to be waterproof for continuous patient monitoring, including damp locations, such as the bath or shower. Further, other example embodiments are also intrinsically sealed for continuous monitoring in hazardous situations such as, for example, in-patient settings and also in the field on emergency personnel as a real time vitals monitoring system. Other example embodiments use for example, but not limited to, cellular or emergency digital radio interfaces to transmit emergency personnel and military vital information using the heart telemetry system described herein. Further, other example embodiments have reflective melanin bias reducing pulse oximetry systems, as described previously in this document, built into the wearable base point 2806 in example embodiments where the base point is attached directly to the skin.

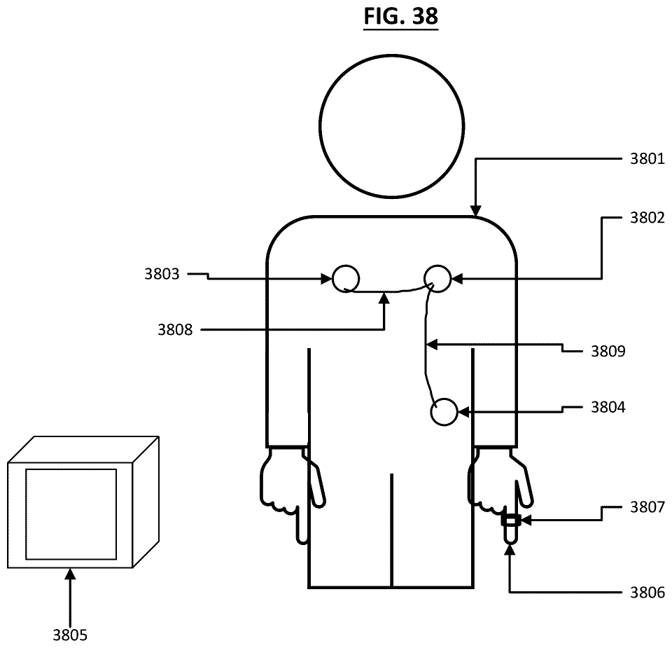

FIG. 38 shows an example embodiment of a wireless heart telemetry system and additionally includes wireless pulse oximetry in an example wireless ring embodiment as a patient monitoring system, as further described in another section of this document. User 3801 wears heart monitor pads 3802-3804, which wirelessly sends data to patient monitoring system 3805, via a central wireless heart telemetry device 3802. The example locations of monitoring pads 3802-3804 are meant for demonstration purposes and are not to be considered the only locations or quantity of pads required for heart telemetry monitoring. As previously discussed, this example embodiment represents a minimum 3 electrode Einthoven's triangle monitoring arrangement.

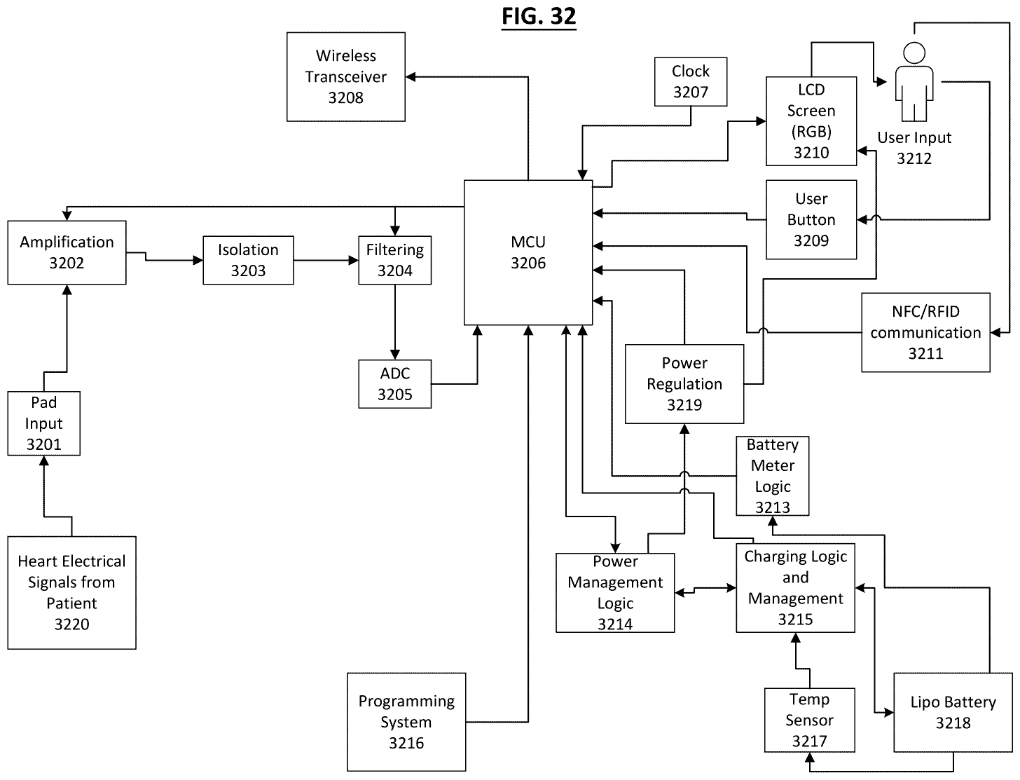

However, additional example embodiments in this arrangement utilize an additional electrode to assist in noise reduction and in some example embodiments to provide a more accurate respiration rate. In the example shown, 3802-3804 utilize disposable snap on or clip on pads similar to those used in current heart telemetry systems, which are in contact with the patient skin. Snapped or clipped onto pad 3802 is the reusable wireless heart telemetry device, which includes the hardware required to wirelessly transmit the heart telemetry data to the patient monitoring system 3805. This hardware includes for example, but is not limited to, filtering, MCU, wireless SoC, battery, and isolation technology, as further described in FIG. 32. The signals from reusable pads 3803-3804 are transmitted using cables 3808 and 3809 respectively to the central heart telemetry device clipped to pad 3802.

Although this example embodiment, still utilizes cables to interconnect the reusable pads, it provides significant comfort and ease of use by including all cables under the subject's clothing or hospital gown, and prevents a large number of cables to be connected to a device worn externally to the patient. One example embodiment of FIG. 38 utilizes BLUETOOTH (classic or low energy) as its wireless communication method, while other example embodiments use other wireless communications, such as, but not limited to, WI-FI, LORA, ZIGBEE, or other proprietary wireless communications methods. 3805, in the example embodiment shown in FIG. 38, is a bedside patient monitoring system.

Other example embodiments use bedside systems connected to hospital wide patient monitoring systems, pocket patient monitoring systems connected to bedside and/or hospital wide patient monitoring systems, independent pocket patient monitoring systems, smart device patient monitoring systems, portable patient monitoring systems, or hospital wide patient monitoring systems, instead of bedside patient monitoring system 3805. The portion of the system worn on the user 3801 in FIG. 38 is designed to be waterproof for continuous patient monitoring, including damp locations, such as the bath or shower. Additionally, the example embodiment shown in FIG. 38 shows the example wireless ring pulse oximeter embodiment, 3807, worn on the subject's finger, 3806 to provide the patient monitoring system with the additional physiological information, including, but not limited to SpO₂, pleth graph, and heart rate (calculated from the pleth graph in addition to the ECG calculated heart rate).

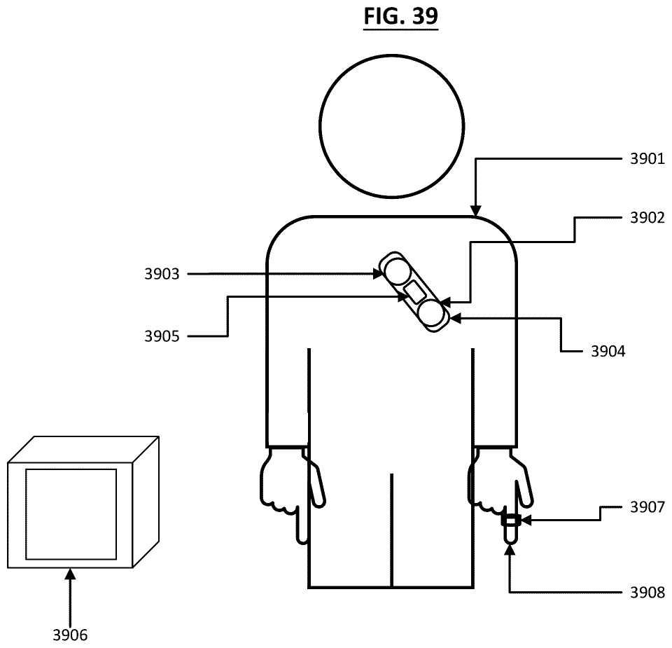

FIG. 39 shows an example embodiment of a wireless heart telemetry monitoring system with an example embodiment of wireless patient/subject worn conductive pads that include a central wireless heart telemetry electrode with an example straight two pad clip, and includes an example ring embodiment for combined wireless pulse oximetry, as a patient monitoring system. User 3901 wears heart monitor pads 3902-3903, which wirelessly sends data to patient monitoring system 3906, via a central wireless heart telemetry device 3905. The example locations of monitoring pads 3902-3903 are meant for demonstration purposes and are not to be considered the only locations or quantity of pads required for heart telemetry monitoring. As previously discussed, this example embodiment represents a single 2 electrode lead in the 3 electrode Einthoven's triangle monitoring arrangement.

This arrangement produces the ECG graph for ECG lead 2, which is most commonly selected on hospital monitoring systems; however, additional pad locations are required for most advanced medical decisions and nurse monitoring stations. One example embodiment adds the additional pads when necessary, utilizing wired or wireless methods, further described in other figures in this document. In the example shown, 3902-3903 utilizes disposable snap on or clip on pads, similar to those used in current heart telemetry systems, which are in contact with the patient skin. Bridge 3904 snaps onto the reusable pads 3902-3903 and provides a central mounting point for snapping on reusable wireless heart telemetry device 3905, which includes the hardware required to wirelessly transmit the heart telemetry data to the patient monitoring system 3906. This hardware includes, for example, but is not limited to, filtering, MCU, wireless SoC, battery, and isolation technology, as further described in FIG. 32. Further example embodiments permanently attach bridge 3904 to the wireless heart telemetry device 3905 as one re-useable device that snaps onto reusable pads 3902-3903. In the example embodiment shown in FIG. 39. the bridge, 3904, is a straight bridge.

One example embodiment of FIG. 39 utilizes BLUETOOTH (classic or low energy) as its wireless communication method, while other example embodiments use other wireless communications, such as, but not limited to, WI-FI, LORA, ZIGBEE, or other proprietary wireless communications methods. 3906, in the example embodiment shown in FIG. 39, is a bedside patient monitoring system. Other example embodiments use bedside systems connected to hospital wide patient monitoring systems, pocket patient monitoring systems connected to bedside and/or hospital wide patient monitoring systems, independent pocket patient monitoring systems, smart device patient monitoring systems, portable patient monitoring systems, or hospital wide patient monitoring systems, instead of bedside patient monitoring system 3906. The portion of the system worn on the user 3901 in FIG. 39 is designed to be waterproof for continuous patient monitoring, including damp locations, such as the bath or shower. Additionally, the example embodiment shown in FIG. 39 shows the example wireless ring pulse oximeter embodiment, 3907, worn on the subject's finger, 3908 to provide the patient monitoring system with the additional physiological information, including, but not limited to SpO₂, pleth graph, and heart rate (calculated from the pleth graph in addition to the ECG calculated heart rate).

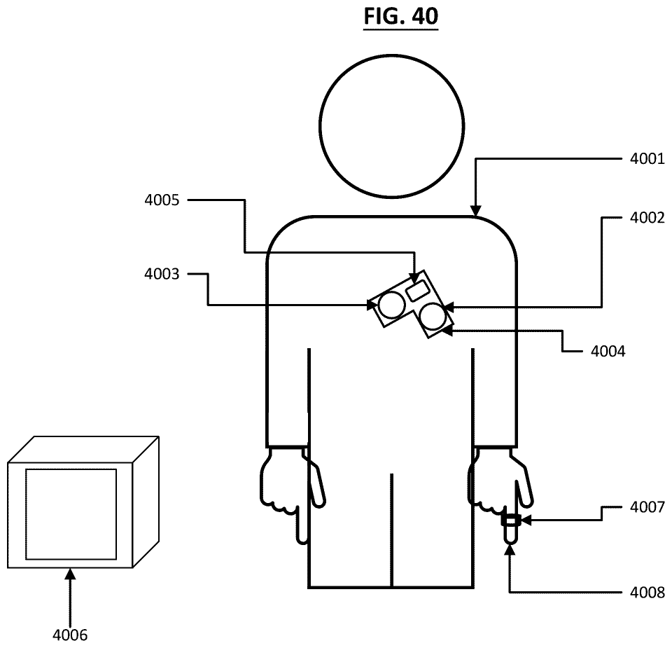

FIG. 40 shows an example embodiment of a wireless heart telemetry monitoring system with an example embodiment of wireless patient/subject worn conductive pads that include a central wireless heart telemetry electrode with an example right angle two pad clip, and includes an example ring embodiment for combined wireless pulse oximetry, as a patient monitoring system as further described in another section of this document. User 4001 wears heart monitor pads 4002-4003, which wirelessly sends data to patient monitoring system 4006, via a central wireless heart telemetry device 4005. The example locations of monitoring pads 4002-4003 are meant for demonstration purposes and are not to be considered the only locations or quantity of pads required for heart telemetry monitoring. As previously discussed, this example embodiment represents a single 2 electrode lead in the 3 electrode Einthoven's triangle monitoring arrangement. This arrangement produces the ECG graph for ECG lead 2, which is most commonly selected on hospital monitoring systems; however, additional pad locations are required for most advanced medical decisions and nurse monitoring stations.

One example embodiment adds the additional pads when necessary, utilizing wired or wireless methods, further described in other figures in this document. In the example shown, 4002-4003 utilize disposable snap on or clip on pads similar to those used in current heart telemetry systems, which are in contact with the patient skin. Bridge 4004 snaps onto the reusable pads 4002-4003 and provides a central mounting point for snapping on reusable wireless heart telemetry device 4005, which includes the hardware required to wirelessly transmit the heart telemetry data to the patient monitoring system 4006. This hardware includes for example, but is not limited to, filtering, MCU, wireless SoC, battery, and isolation technology, as further described in FIG. 32.

Further example embodiments permanently attach bridge 4004 to the wireless heart telemetry device 4005 as one re-useable device that snaps onto reusable pads 4002-4003. In the example embodiment shown in FIG. 40. the bridge, 4004, is a right-angle bridge to provide a better alignment of the pads relative to the heart in some patients. One example embodiment of FIG. 40 utilizes BLUETOOTH (classic or low energy) as its wireless communication method, while other example embodiments use other wireless communications, such as, but not limited to, WI-FI, LORA, ZIGBEE, or other proprietary wireless communications methods. 4006, in the example embodiment shown in FIG. 40, is a bedside patient monitoring system. Other example embodiments use bedside systems connected to hospital wide patient monitoring systems, pocket patient monitoring systems connected to bedside and/or hospital wide patient monitoring systems, independent pocket patient monitoring systems, smart device patient monitoring systems, portable patient monitoring systems, or hospital wide patient monitoring systems, instead of bedside patient monitoring system 4006.

The portion of the system worn on the user 4001 in FIG. 40 is designed to be waterproof for continuous patient monitoring, including damp locations, such as the bath or shower. Additionally, the example embodiment shown in FIG. 40 shows the example wireless ring pulse oximeter embodiment, 4007, worn on the subject's finger, 4008 to provide the patient monitoring system with the additional physiological information, including, but not limited, to SpO₂, pleth graph, and heart rate (calculated from the pleth graph in addition to the ECG calculated heart rate).

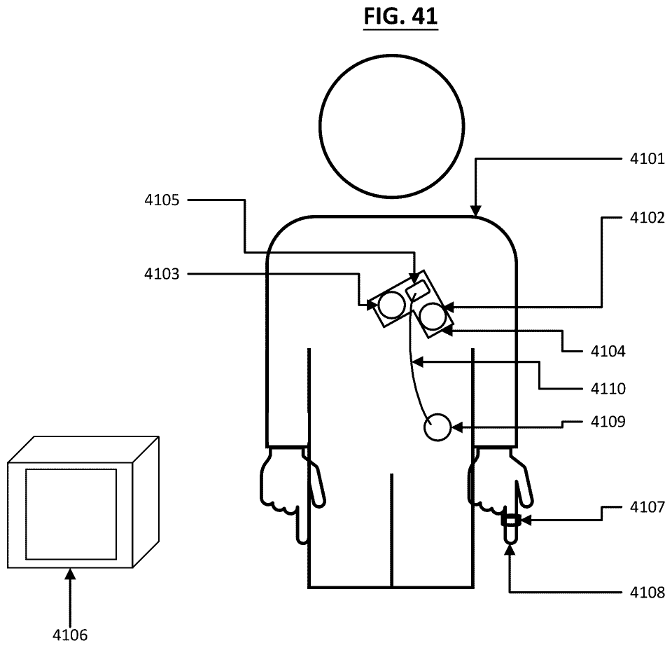

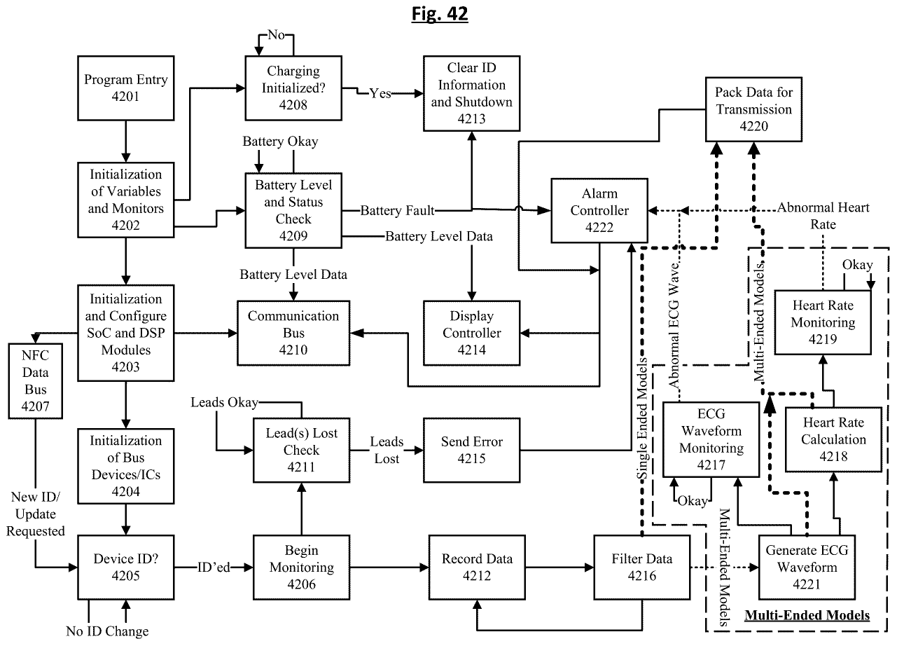

FIG. 41 shows an example embodiment of a wireless heart telemetry monitoring system with an example embodiment of wireless patient/subject worn conductive pads that include a central wireless heart telemetry electrode with an example right angle two pad clip with an additional under gown wired pad connected to the central wireless heart telemetry electrode, and includes an example ring embodiment for combined wireless pulse oximetry, as a patient monitoring system as further described in another section of this document. User 4101 wears heart monitor pads 4102-4103, which wirelessly sends data to patient monitoring system 4106, via a central wireless heart telemetry device 4105. The example locations of monitoring pads 4102-4103 are meant for demonstration purposes and are not to be considered the only locations or quantity of pads required for heart telemetry monitoring. As previously discussed, this example embodiment represents a single 2 electrode lead in the 3 electrode Einthoven's triangle monitoring arrangement.

This arrangement produces the ECG graph for ECG lead 2, which is most commonly selected on hospital monitoring systems; however, additional pad locations are required for most advanced medical decisions and nurse monitoring stations. The example embodiment shown adds an additional pad, 4109, when necessary, utilizing an under-gown wire, 4110, to connect it to the central wireless heart telemetry device 4105. Further example embodiments add additional pads when necessary, utilizing wired or wireless methods, further described in other figures in this document. Although this example embodiment, still utilizes cables to interconnect some of the reusable pads, it provides significant comfort and ease of use by including all cables under the subjects clothing or hospital gown, and prevents a large number of cables to be connected to a device worn externally to the patient. In the example shown, 4102-4103 and 4109 utilize disposable snap on or clip on pads, similar to those used in current heart telemetry systems, which are in contact with the patient skin.

Bridge 4104 snaps onto the reusable pads 4102-4103 and provides a central mounting point for snapping on reusable wireless heart telemetry device 4105, which includes the hardware required to wirelessly transmit the heart telemetry data to the patient monitoring system 4106. This hardware includes for example, but is not limited to, filtering, MCU, wireless SoC, battery, and isolation technology, as further described in FIG. 32. Further example embodiments permanently attach bridge 4104 to the wireless heart telemetry device 4105 as one re-useable device that snaps onto reusable pads 4102-4103. In the example embodiment shown in FIG. 41. the bridge, 4104, is a right-angle bridge to provide a better alignment of the pads relative to the heart in some patients.

One example embodiment of FIG. 41 utilizes BLUETOOTH (classic or low energy) as its wireless communication method, while other example embodiments use other wireless communications, such as, but not limited to, WI-FI, LORA, ZIGBEE, or other proprietary wireless communications methods. 4106, in the example embodiment shown in FIG. 41, is a bedside patient monitoring system. Other example embodiments use bedside systems connected to hospital wide patient monitoring systems, pocket patient monitoring systems connected to bedside and/or hospital wide patient monitoring systems, independent pocket patient monitoring systems, smart device patient monitoring systems, portable patient monitoring systems, or hospital wide patient monitoring systems, instead of bedside patient monitoring system 4106.

The portion of the system worn on the user 4101 in FIG. 41 is designed to be waterproof for continuous patient monitoring, including damp locations, such as the bath or shower. Additionally, the example embodiment shown in FIG. 41 shows the example wireless ring pulse oximeter embodiment, 4107, worn on the subject's finger, 4108 to provide the patient monitoring system with the additional physiological information, including, but not limited to SpO₂, pleth graph, and heart rate (calculated from the pleth graph in addition to the ECG calculated heart rate). It is important to note that the embodiments and the figures related to patient monitoring systems with ECG monitoring, as described in this document, may be used in part or in combination with each other to integrate multiple types of physiological information into a single system, as required based on the conditions being monitored.

### Additional Patient Monitoring System Embodiments

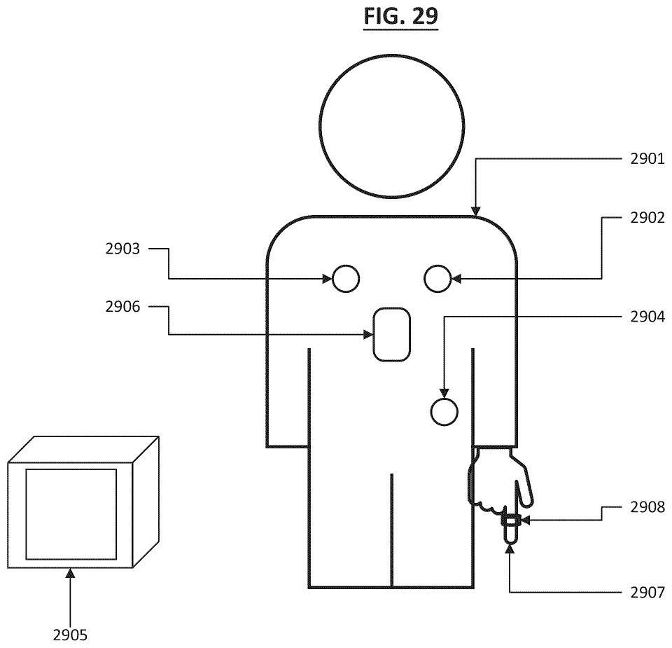

FIG. 29 shows an example embodiment of a wireless patient monitoring system including heart telemetry monitoring and melanin bias reducing pulse oximetry. User 2901 wears heart monitor pads 2902-2904, which wired or wirelessly sends data to wearable patient monitoring base point 2906. The example locations of monitoring pads 2902-2904 are meant for demonstration purposes and are not to be considered the only locations or quantity of pads required for heart telemetry monitoring. In the example shown, 2902-2904 utilize disposable snap on or clip on pads, which are in contact with the patient skin, similar to those used in current heart telemetry systems. Snapped or clipped onto these pads are the reusable devices, which include the hardware required to wired or wirelessly transmit the heart telemetry data to the wearable base point 2906. In wireless embodiments, this hardware includes, but is not limited to, filtering, MCU, wireless SoC, battery, and isolation technology.

The wireless signals from 2902-2904 must be synchronized, in order for the system to work properly using, for example, time of flight calculations and/or synchronized time stamping. One example embodiment of the wireless system, described in FIG. 29, utilizes independent grounds for 2902-2904, while other wireless example embodiments use an interconnected and centralized ground. In example wired embodiments, the signals from pads 2902-2904, including grounds, are connected directly to 2906, and 2906 includes the hardware described above in wireless embodiments. One example embodiment of wireless connectivity between 2902-2904 and 2906 utilizes BLUETOOTH as its communication method, while other example embodiments use other wireless communication methods, such as, but not limited to, LORA, NFC, RFID, or other proprietary wireless communication methods.

One example embodiment of 2906 utilizes BLUETOOTH as its wireless communication method to patient monitoring system 2905, while other example embodiments use other wireless communications, such as, but not limited to, WI-FI, LORA, or other proprietary wireless communication methods. In wireless hospital patient monitoring systems, 2905 uses similar communication methods, as described above, for wearable base point 2906. 2905, in the example embodiment shown in FIG. 29, is a wired or wireless bedside patient monitoring system. Other example embodiments use bedside systems connected to hospital wide patient monitoring systems, pocket patient monitoring systems connected to bedside and/or hospital wide patient monitoring systems, independent pocket patient monitoring systems, smart device patient monitoring systems, portable patient monitoring systems, or hospital wide patient monitoring systems, instead of bedside patient monitoring system 2905. 2905 utilizes wired or wireless methods of communication for all example embodiments listed above. Other example embodiments of 2805 use wired TCP/IP or other types of ethernet connections to connect to hospital patient monitoring systems, as described above. 2906, in the example embodiment shown, does not include a screen, in order to save battery life, while some example embodiments include a screen for convenience.

The example placement of 2906 is meant for demonstration purposes and is not to be considered the only location that is used. For example, patients that have pace makers or other implanted devices near or around the area shown, may not be able to have a wireless device in close proximity to their implant due to interference concerns. In situations where there are concerns for interference with an implant, 2902-2904 can be connected, via wired methods to 2906, and 2906 can be placed in a non-interfering location on the body. 2906, in the example embodiment shown in FIG. 29, is attached to user 2901, via medical grade adhesives, such as, but not limited to, temporary skin glue, tapes, and/or TEGADERM. In other example embodiments, 2906 is a pocket worn or belt clip worn device. 2907 represents the example appendage that the melanin bias reducing pulse oximetry portion 2908 is placed on.

As described above, 2907 is a finger (shown in FIG. 29), wrist, ankle, arm, leg, or other appendage, as described previously in the finger clip, ring (shown in FIG. 29), and bracelet embodiments, of the pulse oximeter 2908. 2908, as shown as the ring embodiment in FIG. 29 wired or wirelessly communicates with wearable base point 2906 or directly with patient monitoring system 2905, depending on the requirements of the embodiment. In wireless embodiments, 2908 communicates using the wireless communication methods, described in FIG. 29, and elsewhere in this document discussing melanin bias reducing pulse oximetry embodiments to provide a continuous 5^{th} vital sign in monitoring applications. The portion of the system worn on the user 2901 in FIG. 29 is designed to be waterproof for continuous patient monitoring, including damp locations, such as the bath or shower. Further, other example embodiments are also intrinsically sealed for continuous monitoring in hazardous situations such as, for example, in-patient settings and also in the field on emergency personnel as a real time vitals monitoring system.

Other example embodiments use for example, but not limited to, cellular or emergency digital radio interfaces to transmit emergency personnel and military vital information using the heart telemetry and melanin bias reducing system described herein. Further, other example embodiments have reflective melanin bias reducing pulse oximetry systems, as described previously in this document, built into the wearable base point 2906 in example embodiments where the base point is attached directly to the skin.

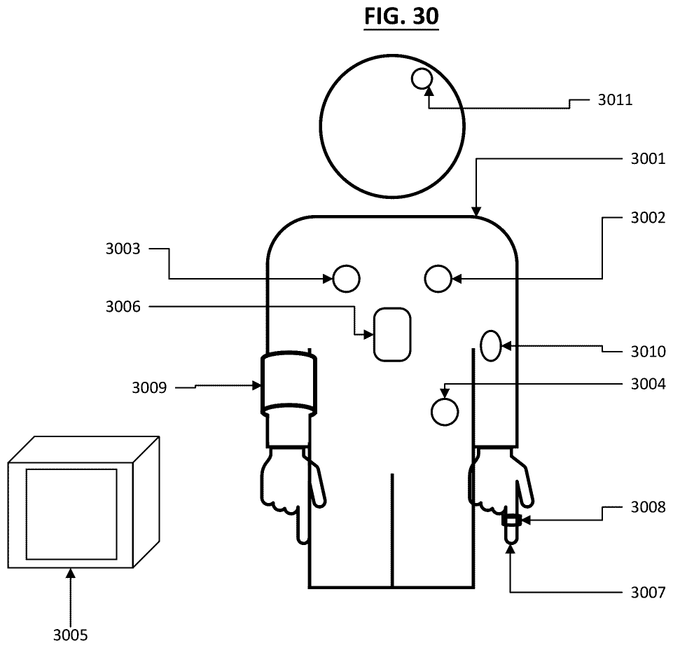

FIG. 30 shows another example embodiment of a wireless patient monitoring system including heart telemetry monitoring, pulse oximetry or melanin bias reducing pulse oximetry, blood pressure monitoring, temperature monitoring, and glucose (blood sugar) monitoring. User 3001 wears heart monitor pads 3002-3004, which wired or wirelessly sends data to wearable patient monitoring base point 3006. The example locations of monitoring pads 3002-3004 are meant for demonstration purposes and are not to be considered the only locations or quantity of pads required for heart telemetry monitoring. In the example shown, 3002-3004 utilize disposable snap on or clip on pads, which are in contact with the patient skin, similar to those used in current heart telemetry systems. Snapped or clipped onto these pads are the reusable devices, which include the hardware required to wired or wirelessly transmit the heart telemetry data to the wearable base point 3006.

In wireless embodiments, this hardware includes, but is not limited to, filtering, one or more MCUs (which includes ADC [analog to digital conversion] hardware internally or externally to the MCU), wireless SoC, battery, and isolation technology. The wireless signals from 3002-3004 must be synchronized, in order for the system to work properly using, for example, time of flight calculations and/or synchronized time stamping. One example embodiment of the wireless system, described in FIG. 30, utilizes independent grounds for 3002-3004, while other wireless example embodiments use an interconnected and centralized ground. In example wired embodiments, the signals from pads 3002-3004, including grounds, are connected directly to 3006, and 3006 includes the hardware described in wireless heart telemetry article and wireless pulse oximeter article embodiments. One example embodiment of wireless connectivity between 3002-3004 and 3006 utilizes BLUETOOTH as its communication method, while other example embodiments use other wireless communication methods, such as, but not limited to, LORA, NFC, RFID, or other proprietary wireless communication methods. One example embodiment of 3006 utilizes BLUETOOTH as its wireless communication method to patient monitoring system 3005, while other example embodiments use other wireless communications, such as, but not limited to, WI-FI, LORA, or other proprietary wireless communication methods.

In wireless hospital patient monitoring systems, 3005 uses similar communication methods, as described above, for wearable base point 3006. 3005, in the example embodiment shown in FIG. 30, is a wired or wireless bedside patient monitoring system. Other example embodiments use bedside systems connected to hospital wide patient monitoring systems, pocket patient monitoring systems connected to bedside and/or hospital wide patient monitoring systems, independent pocket patient monitoring systems, smart device patient monitoring systems, portable patient monitoring systems, or hospital wide patient monitoring systems, instead of using bedside patient monitoring system 3005. 3005 utilizes wired or wireless methods of communication for all example embodiments listed above.

Other example embodiments of 3005 use wired TCP/IP or other types of ethernet connections to connect to hospital patient monitoring systems, as described above. 3006, in the example embodiment shown, does not include a screen, in order to save battery life, while some example embodiments include a screen for convenience. The example placement of 3006 is meant for demonstration purposes and is not to be considered the only location that is used. For example, patients that have pacemakers or other implanted devices near or around the area shown, may not be able to have a wireless device in close proximity to their implant due to interference concerns. In situations where there are concerns for interference with an implant, 3002-3004 can be connected, via wired methods to 3006, and 3006 can be placed in a non-interfering location on the body. 3006, in the example embodiment shown in FIG. 30, is attached to user 3001, via medical grade adhesives, such as, but not limited to, temporary skin glue, tapes, and/or TEGADERM. In other example embodiments, 3006 is a pocket worn or belt clip worn device. 3007 represents the example appendage that the pulse oximeter or the melanin bias reducing pulse oximetry portion 3008 is placed on.

As described above, 3007 is a finger (shown in FIG. 30), wrist, ankle, arm, leg, or other appendage, as described previously in the finger clip, ring (shown in FIG. 30), and bracelet embodiments of the pulse oximeter 3008. 3008, as shown as the ring embodiment in FIG. 30 wired or wirelessly communicates with wearable base point 3006 or directly with patient monitoring system 3005, depending on the requirements of the embodiment. In wireless embodiments, 3008 communicates using the wireless communication methods, described in FIG. 30, and elsewhere in this document discussing melanin bias reducing pulse oximetry embodiments to provide a continuous 5^{th} vital sign in monitoring applications.

The portion of the system worn on the user 3001 in FIG. 30 is designed to be waterproof for continuous patient monitoring, including damp locations, such as the bath or shower. Further, other example embodiments are also intrinsically sealed for continuous monitoring in hazardous situations such as, for example, in-patient settings and also hazardous conditions in the field on emergency personnel as a real time vitals monitoring system. Other example embodiments use for example, but not limited to, cellular or emergency digital radio interfaces to transmit emergency personnel and military vital information using the heart telemetry and melanin bias reducing system described herein. Further, other example embodiments have reflective melanin bias reducing pulse oximetry systems, as described previously in this document, built into the wearable base point 3006 in example embodiments where the base point is attached directly to the skin. 3009 is a blood pressure (BP) cuff used to determine blood pressure (BP) as previously discussed. In the example embodiment BP cuff, 3009, uses a wireless system to transmit information to both 3006 and 3005. 3009, includes the required hardware for wirelessly measuring BP including, but not limited to, one or more MCUs (which includes ADC [analog to digital conversion] hardware internally or externally to the MCU), pump, air valve, BP cuff, pressure sensor(s), battery, filtering, and wireless communication systems such as, but not limited to BLUETOOTH.

The location of 3009 in FIG. 30 is to be considered an example location, as there are many other locations a BP cuff can be placed. 3010 is a real-time glucose monitor patch used to determine blood sugar (glucose) as previously discussed. In the example embodiment real-time glucose monitor, 3010, uses a wireless system to transmit information to both 3006 and 3005. 3010, includes the required hardware for wirelessly measuring blood glucose levels (blood sugar levels) including, but not limited to, one or more MCUs (which includes ADC [analog to digital conversion] hardware internally or externally to the MCU), glucose monitor, battery, filtering, and wireless communication systems such as, but not limited to BLUETOOTH. 3010 is similar to the glucose monitors on the market, but with a communication system designed to work with the communication systems discussed in this document for patient monitoring. In other example embodiments, 3010, is not necessary as the pulse oximeter 3008 also includes non-invasive optical glucose sensing technology.

The location of 3010 in FIG. 30 is to be considered an example location, as there are many other locations a continuous glucose monitor can be placed. 3011 is a temperature sensor used to determine temperature as previously discussed. In the example embodiment temperature sensor, 3011, uses a wireless system to transmit information to both 3006 and 3005. 3011, includes the required hardware for wirelessly measuring temperature including, but not limited to, one or more MCUs (which includes ADC [analog to digital conversion] hardware internally or externally to the MCU), temperature sensor(s), battery, filtering, and wireless communication systems such as, but not limited to BLUETOOTH. The location of 3011 in FIG. 30 is to be considered an example location, as there are other locations a temperature sensor can be placed other than a temporal artery sensor as shown. These systems can also act as a motion and location tracking system when additional hardware is added to 3008 as previously discussed in this document.

Each device in the wireless network is assigned an ID for determining which patient it is monitoring to ensure the correct information is displayed for each patient in the monitoring network. In an example embodiment, these IDs are assigned using the UI (user interface) of each device. In another example embodiment, these IDs are assigned using an RFID or NFC assignment device that communicates with each device as it is connected to the patient. In another example embodiment, these IDs are assigned to devices in a particular room or bay and are configured during initial system set up.

### Heart Telemetry Article

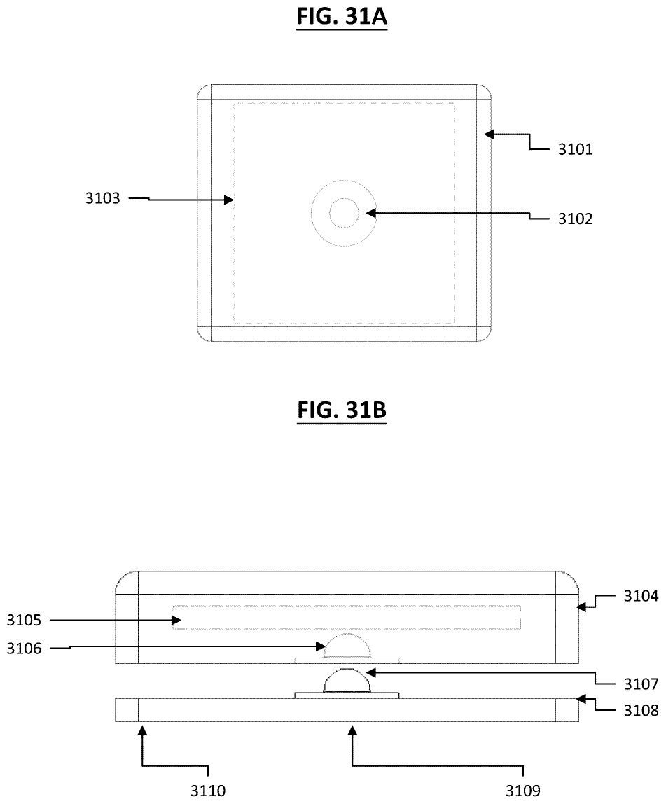

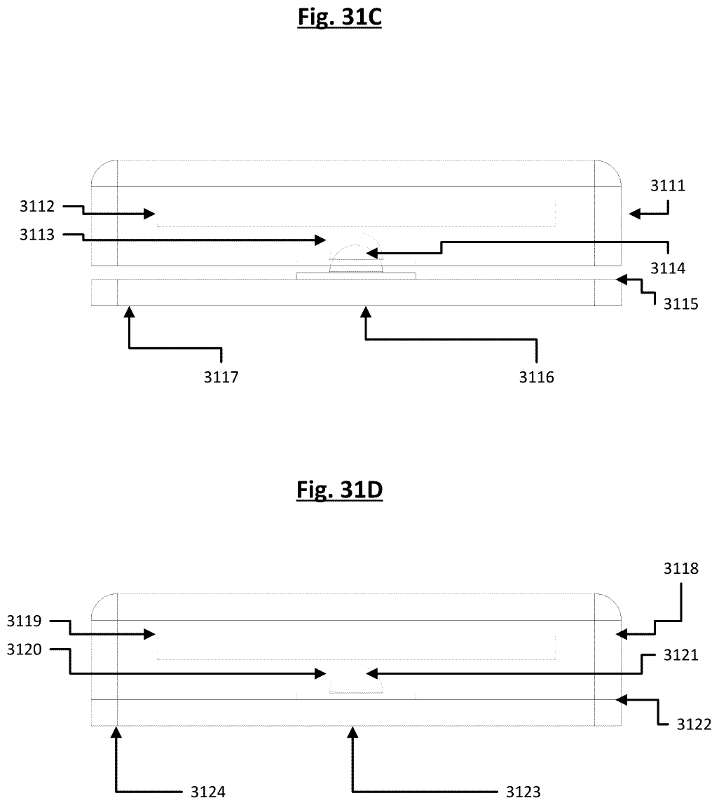

FIG. 31A-31D shows top and side view diagrams of an example embodiment of a wireless heart telemetry pad or electrode, as well as the mating of the disposable and reusable portions of the wireless heart telemetry electrode, in accordance with the invention. FIG. 31A shows a top view of the example reuseable heart telemetry electrode, which includes the connections to the single use heart telemetry pad (shown in 31B as 3108). FIG. 31B shows a side view of the example reuseable heart telemetry electrode and the side view of the single use heart telemetry pad, which is similar to the pads in use with current heart telemetry (ECG) systems. FIG. 31A also exposes the internal electronics compartment and the electrical connection to the pad. 3101 is the case of the reusable wireless heart telemetry electrode. The case, 3101, is produced in a fashion that waterproofs the electronics and creates an easy to clean design. Case, 3101, also includes magnetic charging ports (not shown in FIG. 31A), waterproof USB ports (not shown in FIG. 31A), or utilizes a wireless charging method. 3101, also includes a user interface for pairing and ID(ing) of the telemetry pads. In an example embodiment, the wireless pads are sold in a networked set with specific electrodes designed for using in predetermined locations based on the heart activity they are required to detect to properly produce an ECG graph. In an example embodiment, the overall networked set, is assigned an ID to relate it to the patient it is monitoring.

In another example embodiment, each wireless telemetry electrode device is assigned an ID to relate it to the patient it is monitoring and it is assigned a location on the patient's body that it is placed. In an example embodiment, these IDs and/or locations are assigned using the UI (user interface) of each device. In another example embodiment, these IDs and/or locations are assigned using an RFID or NFC assignment device that communicates with each device as it is connected to the patient. In another example embodiment, these IDs and/or locations are assigned to devices in a particular room or bay and are configured during initial system set up. 3102 is an example embodiment of the electrode connection point, which snaps onto the pad connection “button”, 3107.

Other example embodiments use other connection methods, such as, but not limited to, clips, buttons, and cabled leads. 3103, represents the electronics compartment in the telemetry device which is further described in FIG. 32 and also was previously described in other sections of this document. 3104 is the case of the reusable wireless heart telemetry electrode and is the same as 3101 as previously discussed. 3105, represents the electronics compartment in the telemetry device, which is further described in FIG. 32, and was also previously described in other sections of this document, and is the same as 3103 as previously discussed. 3106 is an example embodiment of the electrode connection point, which snaps onto the pad connection “button”, 3107 and 3106 are the same as 3102 as previously discussed. 3108 represents the single use telemetry pad as previously discussed and is similar to the pads in use on current heart telemetry systems. Pad 3108 as shown is to be an example of the type, connection method, and style of heart telemetry pads used. 3107 is an example of the pad connection “button”, that connects with the example embodiment of the electrode connection point 3106 (also 3102).

Other example embodiments use other types of pads which utilize other connection methods, such as, but not limited to, clips, buttons, and cabled leads. 3109 represents the conductive electrode that connects the telemetry device to the patient. 3109 is designed and functions similar to the patient connection point on current reusable telemetry pads. 3109 is in direct contact with the patient's skin. 3110, represents the area of the pad around 3109, which is covered in an adhesive material to ensure the pad stays attached to the patient and ensures adequate connection to the patient at 3109. FIG. 31C shows the same view and components as FIG. 31B, however, it shows the example reuseable heart telemetry electrode and the side view of the single use heart telemetry pad in their partially mated position.

The partially mated position shows the “button”, 3114, on the disposable pad beginning the connection with the example embodiment of the electrode connection point 3113. 3111 is the case of the reusable wireless heart telemetry electrode and is the same as 3101 as previously discussed. 3112, represents the electronics compartment in the telemetry device, which is further described in FIG. 32, and was also previously described in other sections of this document, and is the same as 3103 as previously discussed. 3113 is an example embodiment of the electrode connection point, which snaps onto the pad connection “button”, 3114 and 3113 are the same as 3102, as previously discussed.

3115 represents the single use telemetry pad as previously discussed and is similar to the pads in use on current heart telemetry systems. Pad 3115, as shown, is to be an example of the type, connection method, and style of heart telemetry pads used. 3114 is an example of the pad connection “button”, that connects with the example embodiment of the electrode connection point 3113 (also 3102). Other example embodiments use other types of pads which utilize other connection methods, such as, but not limited to, clips, buttons, and cabled leads. 3116 represents the conductive electrode that connects the telemetry device to the patient. 3116 is designed and functions similar to the patient connection point on current reusable telemetry pads. 3116 is in direct contact with the patient's skin. 3117, represents the area of the pad around 3116, which is covered in an adhesive material to ensure the pad stays attached to the patient and ensures adequate connection to the patient at 3116. FIG. 31D shows the same view and components as FIG. 31B, however, it shows the example reuseable heart telemetry electrode and the side view of the single use heart telemetry pad in their fully mated position as the “button”, 3121, on the disposable pad connects with the example embodiment of the electrode connection point 3120.

3118 is the case of the reusable wireless heart telemetry electrode and is the same as 3101 as previously discussed. 3119, represents the electronics compartment in the telemetry device, which is further described in FIG. 32, and was also previously described in other sections of this document, and is the same as 3103 as previously discussed. 3120 is an example embodiment of the electrode connection point, which snaps onto the pad connection “button”, 3121 and 3120 are the same as 3102, as previously discussed. 3122 represents the single use telemetry pad, as previously discussed, and is similar to the pads in use on current heart telemetry systems. Pad 3122 as shown is to be an example of the type, connection method, and style of heart telemetry pads used. 3121 is an example of the pad connection “button” that connects with the example embodiment of the electrode connection point 3120 (also 3102).

Other example embodiments use other types of pads, which utilize other connection methods, such as, but not limited to, clips, buttons, and cabled leads. 3123 represents the conductive electrode that connects the telemetry device to the patient. 3123 is designed and functions similar to the patient connection point on current reusable telemetry pads. 3123 is in direct contact with the patient's skin. 3124, represents the area of the pad around 3123, which is covered in an adhesive material to ensure the pad stays attached to the patient and ensures adequate connection to the patient at 3123.

FIG. 32 shows a diagram of an overview of an example general hardware signal flow of an example wireless heart telemetry pad or electrode. Hardware in FIG. 32 is represented in previous figures as 3103 and 3105 and previously discussed in other figures in this document when discussing patient monitoring systems and heart telemetry. In an example embodiment user, 3212, interacts with the UI (user interface) displayed on LCD screen, 3210, via user button, 3209. In other example embodiments no LCD screen, 3210, is included to conserve battery life. In these style embodiments user button, 3209, includes different lengths and styles of presses to access different functions. Further, in other example embodiments, no user button, 3209, is included and NFC/RFID or other wireless means of configuring the device are used, as further described in this section.

User, 3212, also interacts with the heart telemetry device, via NFC/RFID communication link, 3211. NFC/RFID is used to program and modify settings of the heart telemetry device using an external programmer or smart device with appropriate software and hardware. In another example embodiment, 3211 is used to assign a device ID as previously described. The heart telemetry device is powered, via a Lipo battery, 3218, in one example embodiment. In another example embodiment, other power sources such as, but not limited, to coin cell batteries or other rechargeable battery chemistries are used. Lipo battery 3218 is used in the example embodiment due to its efficiency and energy density. Other example embodiments use other types of batteries, such as, alkaline, nickel cadmium, lead acid, AGM, gel, lithium ion, lithium phosphate, solid state, sodium composition, ceramic, kinetic, and/or removable rechargeable batteries. Further example embodiments use a removable battery, 3218, so the battery can be easily changed on a patient in long-term monitoring situations. In these example embodiments, a battery charging station is used to charge the battery and much of the onboard battery charging and power management logic is not included.

Battery, 3218, is monitored, via temperature sensor, 3217, and charging logic and management, 3215. 3215, in one example embodiment are purpose-built ICs. Other example embodiments use other forms for charging logic and management, such as, but not limited to, MCU driven charging, constant current charging, USB diode-controlled charging, and/or removable batteries with external chargers. Battery meter logic 3213 reports battery usage information to MCU 3206. In the example embodiment shown, 3213 is based off a voltage divider design, however, other example embodiments use other battery meter methods, such as, but not limited to, state of charge ICs, fuel gauge ICs, and/or Coulomb counters.

Programming system 3216 communicates with the MCU 3206 to perform software updates. In other example embodiments, programming system 3216 uses a specialized programmer bootloader IC (not shown), in order to update firmware on MCU 3206, using data from the USB port (not shown) or via over-air-updates. Some example commercial embodiments, which do not require the user to have direct firmware access, do not include the programming system, 3216, and its peripherals or independent power management logic (part of 3214). Independent latching circuitry in power management logic 3214 is required because microcontroller (MCU), 3206, has a delayed boot while waiting for programming parts of the programming system 3216 to release it from program waiting mode (as discussed previously in reference to the pulse oximeter figures). Other example embodiments of the hardware use other programming and firmware update methods than those shown, such as, but not limited to, ICSP, JTAG, SWD, UART, SPI, parallel, and/or wireless update methods, some of which do not require dedicated programmer bootloader ICs or independent power latching circuitry, as described above.

MCU 3206 in the example embodiment shown is an NXP Kinetis ARM Cortex M4 running the Teensy Arduino platform with a clock speed of 72 MHz generated by scaling 16 MHz program clock 3207. The Kinetis MCU 3206 is chosen for the example embodiment shown due to the on-board 12-bit ADC (shown abstracted separately outside the MCU, 3206, in FIG. 32), so that the circuit would require minimal external components. Other example embodiments use other MCU ICs instead of the Kinetis MCU 3206 (also discussed in 2207), such as, but not limited to, Nordic ICs, other Arduino compatible ICs, and other microcontrollers with suitable peripherals known to those in the field. MCU 3206 runs the software required for the wireless heart telemetry pads, which controls the hardware peripherals and inputs and outputs.

Power Regulation 3219 provides appropriate voltages to the electronic components in the hardware, as well as provides power filtering required to ensure high frequency digital signals do not interfere with the analog signal data collection required in heart telemetry. Wireless transceiver 3208 represents the BLUETOOTH Low Energy (BLE) transceiver for transmitting the heart telemetry data and information to the base station, receiver, or patient monitoring system, as previously described. In other embodiments, wireless transceiver, 3208, represents other wireless communications, such as, but not limited to, BLUETOOTH mesh, THREAD, ZIGBEE, 802.15.4, ANT, 2.4 GHz proprietary SoC stacks, BLUETOOTH classic, WI-FI, LORA, or other proprietary wireless communication methods. In the embodiment shown, wireless transceiver, 3208, is separated from the MCU, 3206, and represents external ICs or a co-processor responsible for the wireless communication in order to remove the wireless overhead from MCU, 3206.

In further embodiments, wireless transceiver, 3208, is included in the MCU, 3206, to save valuable PCB space, reduce power consumption, and reduce component counts. In the embodiment shown, ADC (analog to digital converter) 3205, is abstracted out of the MCU separately for illustration purposes. However, as previously mentioned in this document, ADC 3205 is built into MCU, 3206, for the purpose of the embodiment being discussed. In other example embodiments, that use MCUs that do not have built-in ADCs or require a high-resolution standalone ADC IC, ADC 3205, is external to MCU, 3206. Filtering 3204 represents the signal filtering performed in hardware on the heart telemetry signal. Filtering 3204, includes for example, analog filters including, but not limited to, high pass, low pass, notch, low cut, and high cut filters.

Additionally, example embodiments include common mode rejection as part of the filtering process. Further example embodiments perform signal filtering in software or firmware on MCU, 3206, in lieu of 3204 or in conjunction with 3204, as further described in FIG. 42. Isolation 3203, provides electrical isolation between the device and the patient. Additional example embodiments do not include this type of additional hardware isolation. Amplification 3202 represents the hardware used to amplify the small heart electrical signals from the patient, 3220, received from the pad input, 3201. An example embodiment utilizes, for example, op amps, instrumentation amplifiers, and/or differential amplifiers for amplification stage, 3202. Pad input, 3201, represents the electrical input from 3102 (also 3106) connected to 3107 and 3109. In other example embodiments, the control signal passing from amplification, 3202, to MCU, 3206, is also electrically isolated for increased patient protection. Further, in other example embodiments, 3202, 3203, and 3204 are all included on one digital signal processing or standalone ECG IC.

One example embodiment utilizes an AD8232 for a standalone ECG IC. In other example embodiments, ADC, 3205, is also included in one DSP IC along with 3202, 3203, and 3204 rather than being a separate IC or being included in MCU, 3206. Heart electrical signals from the patient, 3220, received by pad input, 3201, are collected by current heart telemetry systems and displayed as lead signals, as previously described, by using differential signals and software reference signals. In the fully wireless example embodiment described, these signals are collected as single ended signals and are processed by MCU, 3206, or the processor at the receiver or patient monitoring system, in order to be represented graphically in the same way current ECG systems display signals by recombining signals required to be differential in software.

Depending on the implementation required for the embodiment, the signals collected at each heart telemetry pad will be transmitted as raw data with minimal processing or hardware signal filtering performed locally to reduce the component count, power consumption, and the processing overhead, or these signals may be processed before transmission. Not shown in the figure is the method of charging battery 3218. Example embodiments utilize many charging methods, for example but not limited to, magnetic charging, inductive charging, micro-USB, mini-USB, USB-C, waterproof micro-USB, waterproof mini-USB, waterproof USB-C, QI wireless charging, and charging contacts on a charging cradle. Additionally, hospital grade devices require shielding both against outside interference and to prevent unwanted interference with other medical devices or implants. Example embodiments utilize many methods of shielding, including, but not limited to, MRI hardening, electromagnetic interference shielding, and/or radio frequency interference shielding. Examples of this shielding include a grounded shield placed over the electronic components, conductive sprayed coating inside the case of the device, and/or braided shielding around openings in the device case.

FIG. 42 shows a general software flow, in accordance with the invention, of an example wireless heart telemetry device. The software flow diagram shows differences between single ended embodiments, for example, FIGS. 27, 28, 29, and 30, versus multi-ended embodiments, for example, FIGS. 38, 39, 40, and 41. Single ended embodiments include, for example, wireless heart telemetry patches which only receive signals from a single patient heart telemetry pad. In these single ended embodiments, the filtered data, 4216, is immediately packed for transmission and sent to the patient monitoring system. In these embodiments, the patient monitoring system recombines the single ended information to create the ECG “leads” previously described to generate the ECG waveforms and heart rate information. In some example embodiments the patient respiration rate is also generated from the ECG information. Multi-ended embodiments include, for example, wireless heart telemetry patches which receive signals from multiple patient heart telemetry pads. In these multi-ended embodiments, the filtered data, 4216, is used for the ECG “leads” previously described to generate the ECG waveforms and heart rate information prior to wireless data transmission.

The software starts at program entry point 4201, which in the example embodiment is a predefined boot address or in another example embodiment is transferred from the MCU bootloader. In some example embodiments, a bootloader is used to listen for wireless software updates prior to the main program starting. The software initializes all variables and monitoring loops in block 4202. The monitoring loops, for example, are run concurrently with the main program for real-time monitoring operations. Example embodiments contain monitors which include, for example, charging monitor 4208, battery level and status monitoring 4209, and system watch dog monitoring (not shown). The program initializes and configures the System-On-Chip (SoC) and digital signal processing (DSP) modules in block 4203, which in some example embodiments include, for example, near field communication (NFC), communications bus, and DSP filtering engines (not shown). Example embodiments of the communications module are described in the wireless hardware sections of this document.

The software initializes external devices or ICs on hardware communication buses that require configuration or initialization at startup in block 4204, as further described in the hardware sections of this document. In the example embodiment described, charging monitor 4208, runs in a loop waiting for the device to be connected to or placed on a charger. If no charger is detected, the loop will run until the device's battery runs low, or the device is turned off (not shown). If a charger is detected, the charging monitor, 4208, moves to block 4213 which deletes the device ID information and shuts the device down for charging mode. In the example embodiment shown, the ID information is cleared, since the example device can't be used while charging and may be used on a different patient after charging. The ID deletion also prevents an ID conflict when a device with a fresh battery is assigned to continue monitoring the patient. In some example embodiments, the ID is transferred to the new device that will continue monitoring the patient, via NFC bus, 4207, on both heart telemetry devices, or via the heart telemetry ID assignment and configuration device (not shown).

Battery monitor block, 4209, in the example embodiment monitors the battery level, which is transmitted to the patient monitoring system, via, for example, communications bus 4210. In some example embodiments, the battery level is displayed on the heart telemetry device, via, for example, display controller 4214, or via a momentary user enabled battery meter bar (not shown). Battery monitor block, 4209, in the example embodiment monitors for battery faults, such as, for example, over temperature or critically low battery levels. In the example embodiment, a battery fault triggers an alarm, via alarm controller 4222 and also triggers a shutdown, via block 4213. Communications bus 4120, in the example embodiment, is the data interface to the wireless SoC or the hardware wireless controller, which handles the transmission and receival of data. Near Field Communication (NFC) data bus 4207, in the example embodiment is used to receive ID information and for general device configuration (not shown). Some example embodiments received the configuration information and ID from, for example, a smart device with a configuration application, a dedicated configuration device, a nurse station computer linked to the patient's charting information, the patient monitor at the bedside or clipped to the patient, or another already configured wireless heart telemetry device. Block 4205, in the example embodiment shown, starts a loop waiting for an ID assignment after initial bootup is complete.

If an ID was already assigned due to, for example, a reboot, or if a new ID was configured, via, for example, NFC data bus, 4207, the software enters its configured state and is ready to begin patient monitoring operations in block 4206. Other example embodiments utilize RFID, WI-FI, a centralized patient monitoring configuration utility, or other proprietary configuration method instead of NFC to configure the device information and ID.

In the example embodiment, the lead(s) lost check block, 4211, runs concurrently in a loop with the main program when monitoring begins (4206). The leads lost loop runs to ensure the electrodes are making proper contact with the patient and produces an error, 4215, to trigger a warning alarm, 4222, when the electrodes are no longer in contact with the patient causing a lead lost condition. In the example embodiment, after monitoring begins, the patient's ECG heart signal data is recorded in block 4212 and filtered in 4216, as described in the hardware section for how the analog filtering methods are implemented in software. In single ended embodiments, the filtered data is sent to block 4220 to be packed into the communication protocol for transmission by communication block 4210 or displayed, via display controller 4214 in example embodiments that include an on-board screen. A loop to monitor the patient is started which loops, for example between recording data, 4212, and filtering data 4216 to be transmitted.

Many embodiments described in this document do not have a built-in screen or display controller, 4214, as described in the hardware sections to save power and increase battery life. In such example embodiments, the data is only transmitted wirelessly and is displayed on the patient monitoring system. In multi-ended embodiments, the filtered data 4216, is sent in a constant stream to the ECG waveform generator block, 4221, which uses the data from each connected electrode to generate the ECG leads previously described and to create the ECG graph for each lead required.

Additionally, in the example multi-ended embodiment shown, the waveform generator block, 4221, sends the waveforms to block 4220 to be packed for transmission by communication bus block 4210 or displayed, via display controller 4214, in example embodiments with a built-in screen. In the example embodiment shown the generated ECG waveform, 4221, is sent to concurrently running heart rate calculation block 4218 and ECG waveform monitoring block 4217. In the example embodiment shown, the heart rate data is calculated based on the QRS complex waveform R peaks in block 4218 and monitored concurrently for abnormal heart rates in heart rate monitoring block 4219. In the example embodiment, if an abnormal heart rate is detected alarm controller 4222 is notified. In other example embodiments, respiration rate calculation and monitoring is also performed by blocks 4218 and 4219 respectively. In the example embodiment, ECG waveforms are monitored concurrently in block 4217, and abnormal waveforms send a notification to alarm controller 4222.

Alarm controller 4222, filters alarms based on user requirements configured at device boot up and sends any alarms to communication bus 4210 to be reported visually at the patient monitoring system. In some additional embodiments, alarms are reported audibly by use of various tones at the patient monitoring system. In some example embodiments the alarm controller reports the alerts to the built-in display controller, 4214, or audibly to the built-in alarm (not shown). As shown in the figure, blocks 4221, 4217, 4218, and 4219 are only used in embodiments that utilize multi-ended hardware as previously described.

### OTHER EXAMPLE EMBODIMENTS

Other example embodiments use portions of the embodiments disclosed in this document to create a comfortable and easy to use child monitoring system, which gives parents access to real time location, including geo-fencing, melanin bias reducing pulse oximetry, and heart rate data. Further, other example embodiments use portions of the embodiments disclosed in this document to create a comfortable, fashionable, and easy to use way to incorporate melanin bias reducing pulse oximetry and heart rate monitoring into smart phone systems. Other example embodiments use portions of the embodiments disclosed in this document to create an alert system to replace or augment current fall detection, bed alarm, and call systems used in homes, nursing home facilities, hospitals, and other medical facilities. Other example embodiments are classified as IoT devices and connect to the internet through standalone communications, such as WI-FI or cellular communication methods or through smart devices to transmit patient monitoring information to physicians or monitoring services to allow the patient to be monitored globally without in-person visits.

Other example embodiments include the melanin bias reducing pulse oximeter technology discussed in this document in smart devices, such as for example smart watches, exercise equipment FITBITs, Holter monitors, smart rings, smart bracelets, and/or other health and fitness devices. Further, other example embodiments include emergency personnel (EMS, police, fire, etc.) and military melanin bias reducing pulse oximetry and heart telemetry monitor systems which interface over cellular or digital emergency service and/or military digital radio communications to provide continuous monitoring including a 5^{th} vital sign in the form of blood oxygen saturation levels. Further other embodiments provide a method for monitoring patients utilizing prescription pain killers such as opioids to provide audible and remote alarms when oxygen saturation levels reach a critical level, which may indicate a reaction or overdose. These embodiments also include connectivity to remotely alert an emergency contact or EMS.

The invention is described in terms of example hardware and software embodiments. The summarized and detailed descriptions of both the hardware and the software are not intended to limit the scope of the invention. The invention is used as a whole or in part for many other types of consumer and industrial devices as well.

While various embodiments of the invention have been described, the description is intended to be exemplary, rather than limiting, and it will be apparent to those of ordinary skill in the art that many more embodiments and implementations are possible that are within the scope of the invention. Accordingly, the invention is not to be restricted except in light of the attached claims and their equivalents based on the information provided in this patent application. Also, various modifications, combinations, and changes may be made within the scope of the attached claims.

1. A patient monitoring system for reporting and determining physiological information, the system comprising:
a plurality of wireless heart telemetry electrodes configured to detect electrical heart activity signals;
an analog to digital converter (ADC) configured to convert the electrical heart activity signals to digital information;
a communication interface configured to communicate digital information associated with the electrical heart activity signals as a type of physiological information over a communication network; and
one or more processors in communication with the analog to digital converter (ADC) and the communication interface, the one or more processors configured to:
receive, from the ADC via the communication interface, the digital information associated with the electrical heart activity signals, and
filter ECG (EKG or electrocardiogram) graphs based on the electrical heart activity signals received from the ADC.

2. A wireless heart telemetry electrode configured to detect the electrical heart signals representing electrical heart activity, the wireless heart telemetry electrode comprising:
a pad including a button configured to couple with an electrode connection point;
a analog to digital converter (ADC) configured to convert the electrical heart activity signals to digital information;
a communication interface configured to communicate digital information associated with the electrical heart activity signals as a type of physiological information over a communication network; and
one or more processors in communication with the ADC and the communication interface,
wherein ECG (EKG or electrocardiogram) graphs are filtered and determined by the one or more processors based on the electrical heart activity signals received from the at least one ADC.
